(19) United States
(12) Patent Application Publication
(10) Pub. No.: US 2022/0046923 A1
(43) Pub. Date: Feb. 17, 2022

(54) HERBICIDAL CINNOLINIUM COMPOUNDS

(71) Applicant: SYNGENTA CROP PROTECTION AG, Basel (CH)

(72) Inventors: James Nicholas SCUTT, Bracknell, Berkshire (GB); Nigel James WILLETTS, Bracknell, Berkshire (GB); Sean NG, Bracknell, Berkshire (GB)

(73) Assignee: SYNGENTA CROP PROTECTION AG, Basel (CH)

(21) Appl. No.: 17/414,125

(22) PCT Filed: Dec. 17, 2019
(86) PCT No.: PCT/EP2019/085509

§ 371 Date: Jun. 15, 2021

(30) Foreign Application Priority Data
Dec. 19, 2018 (GB) 1820671.4

(51) Int. Cl. A01N 43/58; C07D 237/28; C07D 403/06

(52) U.S. Cl. CPC A01N 43/58; C07D 403/06; C07D 237/28

(57) ABSTRACT

Compounds of the formula (I) wherein the substituents are as defined in claim 1, useful as a pesticides, especially as herbicides.

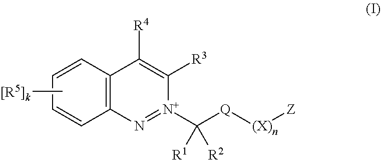

The present invention relates to herbicidally active cinnolinium derivatives, as well as to processes and intermediates used for the preparation of such derivatives. The invention further extends to herbicidal compositions comprising such derivatives, as well as to the use of such compounds and compositions for controlling undesirable plant growth: in particular the use for controlling weeds, in crops of useful plants.

Certain cinnolinium derivatives are known from U.S. Pat. No. 4,666,499 where they are stated to be useful for controlling unwanted plants.

The present invention is based on the finding that cinnolinium derivatives of formula (I) as defined herein, exhibit surprisingly good herbicidal activity. Thus, according to the present invention there is provided the use of a compound of formula (I) or an agronomically acceptable salt or zwitterionic species thereof, as a herbicide:

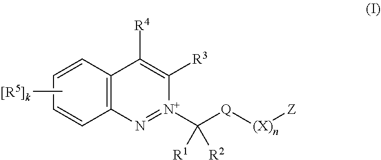

wherein:
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O;
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³ is selected from the group consisting of hydrogen, halogen, C₁-C₆alkyl, C₁-C₆haloalkyl and C₁-C₆alkoxy;
R⁴ is selected from the group consisting of hydrogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, which may be the same or different, and wherein the aforementioned CR¹R², Q and Z moieties may be attached at any position of said cycloalkyl, phenyl, heteroaryl or heterocyclyl moieties;
n is 0 or 1;
k is 0, 1, 2, 3 or 4;
when k is 1 or 2, each R⁵ is independently selected from the group consisting of halogen, nitro, cyano, —NH₂, —NR⁶R⁷, —OH, —OR⁷, —S(O)ᵣR¹², —NR⁶S(O)ᵣR¹², C₁-C₈alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
or,
when k is 3 or 4, each R⁵ is independently selected from the group consisting of halogen, C₁-C₆alkyl, C₁-C₆haloalkyl, C₁-C₆alkoxy and C₁-C₆haloalkoxy;
each R⁶ is independently selected from hydrogen and C₁-C₆alkyl;
R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a}; R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or R^{7b} and R^{7c} together with the nitrogen atom to which they are attached form a 4- to 6-membered heterocyclyl ring which optionally comprises one additional heteroatom individually selected from N, O and S; and
each R⁸ is independently selected from the group consisting of hydrogen and C₁-C₄alkyl;
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl, phenyl and benzyl, and wherein said phenyl or benzyl are optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2.

Certain compounds of formula (I) or agronomically acceptable salts or zwitterionic species thereof are known:
i) a compound of formula (I) selected from the group consisting of

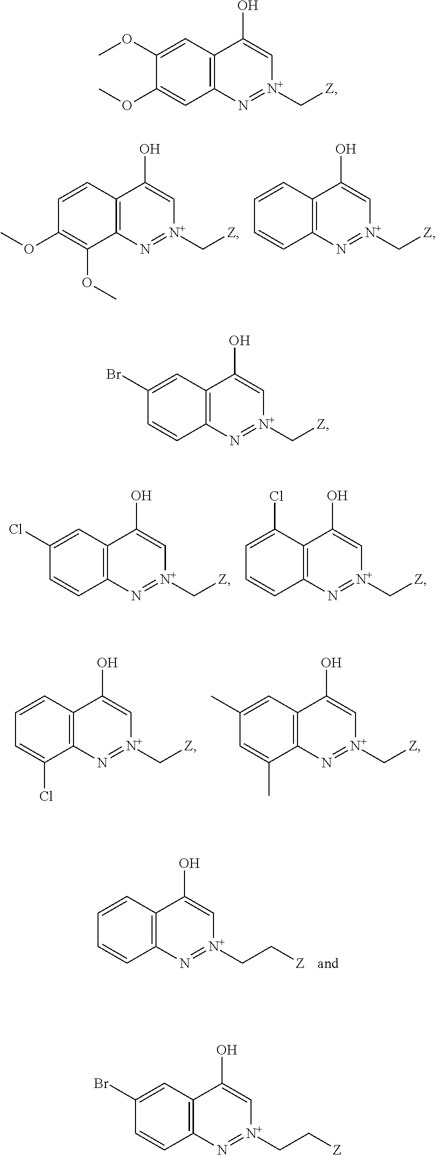

wherein Z is —CH₂OH, —C(O)OH or —C(O)OCH₂CH₃;
and
ii) the compound:

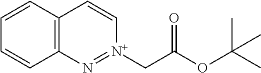 [SMILES: CC(C)(C)OC(=O)C[n+]1ccc2ccccc2n1]

tert-butyl 2-cinnolin-2-ium-2-ylacetate.

Thus in a second aspect of the invention there is provided a compound of formula (I) that is not i) or ii) listed above (or an agronomically acceptable salt or zwitterionic species thereof).

According to a third aspect of the invention, there is provided an agrochemical composition comprising a herbicidally effective amount of a compound of formula (I) and an agrochemically-acceptable diluent or carrier. Such an agricultural composition may further comprise at least one additional active ingredient.

According to a fourth aspect of the invention, there is provided a method of controlling or preventing undesirable plant growth, wherein a herbicidally effective amount of a compound of Formula (I), or a composition comprising this compound as active ingredient, is applied to the plants, to parts thereof or the locus thereof.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, amino means an —NH₂ group.

As used herein, nitro means an —NO₂ group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl (Me), ethyl (Et), n-propyl, 1-methylethyl (iso-propyl), n-butyl, and 1-dimethylethyl (f-butyl).

As used herein, the term “C₁-C₆alkoxy” refers to a radical of the formula —ORₐ where Rₐ is a C₁-C₆alkyl radical as generally defined above. C₁-C₄alkoxy is to be construed accordingly. Examples of C₁₋₄alkoxy include, but are not limited to, methoxy, ethoxy, propoxy, iso-propoxy and f-butoxy.

As used herein, the term “C₁-C₆haloalkyl” refers to a C₁-C₆alkyl radical as generally defined above substituted by one or more of the same or different halogen atoms. C₁-C₄haloalkyl is to be construed accordingly. Examples of C₁-C₆haloalkyl include, but are not limited to chloromethyl, fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. C₂-C₄alkenyl is to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical as generally defined above substituted by one or more of the same or different halogen atoms. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₂-C₄alkynyl is to be construed accordingly. Examples of C₂-C₆alkynyl include, but are not limited to, prop-1-ynyl, propargyl (prop-2-ynyl) and but-1-ynyl.

As used herein, the term “C₁-C₆haloalkoxy” refers to a C₁-C₆alkoxy group as defined above substituted by one or more of the same or different halogen atoms. C₁-C₄haloalkoxy is to be construed accordingly. Examples of C₁-C₆haloalkoxy include, but are not limited to, fluoromethoxy, difluoromethoxy, fluoroethoxy, trifluoromethoxy and trifluoroethoxy.

As used herein, the term “C₁-C₃haloalkoxyC₁-C₃alkyl” refers to a radical of the formula R_{b}—O—Rₐ— where R_{b} is a C₁-C₃haloalkyl radical as generally defined above, and Rₐ is a C₁-C₃alkylene radical as generally defined above.

As used herein, the term “C₁-C₃alkoxyC₁-C₃alkyl” refers to a radical of the formula R_{b}—O—Rₐ— where R_{b} is a C₁-C₃alkyl radical as generally defined above, and Rₐ is a C₁-C₃alkylene radical as generally defined above.

As used herein, the term “C₃-C₆alkenyloxy” refers to a radical of the formula —ORₐ where Rₐ is a C₃-C₆alkenyl radical as generally defined above.

As used herein, the term “C₃-C₆alkynyloxy” refers to a radical of the formula —ORₐ where Rₐ is a C₃-C₆alkynyl radical as generally defined above.

As used herein, the term “hydroxyC₁-C₆alkyl” refers to a C₁-C₆alkyl radical as generally defined above substituted by one or more hydroxy groups.

As used herein, the term “C₁-C₆alkylcarbonyl” refers to a radical of the formula —C(O)Rₐ where Rₐ is a C₁-C₆alkyl radical as generally defined above.

As used herein, the term “C₁-C₆alkoxycarbonyl” refers to a radical of the formula —C(O)ORₐ where Rₐ is a C₁-C₆alkyl radical as generally defined above.

As used herein, the term “aminocarbonyl” refers to a radical of the formula —C(O)NH₂.

As used herein, the term “C₁-C₆alkylaminocarbonyl” refers to a radical of the formula —C(O)NHRₐ where Rₐ is a C₁-C₆alkyl radical as generally defined above.

As used herein, the term “di-C₁-C₆alkylaminocarbonyl” refers to a radical of the formula —C(O)NRₐ(Rₐ) where each Rₐ is independently a C₁-C₆alkyl radical as generally defined above.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to, cyclopropyl, cyclobutyl, cyclopentyl and cyclohexyl.

As used herein, the term “C₃-C₆halocycloalkyl” refers to a C₃-C₆cycloalkyl radical as generally defined above substituted by one or more of the same or different halogen atoms. C₃-C₄halocycloalkyl is to be construed accordingly.

As used herein, the term “C₃-C₆cycloalkoxy” refers to a radical of the formula —ORₐ where Rₐ is a C₃-C₆cycloalkyl radical as generally defined above.

As used herein, except where explicitly stated otherwise, the term “heteroaryl” refers to a 5- or 6-membered monocyclic aromatic ring which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heterocyclyl” or “heterocyclic” refers to a stable 3- to 6-membered non-aromatic monocyclic ring radical which comprises 1, 2, or 3 heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or δ-lactamyl.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

For example a compound of formula (I) wherein Z comprises an acidic proton, may exist as a zwitterion, a compound of formula (I-I), or as an agronomically acceptable salt, a compound of formula (I-II) as shown below:

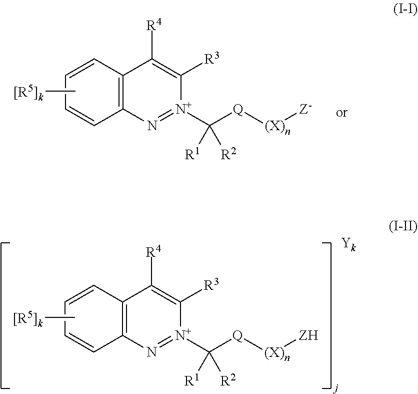

wherein, Y represents an agronomically acceptable anion and j and k represent integers that may be selected from 1, 2 or 3, dependent upon the charge of the respective anion Y.

A compound of formula (I) may also exist as an agronomically acceptable salt of a zwitterion, a compound of formula (I-III) as shown below:

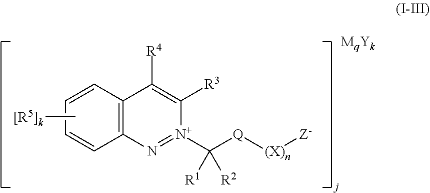

wherein, Y represents an agronomically acceptable anion, M represents an agronomically acceptable cation (in addition to the cinnolinium cation) and the integers j, k and q may be selected from 1, 2 or 3, dependent upon the charge of the respective anion Y and respective cation M.

Thus where a compound of formula (I) is drawn in protonated form herein, the skilled person would appreciate that it could equally be represented in unprotonated or salt form with one or more relevant counterions.

In one embodiment of the invention there is provided a compound of formula (I-II) wherein k is 2, j is 1 and Y is selected from the group consisting of halogen, trifluoroacetate and pentafluoropropionate.

Suitable agronomically acceptable salts of the present invention, represented by an anion Y, include but are not limited chloride, bromide, iodide, fluoride, 2-naphthalenesulfonate, acetate, adipate, methoxide, ethoxide, propoxide, butoxide, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydrogen sulfate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, pentafluoropropionate, perchlorate, phosphate, propionate, propylsulfate, propylsulfonate, succinate, sulfate, tartrate, tosylate, tridecylate, triflate, trifluoroacetate, undecylinate and valerate.

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

Preferred compounds of formula (I), wherein Z comprises an acidic proton, can be represented as either (I-I) or (I-II). For compounds of formula (I-II) emphasis is given to salts when Y is chloride, bromide, iodide, hydroxide, bicarbonate, acetate, pentafluoropropionate, triflate, trifluoroacetate, methylsulfate, tosylate and nitrate, wherein j and k are 1. Preferably, Y is chloride, bromide, iodide, hydroxide, bicarbonate, acetate, trifluoroacetate, methylsulfate, tosylate and nitrate, wherein j and k are 1. For compounds of formula (I-II) emphasis is also given to salts when Y is carbonate and sulfate, wherein j is 2 and k is 1, and when Y is phosphate, wherein j is 3 and k is 1.

Where appropriate compounds of formula (I) may also be in the form of (and/or be used as) an N-oxide.

Compounds of formula (I) wherein m is 0 and n is 0 may be represented by a compound of formula (I-Ia) as shown below:

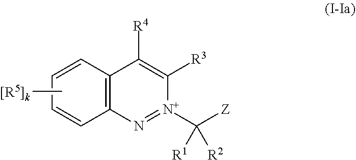

wherein k, R¹, R², R³, R⁴, R⁵ and Z are as defined for compounds of formula (I).

Compounds of formula (I) wherein m is 1 and n is 0 may be represented by a compound of formula (I-Ib) as shown below:

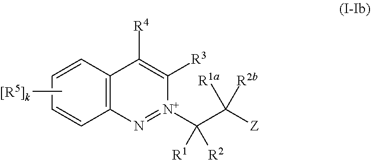

wherein k, R¹, R², R^{1a}, R^{2b}, R³, R⁴, R⁵ and Z are as defined for compounds of formula (I).

Compounds of formula (I) wherein m is 2 and n is 0 may be represented by a compound of formula (I-Ic) as shown below:

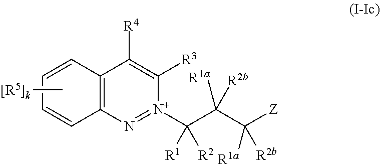

wherein k, R¹, R², R^{1a}, R^{2b}, R³, R⁴, R⁵ and Z are as defined for compounds of formula (I).

Compounds of formula (I) wherein m is 3 and n is 0 may be represented by a compound of formula (I-Id) as shown below:

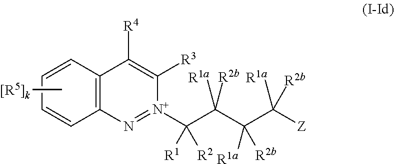

wherein k, R¹, R², R^{1a}, R^{2b}, R³, R⁴, R⁵ and Z are as defined for compounds of formula (I).

The following list provides definitions, including preferred definitions, for substituents k, n, m, r, Q, X, Z, R¹, R², R^{1a}, R^{2b}, R³, R⁴, R⁵, R⁶, R⁷, R^{7a}, R^{7b}, R^{7c}, R⁸, R⁹, R¹⁰, R¹¹, R¹², R¹³, R¹⁴, R¹⁵, R^{15a}, R¹⁶, R¹⁷ and R¹⁸ with reference to the compounds of formula (I) according to the invention. For any one of these substituents, any of the definitions given below may be combined with any definition of any other substituent given below or elsewhere in this document.

R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵. Preferably, R¹ is selected from the group consisting of hydrogen, halogen, C₁-C₆alkyl, C₁-C₆fluoroalkyl, —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵. More preferably, R¹ is selected from the group consisting of hydrogen, halogen, C₁-C₆alkyl, C₁-C₆fluoroalkyl, —OR⁷ and —N(R^{7a})₂. Even more preferably, R¹ is selected from the group consisting of hydrogen, C₁-C₆alkyl, —OR⁷ and —N(R^{7a})₂. Even more preferably still, R¹ is hydrogen or C₁-C₆alkyl. Yet even more preferably still, R¹ is hydrogen or C₁-C₃alkyl (preferably methyl). Most preferably R¹ is hydrogen.

R² is selected from the group consisting of hydrogen, halogen, C₁-C₆alkyl and C₁-C₆haloalkyl. Preferably, R² is selected from the group consisting of hydrogen, halogen, C₁-C₆alkyl and C₁-C₆fluoroalkyl. More preferably, R² is hydrogen or C₁-C₆alkyl. Even more preferably, R² is hydrogen or C₁-C₃alkyl (preferably methyl). Most preferably R² is hydrogen.

Wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl. Preferably, when R¹ is selected from the group consisting of —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵, R² is selected from the group consisting of hydrogen and methyl.

Alternatively, R¹ and R² together with the carbon atom to which they are attached form a C₃-C₆cycloalkyl ring or a 3- to 6-membered heterocyclyl, which comprises 1 or 2 heteroatoms individually selected from N and O. Preferably, R¹ and R² together with the carbon atom to which they are attached form a C₃-C₆cycloalkyl ring. More preferably, R¹ and R² together with the carbon atom to which they are attached form a cyclopropyl ring.

In one embodiment R¹ and R² are independently selected from the group consisting of hydrogen and C₁-C₃alkyl.

In another embodiment R¹ and R² are hydrogen.

In another embodiment R¹ is methyl and R² is hydrogen.

In another embodiment R¹ is methyl and R² is methyl.

Q is (CR^{1a}R^{2b})ₘ.

m is 0, 1, 2 or 3. Preferably, m is 0, 1 or 2. More preferably, m is 1 or 2. Most preferably, m is 1.

Each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵. Preferably, each R^{1a} and R^{2b} are independently selected from the group consisting of hydrogen, halogen, C₁-C₆alkyl, C₁-C₆fluoroalkyl, —OH, —NH₂ and —NHR⁷. More preferably, each R^{1a} and R^{2b} are independently selected from the group consisting of hydrogen, C₁-C₆alkyl, —OH and —NH₂. Even more preferably, each R^{1a} and R^{2b} are independently selected from the group consisting of hydrogen, methyl, —OH and —NH₂. Even more preferably still, each R^{1a} and R^{2b} are independently selected from the group consisting of hydrogen and methyl. Most preferably R^{1a} and R^{2b} are hydrogen.

Alternatively, each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O. Preferably, each R^{1a} and R^{2b} together with the carbon atom to which they are attached form a C₃-C₆cycloalkyl ring. More preferably, each R^{1a} and R^{2b} together with the carbon atom to which they are attached form a cyclopropyl ring.

R³ is selected from the group consisting of hydrogen, halogen, C₁-C₆alkyl, C₁-C₆haloalkyl and C₁-C₆alkoxy. Preferably, R³ is selected from the group consisting of hydrogen, halogen and C₁-C₆alkyl. More preferably, R³ is selected from the group consisting of hydrogen, halogen and C₁-C₃alkyl. Even more preferably, R³ is selected from the group consisting of hydrogen, chloro and methyl. Even more preferably still, R³ is hydrogen or methyl. Most preferably R³ is hydrogen.

R⁴ is selected from the group consisting of hydrogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different.

Preferably, R⁴ is selected from the group consisting of hydrogen, —NH₂, —NR⁶R⁷, —OH, —OR⁷, —S(O)ᵣR¹², C₁-C₃alkyl, C₁-C₃haloalkyl, C₃-C₆cycloalkyl, C₃-C₆halocycloalkyl, C₂-C₄alkenyl, C₂-C₄alkynyl, C₁-C₃alkoxyC₁-C₃alkyl-, hydroxyC₁-C₃alkyl-, C₁-C₃haloalkoxy, C₁-C₃haloalkoxyC₁-C₃alkyl-, C₁-C₃alkoxycarbonyl, C₁-C₃alkylcarbonyl, C₁-C₃alkylaminocarbonyl, di-C₁-C₃alkylaminocarbonyl or phenyl and wherein said phenyl moiety is optionally substituted by 1, 2 or 3 R⁹ substituents, which may be the same or different.

More preferably, R⁴ is selected from the group consisting of hydrogen, —NH₂, —NR⁶R⁷, —OR⁷, —S(O)ᵣR¹², C₁-C₃alkyl, C₁-C₃haloalkyl, C₃-C₆cycloalkyl, C₂-C₄alkenyl, C₂-C₄alkynyl, C₁-C₃haloalkoxy, C₁-C₃alkylaminocarbonyl and phenyl, wherein said phenyl is optionally substituted by 1, 2 or 3 R⁹ substituents, which may be the same or different.

Further more preferably, R⁴ is selected from the group consisting of hydrogen, —NH₂, —NR⁶R⁷, —OR⁷, —S(O)ᵣR¹², C₁-C₃alkyl, C₁-C₃haloalkyl, C₃-C₆cycloalkyl, C₂-C₄alkynyl, C₁-C₃alkylaminocarbonyl and phenyl, wherein said phenyl is optionally substituted by 1, 2 or 3 R⁹ substituents, which may be the same or different.

Even further more preferably, R⁴ is selected from the group consisting of hydrogen, —OR⁷, —S(O)ᵣR¹², C₁-C₃alkyl, C₁-C₃haloalkyl, C₃-C₆cycloalkyl, C₂-C₄alkynyl, C₁-C₃alkylaminocarbonyl and phenyl, wherein said phenyl is optionally substituted by 1, 2 or 3 R⁹ substituents, which may be the same or different.

Even further more preferably still, R⁴ is selected from the group consisting of hydrogen, —OMe, —SMe, methyl, dichloromethyl, trichloromethyl, cyclopropyl, prop-1-ynyl, methylaminocarbonyl and phenyl.

Yet even further more preferably still, R⁴ is hydrogen or methyl. Most preferably, R⁴ is hydrogen,

k is 0, 1, 2, 3 or 4.

Preferably k is 0, 1 or 2. More preferably k is 0 or 1.

In one embodiment k is 0. In another embodiment k is 1.

When k is 1 or 2, each R⁵ is independently selected from the group consisting of halogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different.

Preferably when k is 1 or 2, each R⁵ is independently selected from the group consisting of halogen, nitro, cyano, —NH₂, —NR⁶R⁷, —OH, —OR⁷, C₁-C₆alkyl, C₁-C₆haloalkyl, C₃-C₆cycloalkyl, C₃-C₆cycloalkoxy, C₂-C₆alkenyl, C₂-C₆haloalkenyl, C₂-C₆alkynyl, C₁-C₆haloalkoxy, C₁-C₃haloalkoxyC₁-C₃alkyl-, C₁-C₆alkoxycarbonyl, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different.

More preferably, when k is 1 or 2, each R⁵ is independently selected from the group consisting of halogen, cyano, —NH₂, —NR⁶R⁷, —OH, —OR⁷, C₁-C₃alkyl, C₁-C₃haloalkyl, C₃-C₆cycloalkyl, C₁-C₃haloalkoxy, C₂-C₄alkenyl, C₂-C₄alkynyl, C₁-C₃alkoxycarbonyl, C₁-C₃alkylaminocarbonyl, di-C₁-C₃alkylaminocarbonyl and phenyl, wherein said phenyl is optionally substituted by 1, 2 or 3 R⁹ substituents, which may be the same or different.

Further more preferably, when k is 1 or 2, each R⁵ is independently selected from the group consisting of halogen, cyano, —NR⁶R⁷, —OR⁷, C₁-C₃alkyl, C₁-C₃haloalkyl, C₁-C₃alkoxycarbonyl, C₁-C₃alkylaminocarbonyl, di-C₁-C₃alkylaminocarbonyl and phenyl.

Further more preferably still, when k is 1 or 2, each R⁵ is independently selected from the group consisting of chloro, fluoro, bromo, iodo, cyano, —NHC(O)Me, —OMe, methyl, trifluoromethyl, methoxycarbonyl, di-methylaminocarbonyl and phenyl.

Yet further more preferably still, when k is 1 or 2, each R⁵ is independently selected from the group consisting of chloro, fluoro, bromo, iodo, —NHC(O)Me, —OMe, methyl and di-methylaminocarbonyl.

Alternatively, when k is 3 or 4, each R⁵ is independently selected from the group consisting of halogen, C₁-C₆alkyl, C₁-C₆haloalkyl, C₁-C₆alkoxy and C₁-C₆haloalkoxy. Preferably each R⁵ is independently selected from the group consisting of chloro, fluoro, bromo, iodo, methoxy, methyl and trifluoromethyl. More preferably each R⁵ is independently selected from the group consisting of chloro, fluoro, methoxy and methyl. Even more preferably each R⁵ is independently selected from the group consisting of chloro, fluoro and methyl. Most preferably each R⁵ is methyl.

Each R⁶ is independently selected from hydrogen and C₁-C₆alkyl. Preferably, each R⁶ is independently selected from hydrogen and methyl.

R⁷ is independently selected from the group consisting of C₁-C₆alkyl, —S(O)₂R¹⁵, —C(O)R¹⁵, —C(O)OR¹⁵ and —C(O)NR¹⁶R¹⁷. Preferably, each R⁷ is independently selected from the group consisting of C₁-C₆alkyl, —C(O)R¹⁵ and —C(O)NR¹⁶R¹⁷. More preferably, each R⁷ is C₁-C₆alkyl or —C(O)R¹⁵ (for example, —C(O)Me). Even more preferably, each R⁷ is C₁-C₆alkyl. Most preferably, each R⁷ is methyl.

Each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a}. Preferably, each R^{7a} is independently —C(O)R¹⁵ or —C(O)NR¹⁶R¹⁷.

R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different. Preferably, R^{7b} and R^{7c} are independently selected from the group consisting of C₁-C₆alkyl, —C(O)R¹⁵ and —C(O)NR¹⁶R¹⁷. More preferably, R^{7b} and R^{7c} are C₁-C₆alkyl. Most preferably, R^{7b} and R^{7c} are methyl.

Alternatively, R^{7b} and R^{7c} together with the nitrogen atom to which they are attached form a 4- to 6-membered heterocyclyl ring which optionally comprises one additional heteroatom individually selected from N, O and S. Preferably, R^{7b} and R^{7c} together with the nitrogen atom to which they are attached form a 5- to 6-membered heterocyclyl ring which optionally comprises one additional heteroatom individually selected from N and O. More preferably, R^{7b} and R^{7c} together with the nitrogen atom to which they are attached form an pyrrolidyl, oxazolidinyl, imidazolidinyl, piperidyl, piperazinyl or morpholinyl group.

Each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy. Preferably, each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy. More preferably, each R⁹ is independently selected from the group consisting of halogen, C₁-C₄alkyl, C₁-C₄alkoxy and C₁-C₄haloalkyl. Even more preferably, each R⁹ is independently selected from the group consisting of halogen and C₁-C₄alkyl.

Each R⁸ is independently selected from the group consisting of hydrogen and C₁-C₄alkyl. Preferably, each R⁸ is independently selected from the group consisting of hydrogen and methyl. More preferably, each R⁸ is methyl.

X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, which may be the same or different, and wherein the aforementioned CR¹R², Q and Z moieties may be attached at any position of said cycloalkyl, phenyl, heteroaryl or heterocyclyl moieties.

Preferably, X is selected from the group consisting of phenyl and a 4- to 6-membered heterocyclyl, which comprises 1 or 2 heteroatoms individually selected from N and O, and wherein said phenyl or heterocyclyl moieties are optionally substituted by 1 or 2 R⁹ substituents, which may be the same or different, and wherein the aforementioned CR¹R², Q and Z moieties may be attached at any position of said phenyl or heterocyclyl moieties.

More preferably, X is phenyl or a 5-membered heterocyclyl, which comprises 1 or 2 heteroatoms individually selected from N and O, and wherein said phenyl and heterocyclyl moieties are optionally substituted by 1 or 2 R⁹ substituents, which may be the same or different, and wherein the aforementioned CR¹R², Q and Z moieties may be attached at any position of said phenyl or heterocyclyl moieties.

In one embodiment, X is a 5-membered heterocyclyl, which comprises 1 heteroatom, wherein said heteroatom is N, and wherein the aforementioned CR¹R², Q and Z moieties may be attached at any position of said heterocyclyl moiety. Preferably, X is a 5-membered heterocyclyl, which comprises 1 heteroatom, wherein said heteroatom is N, and wherein the aforementioned CR¹R² and Q moieties are attached adjacent to the N atom and the Z moiety is attached to the N atom.

In another embodiment, X is phenyl optionally substituted by 1 or 2 R⁹ substituents, which may be the same or different, and wherein the aforementioned CR¹R², Q and Z moieties may be attached at any position of said phenyl moiety. Preferably, X is phenyl and the aforementioned CR¹R² and Q moieties are attached in a position para to the Z moiety.

n is 0 or 1. Preferably, n is 0.

Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole.

Preferably, Z is selected from the group consisting of —C(O)OR¹⁰, —CH₂OH, —C(O)NHOR¹¹, —OC(O)NHOR¹¹, —NR⁶C(O)NHOR¹¹, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHC(O)R¹⁸, —S(O)₂NHS(O)₂R¹², —OS(O)₂NHS(O)₂R¹², —OS(O)₂NHC(O)R¹⁸, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole.

More preferably, Z is selected from the group consisting of —C(O)OR¹⁰, —CH₂OH, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰, —P(O)(R¹³)(OR¹⁰) and tetrazole.

Even more preferably Z is selected from the group consisting of —C(O)OH, —C(O)OCH₃, —C(O)OCH(CH₃)₂, —C(O)OC(CH₃)₃, —CH₂OH, —C(O)NHOCH₃, —C(O)NHS(O)₂CH₃, —C(O)NHS(O)₂N(CH₃)₂, —S(O)₂OH, —OS(O)₂OH, —NHS(O)₂OH, —NHS(O)₂CF₃, —P(O)(OH)(OH), —P(O)(OH)(OCH₃), —P(O)(OCH₃)(OCH₃), —P(O)(OH)(OCH₂CH₃), —P(O)(OCH₂CH₃)(OCH₂CH₃) and tetrazole.

Even more preferably still Z is selected from the group consisting of —C(O)OH, —C(O)NHS(O)₂CH₃, —S(O)₂OH, —OS(O)₂OH and —NHS(O)₂OH.

Most preferably Z is —C(O)OH or —S(O)₂OH.

R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different. Preferably, R¹⁰ is selected from the group consisting of hydrogen, C₁-C₆alkyl, phenyl and benzyl. More preferably, R¹⁰ is selected from the group consisting of hydrogen and C₁-C₆alkyl. Most preferably, R¹⁰ is hydrogen.

R¹¹ is selected from the group consisting of hydrogen, C₁-C₆alkyl and phenyl, and wherein said phenyl is optionally substituted by 1, 2 or 3 R⁹ substituents, which may be the same or different. Preferably, R¹¹ is selected from the group consisting of hydrogen, C₁-C₆alkyl and phenyl. More preferably, R¹¹ is selected from the group consisting of hydrogen and C₁-C₆alkyl. Even more preferably, R¹¹ is C₁-C₆alkyl. Most preferably, R¹¹ is methyl.

R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different. Preferably, R¹² is selected from the group consisting of C₁-C₆alkyl, C₁-C₆haloalkyl, C₁-C₆alkoxy, —OH, —N(R⁶)₂ and phenyl. More preferably, R¹² is selected from the group consisting of C₁-C₆alkyl, C₁-C₆haloalkyl and —N(R⁶)₂. Even more preferably, R¹² is selected from the group consisting of methyl, —N(Me)₂ and trifluoromethyl. Most preferably, R¹² is methyl.

R¹³ is selected from the group consisting of —OH, C₁-C₆alkyl, C₁-C₆alkoxy and phenyl. Preferably R¹³ is selected from the group consisting of —OH, C₁-C₆alkyl and C₁-C₆alkoxy. More preferably, R¹³ is selected from the group consisting of —OH and C₁-C₆alkoxy. Even more preferably, R¹³ is selected from the group consisting of —OH, methoxy and ethoxy. Most preferably, R¹³ is —OH.

R¹⁴ is C₁-C₆haloalkyl. Preferably, R¹⁴ is trifluoromethyl.

R¹⁵ is selected from the group consisting of C₁-C₆alkyl, phenyl and benzyl, and wherein said phenyl or benzyl are optionally substituted by 1, 2 or 3 R⁹ substituents, which may be the same or different. Preferably, R¹⁵ is selected from the group consisting of C₁-C₆alkyl, phenyl and benzyl. More preferably, R¹⁵ is C₁-C₆alkyl. Most preferably R¹⁵ is methyl.

R^{15a} is phenyl, wherein said phenyl is optionally substituted by 1, 2 or 3 R⁹ substituents, which may be the same or different. Preferably, R^{15a} is phenyl optionally substituted by 1 R⁹ substituent. More preferably, R^{15a} is phenyl.

R¹⁶ and R¹⁷ are independently selected from the group consisting of hydrogen and C₁-C₆alkyl. Preferably, R¹⁶ and R¹⁷ are independently selected from the group consisting of hydrogen and methyl.

Alternatively, R¹⁶ and R¹⁷ together with the nitrogen atom to which they are attached form a 4- to 6-membered heterocyclyl ring which optionally comprises one additional heteroatom individually selected from N, O and S. Preferably, R¹⁶ and R¹⁷ together with the nitrogen atom to which they are attached form a 5- to 6-membered heterocyclyl ring which optionally comprises one additional heteroatom individually selected from N and O. More preferably, R¹⁶ and R¹⁷ together with the nitrogen atom to which they are attached form an pyrrolidyl, oxazolidinyl, imidazolidinyl, piperidyl, piperazinyl or morpholinyl group.

R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different. Preferably, R¹⁸ is selected from the group consisting of hydrogen, C₁-C₆alkyl, C₁-C₆haloalkyl, C₁-C₆alkoxy, —N(R⁶)₂ and phenyl. More preferably, R¹⁸ is selected from the group consisting of hydrogen, C₁-C₆alkyl and C₁-C₆haloalkyl. Further more preferably, R¹⁸ is selected from the group consisting of C₁-C₆alkyl and C₁-C₆haloalkyl. Most preferably, R¹⁸ is methyl or trifluoromethyl.

r is 0, 1 or 2. Preferably, r is 0 or 2.

In a set of preferred embodiments, in a compound according to formula (I) of the invention,

R¹ is hydrogen or methyl;
R² is hydrogen or methyl;
Q is (CR^{1a}R^{2b})ₘ;
m is 0, 1 or 2;
R^{1a} and R^{2b} are independently selected from the group consisting of hydrogen, C₁-C₆alkyl, —OH and —NH₂;
R³ is independently selected from the group consisting of hydrogen, halogen and C₁-C₃alkyl;
R⁴ is independently selected from the group consisting of hydrogen, —OR⁷, —S(O)ᵣR¹², C₁-C₃alkyl, C₁-C₃haloalkyl, C₃-C₆cycloalkyl, C₂-C₄alkynyl, C₁-C₃alkylaminocarbonyl and phenyl, wherein said phenyl is optionally substituted by 1, 2 or 3 R⁹ substituents, which may be the same or different;
k is 0, 1 or 2;
each R⁵ is independently selected from the group consisting of halogen, cyano, —NR⁶R⁷, —OR⁷, C₁-C₃alkyl, C₁-C₃haloalkyl, C₁-C₃alkoxycarbonyl, C₁-C₃alkylaminocarbonyl, di-C₁-C₃alkylaminocarbonyl and phenyl;
each R⁶ is independently selected from hydrogen and methyl;
each R⁷ is C₁-C₆alkyl or —C(O)R¹⁵ (preferably —C(O)Me);
n is 0;
each R⁹ is independently selected from the group consisting of halogen and C₁-C₄alkyl;
Z is selected from the group consisting of —C(O)OR¹⁰, —CH₂OH, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰, —P(O)(R¹³)(OR¹⁰) and tetrazole;
R¹⁰ is selected from the group consisting of hydrogen and C₁-C₆alkyl;
R¹¹ is C₁-C₆alkyl;
R¹² is selected from the group consisting of C₁-C₆alkyl, C₁-C₆haloalkyl and —N(R⁶)₂;
R¹³ is selected from the group consisting of —OH and C₁-C₆alkoxy;
R¹⁴ is trifluoromethyl; and
r is 0 or 2.

More preferably,

R¹ is hydrogen;
R² is hydrogen;
Q is (CR^{1a}R^{2b})ₘ;
m is 0, 1 or 2;
each R^{1a} and R^{2b} are independently selected from the group consisting of hydrogen, methyl, —OH and —NH₂;
R³ is independently selected from the group consisting of hydrogen, chloro and methyl;
R⁴ is selected from the group consisting of hydrogen, —OMe, —SMe, methyl, dichloromethyl, trichloromethyl, cyclopropyl, prop-1-ynyl, methylaminocarbonyl and phenyl;
k is 0, 1 or 2;
each R⁵ is independently selected from the group consisting of chloro, fluoro, bromo, iodo, cyano, —NHC(O)Me, —OMe, methyl, trifluoromethyl, methoxycarbonyl, di-methylaminocarbonyl and phenyl;
n is 0; and
Z is selected from the group consisting of —C(O)OH, —C(O)OCH₃, —C(O)OCH(CH₃)₂, —C(O)OC(CH₃)₃, —CH₂OH, —C(O)NHOCH₃, —C(O)NHS(O)₂CH₃, —C(O)NHS(O)₂N(CH₃)₂, —S(O)₂OH, —OS(O)₂OH, —NHS(O)₂OH, —NHS(O)₂CF₃, —P(O)(OH)(OH), —P(O)(OH)(OCH₃), —P(O)(OCH₃)(OCH₃), —P(O)(OH)(OCH₂CH₃), —P(O)(OCH₂CH₃)(OCH₂CH₃) and tetrazole.

In a further set of preferred embodiments, the compound according to formula (I) is selected from a compound of formula (I-a), (I-b) or (I-c),

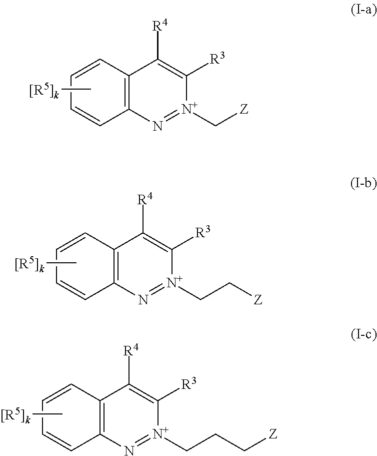

wherein in a compound of formula (I-a), (I-b) or (I-c)
k is 0 or 1
R³ is hydrogen;
R⁴ is selected from the group consisting of hydrogen, —OMe and methyl;
k is 0 or 1;
each R⁵ is independently selected from the group consisting of chloro, fluoro, —OMe, methyl and trifluoromethyl;
Z is selected from the group consisting of —C(O)OH, —C(O)NHS(O)₂CH₃, —S(O)₂OH, —OS(O)₂OH and —NHS(O)₂OH.

In a further more preferred set of embodiments, the compound according to formula (I) is selected from a compound of formula (I-d), (I-e) or (I-f),

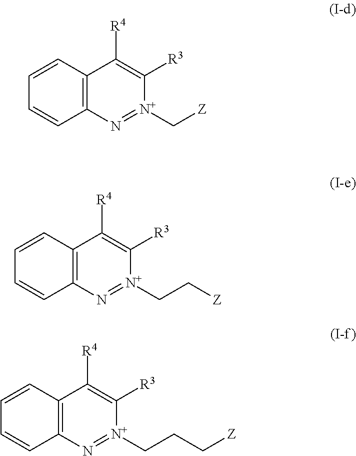

wherein in a compound of formula (I-d), (I-e) or (I-f)
R³ is hydrogen;
R⁴ is hydrogen;
Z is selected from the group consisting of —C(O)OH, —C(O)NHS(O)₂CH₃, —S(O)₂OH, —OS(O)₂OH and —NHS(O)₂OH.

In one set of embodiments, the compound according to formula (I) is selected from a compound A1 to A123 listed in Table A.

It should be understood that compounds of formula (I) may exist/be manufactured in ‘procidal form’, wherein they comprise a group ‘G’. Such compounds are referred to herein as compounds of formula (I-IV).

G is a group which may be removed in a plant by any appropriate mechanism including, but not limited to, metabolism and chemical degradation to give a compound of formula (I-I), (I-II) or (I-III) wherein Z contains an acidic proton, for example see the scheme below:

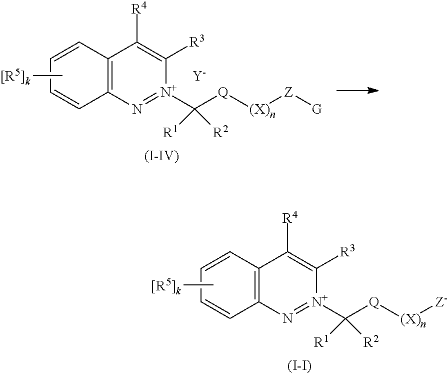

Whilst such G groups may be considered as ‘procidal’, and thus yield active herbicidal compounds once removed, compounds comprising such groups may also exhibit herbicidal activity in their own right. In such cases in a compound of formula (I-IV), Z-G may include but is not limited to, any one of (G1) to (G7) below and E indicates the point of attachment to the remaining part of a compound of formula (I):

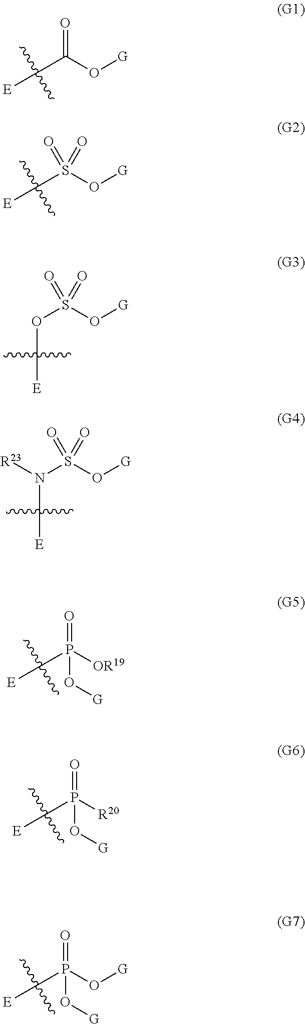

In embodiments where Z-G is (G1) to (G7), G, R¹⁹, R²⁰, R²¹, R²² and R²³ are defined as follows:

G is C₁-C₆alkyl, C₂-C₆alkenyl, C₂-C₆alkynyl, —C(R²¹R²²)OC(O)R¹⁹, phenyl or phenyl-C₁-C₄alkyl-, wherein said phenyl moiety is optionally substituted by 1 to 5 substituents independently selected from halo, cyano, nitro, C₁-C₆alkyl, C₁-C₆haloalkyl or C₁-C₆alkoxy.

R¹⁹ is C₁-C₆alkyl or phenyl,
R²⁰ is hydroxy, C₁-C₆alkyl, C₁-C₆alkoxy or phenyl,
R²¹ is hydrogen or methyl,
R²² is hydrogen or methyl,
R²³ is hydrogen or C₁-C₆alkyl.

The compounds in Tables 1 to 22 below illustrate the compounds of the invention. The skilled person would understand that the compounds of formula (I) may exist as an agronomically acceptable salt, a zwitterion or an agronomically acceptable salt of a zwitterion as described hereinbefore.

TABLE 1
This table discloses 53 specific compounds of the formula (T-1):
(T-1)

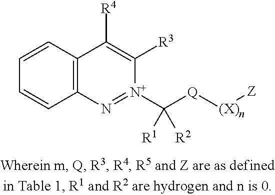

| Compound | | | | | | |
|---|---|---|---|---|---|---|
| number | R³ | R⁴ | R⁵ | Z | m | Q |
| 1.001 | H | H | H | —C(O)OH | 0 | — |
| 1.002 | H | H | H | —C(O)OMe | 0 | — |
| 1.003 | H | H | H | —C(O)NHOMe | 0 | — |
| 1.004 | H | H | H | —OC(O)NHOMe | 0 | — |
| 1.005 | H | H | H | —NHC(O)NHOMe | 0 | — |
| 1.006 | H | H | H | —NMeC(O)NHOMe | 0 | — |
| 1.007 | H | H | H | —C(O)NHS(O)₂Me | 0 | — |
| 1.008 | H | H | H | —OC(O)NHS(O)₂Me | 0 | — |
| 1.009 | H | H | H | —NHC(O)NHS(O)₂Me | 0 | — |
| 1.010 | H | H | H | —NMeC(O)NHS(O)₂Me | 0 | — |
| 1.011 | H | H | H | —S(O)₂OH | 0 | — |
| 1.012 | H | H | H | —OS(O)₂OH | 0 | — |
| 1.013 | H | H | H | —NHS(O)₂OH | 0 | — |
| 1.014 | H | H | H | —NMeS(O)₂OH | 0 | — |
| 1.015 | H | H | H | —S(O)OH | 0 | — |
| 1.016 | H | H | H | —OS(O)OH | 0 | — |
| 1.017 | H | H | H | —NHS(O)OH | 0 | — |
| 1.018 | H | H | H | —NMeS(O)OH | 0 | — |
| 1.019 | H | H | H | —NHS(O)₂CF₃ | 0 | — |
| 1.020 | H | H | H | —S(O)₂NHC(O)Me | 0 | — |
| 1.021 | H | H | H | —OS(O)₂NHC(O)Me | 0 | — |
| 1.022 | H | H | H | —NHS(O)₂NHC(O)Me | 0 | — |
| 1.023 | H | H | H | —NMeS(O)₂NHC(O)Me | 0 | — |
| 1.024 | H | H | H | —P(O)(OH)(OMe) | 0 | — |
| 1.025 | H | H | H | —P(O)(OH)(OH) | 0 | — |
| 1.026 | H | H | H | —OP(O)(OH)(OMe) | 0 | — |
| 1.027 | H | H | H | —OP(O)(OH)(OH) | 0 | — |
| 1.028 | H | H | H | —NHP(O)(OH)(OMe) | 0 | — |
| 1.029 | H | H | H | —NHP(O)(OH)(OH) | 0 | — |
| 1.030 | H | H | H | —NMeP(O)(OH)(OMe) | 0 | — |
| 1.031 | H | H | H | —NMeP(O)(OH)(OH) | 0 | — |
| 1.032 | H | H | H | -tetrazole | 0 | — |
| 1.033 | H | H | H | —S(O)₂OH | 1 | CH(NH₂) |
| 1.033 | H | H | H | —C(O)OH | 1 | CH(NH₂) |
| 1.035 | H | H | H | —S(O)₂OH | 2 | CH(OH)CH₂ |
| 1.036 | H | H | H | —C(O)OH | 2 | CH(OH)CH₂ |
| 1.037 | H | H | H | —S(O)₂OH | 1 | CH(OH) |
| 1.038 | H | H | H | —C(O)OH | 1 | CH(OH) |
| 1.039 | H | H | H | —C(O)NHCN | 0 | — |
| 1.040 | H | H | H | —OC(O)NHCN | 0 | — |
| 1.041 | H | H | H | —NHC(O)NHCN | 0 | — |
| 1.042 | H | H | H | —NMeC(O)NHCN | 0 | — |
| 1.043 | H | H | H | —S(O)₂NHCN | 0 | — |
| 1.044 | H | H | H | —OS(O)₂NHCN | 0 | — |
| 1.045 | H | H | H | —NHS(O)₂NHCN | 0 | — |
| 1.046 | H | H | H | —NMeS(O)₂NHCN | 0 | — |
| 1.047 | H | H | H | —S(O)₂NHS(O)₂Me | 0 | — |
| 1.048 | H | H | H | —OS(O)₂NHS(O)₂Me | 0 | — |
| 1.049 | H | H | H | —NHS(O)₂NHS(O)₂Me | 0 | — |
| 1.050 | H | H | H | —NMeS(O)₂NHS(O)₂Me | 0 | — |
| 1.051 | H | H | H | —P(O)H(OH) | 0 | — |
| 1.052 | H | H | H | —N(OH)C(O)Me | 0 | — |
| 1.053 | H | H | H | —ONHC(O)Me | 0 | — |

TABLE 2
This table discloses 49 specific compounds of the formula (T-2):
(T-2)

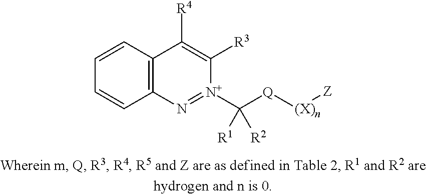

| Compound | | | | | | |
|---|---|---|---|---|---|---|
| number | R³ | R⁴ | R⁵ | Z | m | Q |
| 2.001 | H | H | H | —C(O)OH | 1 | CH₂ |
| 2.002 | H | H | H | —C(O)OMe | 1 | CH₂ |
| 2.003 | H | H | H | —C(O)NHOMe | 1 | CH₂ |
| 2.004 | H | H | H | —OC(O)NHOMe | 1 | CH₂ |
| 2.005 | H | H | H | —NHC(O)NHOMe | 1 | CH₂ |
| 2.006 | H | H | H | —NMeC(O)NHOMe | 1 | CH₂ |
| 2.007 | H | H | H | —C(O)NHS(O)₂Me | 1 | CH₂ |
| 2.008 | H | H | H | —OC(O)NHS(O)₂Me | 1 | CH₂ |
| 2.009 | H | H | H | —NHC(O)NHS(O)₂Me | 1 | CH₂ |
| 2.010 | H | H | H | —NMeC(O)NHS(O)₂Me | 1 | CH₂ |
| 2.011 | H | H | H | —S(O)₂OH | 1 | CH₂ |
| 2.012 | H | H | H | —OS(O)₂OH | 1 | CH₂ |
| 2.013 | H | H | H | —NHS(O)₂OH | 1 | CH₂ |
| 2.014 | H | H | H | —NMeS(O)₂OH | 1 | CH₂ |
| 2.015 | H | H | H | —S(O)OH | 1 | CH₂ |
| 2.016 | H | H | H | —OS(O)OH | 1 | CH₂ |
| 2.017 | H | H | H | —NHS(O)OH | 1 | CH₂ |
| 2.018 | H | H | H | —NMeS(O)OH | 1 | CH₂ |
| 2.019 | H | H | H | —NHS(O)₂CF₃ | 1 | CH₂ |
| 2.020 | H | H | H | —S(O)₂NHC(O)Me | 1 | CH₂ |
| 2.021 | H | H | H | —OS(O)₂NHC(O)Me | 1 | CH₂ |
| 2.022 | H | H | H | —NHS(O)₂NHC(O)Me | 1 | CH₂ |
| 2.023 | H | H | H | —NMeS(O)₂NHC(O)Me | 1 | CH₂ |
| 2.024 | H | H | H | —P(O)(OH)(OMe) | 1 | CH₂ |
| 2.025 | H | H | H | —P(O)(OH)(OH) | 1 | CH₂ |
| 2.026 | H | H | H | —OP(O)(OH)(OMe) | 1 | CH₂ |
| 2.027 | H | H | H | —OP(O)(OH)(OH) | 1 | CH₂ |
| 2.028 | H | H | H | —NHP(O)(OH)(OMe) | 1 | CH₂ |
| 2.029 | H | H | H | —NHP(O)(OH)(OH) | 1 | CH₂ |
| 2.030 | H | H | H | —NMeP(O)(OH)(OMe) | 1 | CH₂ |
| 2.031 | H | H | H | —NMeP(O)(OH)(OH) | 1 | CH₂ |
| 2.032 | H | H | H | -tetrazole | 1 | CH₂ |
| 2.033 | H | H | H | —S(O)₂OH | 2 | CH₂CH(NH₂) |
| 2.034 | H | H | H | —C(O)OH | 2 | CH₂CH(NH₂) |
| 2.035 | H | H | H | —C(O)NHCN | 1 | CH₂ |
| 2.036 | H | H | H | —OC(O)NHCN | 1 | CH₂ |
| 2.037 | H | H | H | —NHC(O)NHCN | 1 | CH₂ |
| 2.038 | H | H | H | —NMeC(O)NHCN | 1 | CH₂ |
| 2.039 | H | H | H | —S(O)₂NHCN | 1 | CH₂ |
| 2.040 | H | H | H | —OS(O)₂NHCN | 1 | CH₂ |
| 2.041 | H | H | H | —NHS(O)₂NHCN | 1 | CH₂ |
| 2.042 | H | H | H | —NMeS(O)₂NHCN | 1 | CH₂ |
| 2.043 | H | H | H | —S(O)₂NHS(O)₂Me | 1 | CH₂ |
| 2.044 | H | H | H | —OS(O)₂NHS(O)₂Me | 1 | CH₂ |
| 2.045 | H | H | H | —NHS(O)₂NHS(O)₂Me | 1 | CH₂ |
| 2.046 | H | H | H | —NMeS(O)₂NHS(O)₂Me | 1 | CH₂ |
| 2.047 | H | H | H | —P(O)H(OH) | 1 | CH₂ |
| 2.048 | H | H | H | —N(OH)C(O)Me | 1 | CH₂ |
| 2.049 | H | H | H | —ONHC(O)Me | 1 | CH₂ |

TABLE 3
This table discloses 49 specific compounds of the formula (T-3):
(T-3)

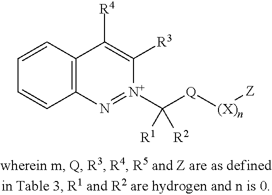

| Com- | | | | | | |
|---|---|---|---|---|---|---|
| pound | | | | | | |
| number | R³ | R⁴ | R⁵ | Z | m | Q |
| 3.001 | H | H | H | —C(O)OH | 2 | CH₂CH₂ |
| 3.002 | H | H | H | —C(O)OMe | 2 | CH₂CH₂ |
| 3.003 | H | H | H | —C(O)NHOMe | 2 | CH₂CH₂ |
| 3.004 | H | H | H | —OC(O)NHOMe | 2 | CH₂CH₂ |
| 3.005 | H | H | H | —NHC(O)NHOMe | 2 | CH₂CH₂ |
| 3.006 | H | H | H | —NMeC(O)NHOMe | 2 | CH₂CH₂ |
| 3.007 | H | H | H | —C(O)NHS(O)₂Me | 2 | CH₂CH₂ |
| 3.008 | H | H | H | —OC(O)NHS(O)₂Me | 2 | CH₂CH₂ |
| 3.009 | H | H | H | —NHC(O)NHS(O)₂Me | 2 | CH₂CH₂ |
| 3.010 | H | H | H | —NMeC(O)NHS(O)₂Me | 2 | CH₂CH₂ |
| 3.011 | H | H | H | —S(O)₂OH | 2 | CH₂CH₂ |
| 3.012 | H | H | H | —OS(O)₂OH | 2 | CH₂CH₂ |
| 3.013 | H | H | H | —NHS(O)₂OH | 2 | CH₂CH₂ |
| 3.014 | H | H | H | —NMeS(O)₂OH | 2 | CH₂CH₂ |
| 3.015 | H | H | H | —S(O)OH | 2 | CH₂CH₂ |
| 3.016 | H | H | H | —OS(O)OH | 2 | CH₂CH₂ |
| 3.017 | H | H | H | —NHS(O)OH | 2 | CH₂CH₂ |
| 3.018 | H | H | H | —NMeS(O)OH | 2 | CH₂CH₂ |
| 3.019 | H | H | H | —NHS(O)₂CF₃ | 2 | CH₂CH₂ |
| 3.020 | H | H | H | —S(O)₂NHC(O)Me | 2 | CH₂CH₂ |
| 3.021 | H | H | H | —OS(O)₂NHC(O)Me | 2 | CH₂CH₂ |
| 3.022 | H | H | H | —NHS(O)₂NHC(O)Me | 2 | CH₂CH₂ |
| 3.023 | H | H | H | —NMeS(O)₂NHC(O)Me | 2 | CH₂CH₂ |
| 3.024 | H | H | H | —P(O)(OH)(OMe) | 2 | CH₂CH₂ |
| 3.025 | H | H | H | —P(O)(OH)(OH) | 2 | CH₂CH₂ |
| 3.026 | H | H | H | —OP(O)(OH)(OMe) | 2 | CH₂CH₂ |
| 3.027 | H | H | H | —OP(O)(OH)(OH) | 2 | CH₂CH₂ |
| 3.028 | H | H | H | —NHP(O)(OH)(OMe) | 2 | CH₂CH₂ |
| 3.029 | H | H | H | —NHP(O)(OH)(OH) | 2 | CH₂CH₂ |
| 3.030 | H | H | H | —NMeP(O)(OH)(OMe) | 2 | CH₂CH₂ |
| 3.031 | H | H | H | —NMeP(O)(OH)(OH) | 2 | CH₂CH₂ |
| 3.032 | H | H | H | -tetrazole | 2 | CH₂CH₂ |
| 3.033 | H | H | H | —S(O)₂OH | 3 | CH₂CH₂CH(NH₂) |
| 3.034 | H | H | H | —C(O)OH | 3 | CH₂CH₂CH(NH₂) |
| 3.035 | H | H | H | —C(O)NHCN | 2 | CH₂CH₂ |
| 3.036 | H | H | H | —OC(O)NHCN | 2 | CH₂CH₂ |
| 3.037 | H | H | H | —NHC(O)NHCN | 2 | CH₂CH₂ |
| 3.038 | H | H | H | —NMeC(O)NHCN | 2 | CH₂CH₂ |
| 3.039 | H | H | H | —S(O)₂NHCN | 2 | CH₂CH₂ |
| 3.040 | H | H | H | —OS(O)₂NHCN | 2 | CH₂CH₂ |
| 3.041 | H | H | H | —NHS(O)₂NHCN | 2 | CH₂CH₂ |
| 3.042 | H | H | H | —NMeS(O)₂NHCN | 2 | CH₂CH₂ |
| 3.043 | H | H | H | —S(O)₂NHS(O)₂Me | 2 | CH₂CH₂ |
| 3.044 | H | H | H | —OS(O)₂NHS(O)₂Me | 2 | CH₂CH₂ |
| 3.045 | H | H | H | —NHS(O)₂NHS(O)₂Me | 2 | CH₂CH₂ |
| 3.046 | H | H | H | —NMeS(O)₂NHS(O)₂Me | 2 | CH₂CH₂ |
| 3.047 | H | H | H | —P(O)H(OH) | 2 | CH₂CH₂ |
| 3.048 | H | H | H | —N(OH)C(O)Me | 2 | CH₂CH₂ |
| 3.049 | H | H | H | —ONHC(O)Me | 2 | CH₂CH₂ |

TABLE 4
This table discloses 17 specific compounds of the formula (T-4):
(T-4)

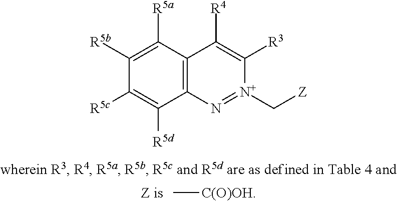

| Compound | | | | | | |
|---|---|---|---|---|---|---|
| number | R³ | R⁴ | R^{5a} | R^{5b} | R⁵ | R^{5d} |
| 1.001 | —Me | H | H | H | H | H |
| 1.002 | H | —Me | H | H | H | H |
| 1.003 | H | —OMe | H | H | H | H |
| 1.004 | H | —CONHMe | H | H | H | H |
| 1.005 | H | —CONMe₂ | H | H | H | H |
| 1.006 | H | H | —Me | H | H | H |
| 1.007 | H | H | —OMe | H | H | H |
| 1.008 | H | H | —Cl | H | H | H |
| 1.009 | H | H | —F | H | H | H |
| 1.010 | H | H | H | H | —Me | H |
| 1.011 | H | H | H | H | —OMe | H |
| 1.012 | H | H | H | H | —Cl | H |
| 1.013 | H | H | H | H | —F | H |
| 1.014 | H | H | H | H | H | —Me |
| 1.015 | H | H | H | H | H | —OMe |
| 1.016 | H | H | H | H | H | —CONHMe |
| 1.017 | H | H | H | H | H | —CONMe₂ |

TABLE 5
This table discloses 17 specific compounds of the formula (T-5):

| | |
|---|---|
| 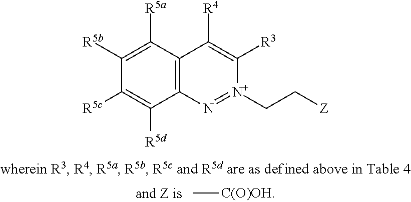 | (T-5) |

TABLE 6
This table discloses 17 specific compounds of the formula (T-6):

| | |
|---|---|
| 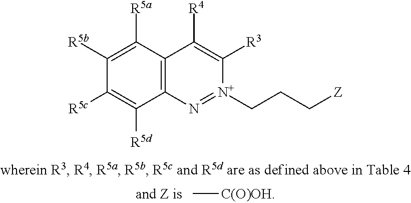 | (T-6) |

TABLE 7
This table discloses 17 specific compounds of the formula (T-7):

| | |
|---|---|
| 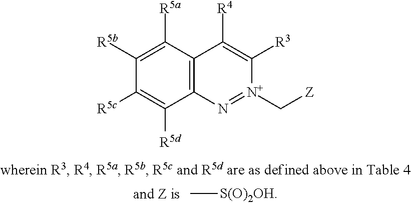 | (T-7) |

TABLE 8
This table discloses 17 specific compounds of the formula (T-8):

| | |
|---|---|
| 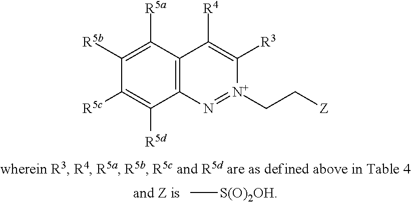 | (T-8) |

TABLE 9
This table discloses 17 specific compounds of the formula (T-9):

| | |
|---|---|
| 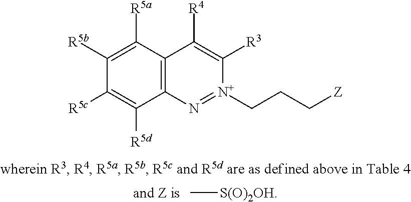 | (T-9) |

TABLE 10
This table discloses 17 specific compounds of the formula (T-10):

| | |
|---|---|
| 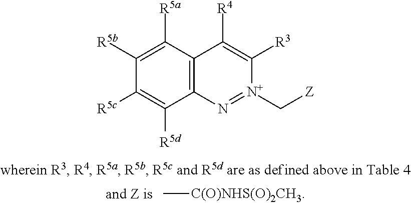 | (T-10) |

TABLE 11
This table discloses 17 specific compounds of the formula (T-11):

| | |
|---|---|
| 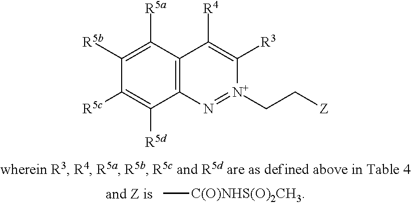 | (T-11) |

TABLE 12
This table discloses 17 specific compounds of the formula (T-12):

| | |
|---|---|
| 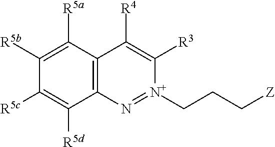 | (T-12) |
| wherein R³, R⁴, R^{5a}, R^{5b}, R^{5c} and R^{5d} are as defined | |
| above in Table 4 and Z is —C(O)NHS(O)₂CH₃. | |

TABLE 13
This table discloses 17 specific compounds of the formula (T-13):

| | |
|---|---|
| 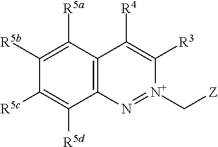 | (T-13) |
| wherein R³, R⁴, R^{5a}, R^{5b}, R^{5c} and R^{5d} are as defined | |
| above in Table 4 and Z is —P(O)(OH)(OCH₃). | |

TABLE 14
This table discloses 17 specific compounds of the formula (T-14):

| | |
|---|---|
| 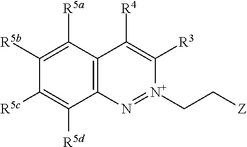 | (T-14) |
| wherein R³, R⁴, R^{5a}, R^{5b}, R^{5c} and R^{5d} are as defined | |
| above in Table 4 and Z is —P(O)(OH)(OCH₃). | |

TABLE 15
This table discloses 17 specific compounds of the formula (T-15):

| | |
|---|---|
| 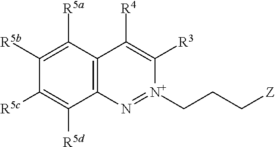 | (T-15) |
| wherein R³, R⁴, R^{5a}, R^{5b}, R^{5c} and R^{5d} are as defined | |
| above in Table 4 and Z is —P(O)(OH)(OCH₃). | |

TABLE 16
This table discloses 17 specific compounds of the formula (T-16):

| | |
|---|---|
| 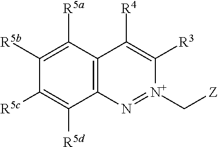 | (T-16) |
| wherein R³, R⁴, R^{5a}, R^{5b}, R^{5c} and R^{5d} are as | |
| defined above in Table 4 and Z is —P(O)(OH)(OH). | |

TABLE 17
This table discloses 17 specific compounds of the formula (T-17):

| | |
|---|---|
| 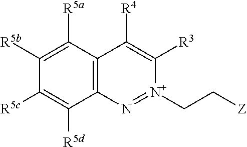 | (T-17) |
| wherein R³, R⁴, R^{5a}, R^{5b}, R^{5c} and R^{5d} are as | |
| defined above in Table 4 and Z is —P(O)(OH)(OH). | |

TABLE 18
This table discloses 17 specific compounds of the formula (T-18):

| | |
|---|---|
| 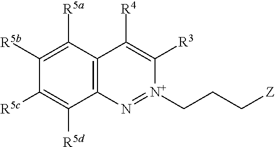 | (T-18) |
| wherein R³, R⁴, R^{5a}, R^{5b}, R^{5c} and R^{5d} are as | |
| defined above in Table 4 and Z is —P(O)(OH)(OH). | |

TABLE 19
This table discloses 19 specific compounds of the formula (T-19):

| | |
|---|---|
| 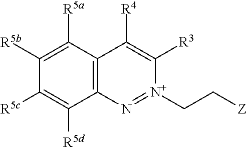 | (T-19) |
| wherein R³, R⁴, R^{5a}, R^{5b}, R^{5c} and R^{5d} are as | |
| defined above in Table 4 and Z is —OS(O)₂OH. | |

TABLE 20
This table discloses 17 specific compounds of the formula (T-20):

| | |
|---|---|
| 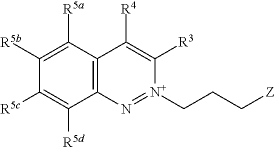 | (T-20) |
| wherein R³, R⁴, R^{5a}, R^{5b}, R^{5c} and R^{5d} are as defined | |
| above in Table 4 and Z is —OS(O)₂OH. | |

TABLE 21
This table discloses 17 specific compounds of the formula (T-21):

| | |
|---|---|
| 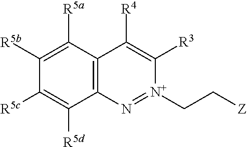 | (T-21) |
| wherein R³, R⁴, R^{5a}, R^{5b}, R^{5c} and R^{5d} are as | |
| defined above in Table 4 and Z is —NHS(O)₂OH. | |

TABLE 22
This table discloses 17 specific compounds of the formula (T-22):

| | |
|---|---|
| 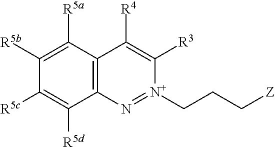 | (T-22) |
| wherein R³, R⁴, R^{5a}, R^{5b}, R^{5c} and R^{5d} are as | |
| defined above in Table 4 and Z is —NHS(O)₂OH. | |

The compounds of the present invention may be prepared according to the following schemes in which the substituents k, n, m, r, Q, X, Z, R¹, R², R^{1a}, R^{2b}, R², R³, R⁴, R⁵, R⁶, R⁷, R^{7a}, R^{7b}, R^{7c}, R⁸, R⁹, R¹⁰, R¹¹, R¹², R¹³, R¹⁴, R¹⁵, R^{15a}, R¹⁶, R¹⁷ and R¹⁸ are as defined hereinbefore unless explicitly stated otherwise. The compounds of the preceeding Tables 1 to 22 may thus be obtained in an analogous manner.

The compounds of formula (I) may be prepared by the alkylation of compounds of formula (X), wherein R³, R⁴, R⁵ and k are as defined for compounds of formula (I), with a suitable alkylating agent of formula (W), wherein R¹, R², Q, X and Z are as defined for compounds of formula (I) and LG is a suitable leaving group, for example, halide or pseudohalide such as triflate, mesylate ortosylate, in a suitable solvent at a suitable temperature, as described in reaction scheme 1. Example conditions include stirring a compound of formula (X) with an alkylating agent of formula (W) in a solvent, or mixture of solvents, such as acetone, dichloromethane, dichloroethane, N,N-dimethylformamide, acetonitrile, 1,4-dioxane, water, acetic acid or trifluroacetic acid at a temperature between −78° C. and 150° C. An alkylating agent of formula (W) may include, but is not limited to, bromoacetic acid, methyl bromoacetate, 3-bromopropionoic acid, methyl 3-bromopropionate, 2-bromo-N-methoxyacetamide, sodium 2-bromoethanesulphonate, 2,2-dimethylpropyl 2-(trifluoromethylsulfonyloxy)ethanesulfonate, 2-bromo-N-methanesulfonylacetamide, 3-bromo-N-methanesulfonylpropanamide, dimethoxyphosphorylmethyl trifluoromethanesulfonate, dimethyl 3-bromopropylphosphonate, 3-chloro-2,2-dimethyl-propanoic acid and diethyl 2-bromoethylphosphonate. Such alkylating agents and related compounds are either known in the literature or may be prepared by known literature methods. Compounds of formula (I) which may be described as esters of N-alkyl acids, which include, but are not limited to, esters of carboxylic acids, phosphonic acids, phosphinic acids, sulfonic acids and sulfinic acids, may be subsequently partially or fully hydrolysed by treatment with a suitable reagent, for example, aqueous hydrochloric acid or trimethylsilyl bromide, in a suitable solvent at a suitable temperature between 0° C. and 100° C.

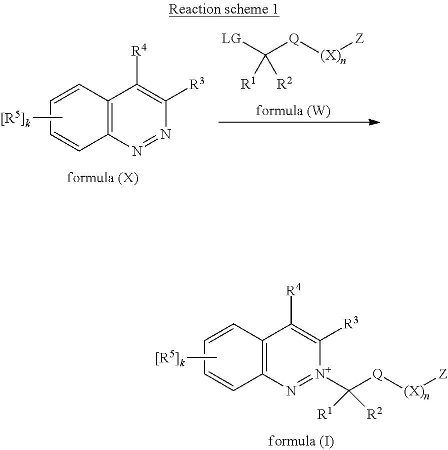

Additionally, compounds of formula (I) may be prepared by reacting compounds of formula (X), wherein R³, R⁴, R⁵ and k are as previously defined, with a suitably activated electrophilic alkene of formula (B), wherein R¹, R² and R^{1a} are as defined for compounds of formula (I) and Z is —S(O)₂OR¹⁰, —P(O)(R¹³)(OR¹⁰) or —C(O)OR¹⁰, in a suitable solvent at a suitable temperature. Compounds of formula (B) are known in the literature, or may be prepared by known methods. Example reagents include, but are not limited to, acrylic acid, methacrylic acid, crotonic acid, 3,3-dimethylacrylic acid, methyl acrylate, ethene sulfonic acid, isopropyl ethylenesulfonate, 2,2-dimethylpropyl ethenesulfonate and dimethyl vinylphosphonate. The direct products of these reactions, which may be described as esters of N-alkyl acids, which include, but are not limited to, esters of carboxylic acids, phosphonic acids, phosphinic acids, sulfonic acids and sulfinic acids, may be subsequently partially or fully hydrolysed by treatment with a suitable reagent in a suitable solvent at a suitable temperature, as described in reaction scheme 2.

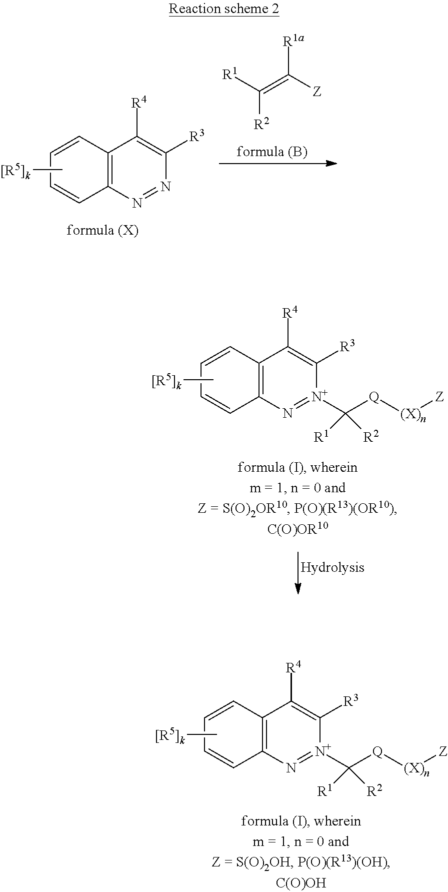

In a related reaction compounds of formula (I), wherein Q is C(R^{1a}R^{2b}), m is 1, 2 or 3, n=0 and Z is —S(O)₂OH, —OS(O)₂OH or —NR⁶S(O)₂OH, may be prepared by the reaction of compounds of formula (X), wherein R³, R⁴, R⁵ and k are as previously defined, with a cyclic alkylating agent of formula (E), (F) or (AF), wherein Y is C(R^{1a}R^{2b}), O or NR⁶ and R¹, R², R^{1a} and R^{2b} are as defined for compounds of formula (I), in a suitable solvent at a suitable temperature, as described in reaction scheme 3. Suitable solvents and suitable temperatures are as previously described. An alkylating agent of formula (E) or (F) may include, but is not limited to, 1,3-propanesultone, 1,4-butanesultone, ethylenesulfate, 1,3-propylene sulfate and 1,2,3-oxathiazolidine 2,2-dioxide. Such alkylating agents and related compounds are either known in the literature or may be prepared by known literature methods.

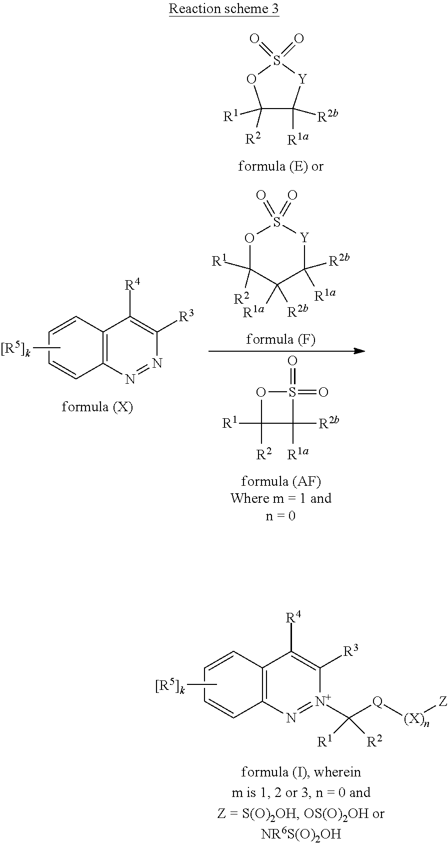

A compound of formula (I), wherein m is 0, n is 0 and Z is S(O)₂OH, may be prepared from a compound of formula (I), wherein m is 0, n is 0 and Z is C(O)OR¹⁰, by treatment with trimethylsilylchlorosulfonate in a suitable solvent at a suitable temperature, as described in reaction scheme 4. Preferred conditions include heating the carboxylate precursor in neat trimethylsilylchlorosulfonate at a temperature between 25° C. and 150° C.

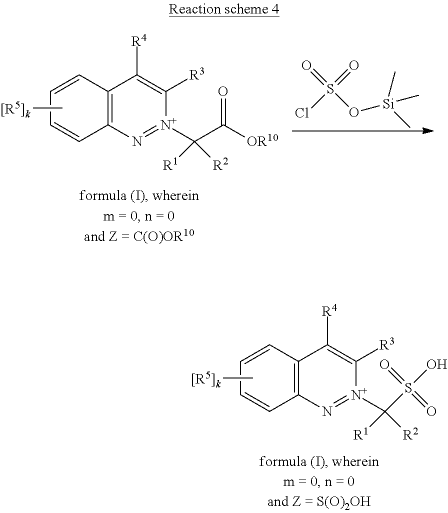

Furthermore, compounds of formula (I) may be prepared by reacting compounds of formula (X), wherein R³, R⁴, R⁵ and k are as previously defined, with a suitable alcohol of formula (WW), wherein n, R¹, R², Q, X and Z are as defined for compounds of formula (I), under Mitsunobu-type conditions such as those reported by Petit et al, Tet. Lett. 2008, 49 (22), 3663. Suitable phosphines include triphenylphosphine, suitable azodicarboxylates include diisopropylazodicarboxylate and suitable acids include fluoroboric acid, triflic acid and bis(trifluoromethylsulfonyl)amine, as described in reaction scheme 5. Such alcohols are either known in the literature or may be prepared by known literature methods.

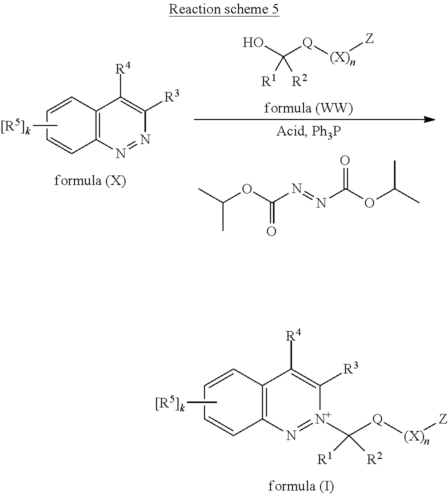

The synthesis of compounds of formula (X) may be achieved following procedures including, but not limited to, classical von Richter (for example Von Richter, V. Chem. Ber., 1883, 677-683), Borsche-Koelsch (for example Borsche, W.; Herbert, A. Liebigs Ann. Chem., 1941, 546, 293, and Koelsch, C. F. J. Org. Chem., 1943, 8, 295), Neber-Bossel (for example Baumgarten, H. E.; Creger, P. L. J. Am. Chem. Soc., 1960, 82 (17), 4634-4638) and Widman-Stoermer (for example Widman, O. Chem. Ber., 1884, 17, 722, and Stoermer, R.; Fincke, H. Chem. Ber., 1909, 42, 3115) cinnoline syntheses.

In one approach a compound of formula (X), wherein R⁴ is hydrogen and R³, R⁵ and k are as previously defined, may be prepared by a sequence starting with the diazotisation of an optionally substituted 2-alkynylaniline of formula (G), as described in reaction scheme 6, with either an inorganic nitrite or alkyl nitrite in the presence of acid in a suitable solvent at a suitable temperature (for example Von Richter, V. Chem. Ber., 1883, 677-683) to afford a cinnoline of formula (H) or of formula (J). A compound of formula (H) may be converted to a compound of formula (J), wherein Hal is chlorine or bromine, by treatment with known halogenating agents, such as a phosphoryl halide, in a suitable solvent at a suitable temperature (for example Ruchelman, A. L. et al Bioorg. Med. Chem., 2004, 12(4), 795-806). A compound of formula (J), wherein Hal is chlorine or bromine, may be reduced to a compound of formula (X), wherein R⁴ is hydrogen, by a variety of methods including treatment with tosyl hydrazine, to give a compound of formula (P), followed by base, such as aqueous sodium carbonate, in a suitable solvent at a suitable temperature (for example Osborn, A. R.; Schofield, K. J. Chem. Soc., 1956, 4207-13). Compounds of formula (G) are known in the literature or may be prepared by known methods (for example Moody, D. L. et al Bioorg. Med. Chem. Lett., 2007, 17(8), 2380-2384).

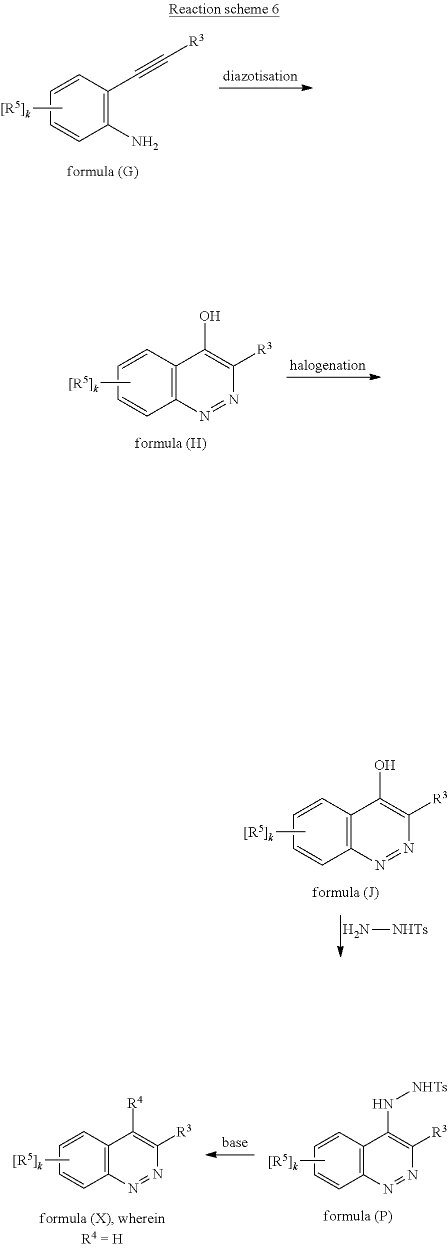

A compound of formula (X), wherein both R³ and R⁴ are hydrogen, may be prepared by an analogous method starting from a compound of formula (G) wherein R³ is SiMe₃ or CO₂H. The direct products of the cyclisation may either deprotect under the conditions of the reaction as in the case where R³ is SiMe₃ (for example Chapoulaud V. G. et al Tetrahedron, 2000, 56, 5499), or may require a subsequent deprotection step as in the case where R³ is CO₂H (for example Schofield, K.; Simpson, J. C. E. J. Chem. Soc., 1945, 512-520).

In a related reaction cinnolines of formula (X), wherein both R³ and R⁴ are hydrogen, may be prepared by the thermal rearrangement of compounds of formula (K) under neutral conditions. Triazenes of formula (K) may be prepared by the diazotization of 2-ethynylanilines of formula (G), wherein R³ is hydrogen, followed by trapping with an amine such as diethylamine (for example Kehoe, J. M. et al Org. Lett., 2000, 2(7), 969-972). These triazenes may be heated in an appropriate solvent at an appropriate temperature, such as dichlorobenzene at 200° C., to achieve the desired cyclisation (for example Kimball, D. B. et al J. Org. Chem., 2002, 67(18), 6395-6405), as described in reaction scheme 7.

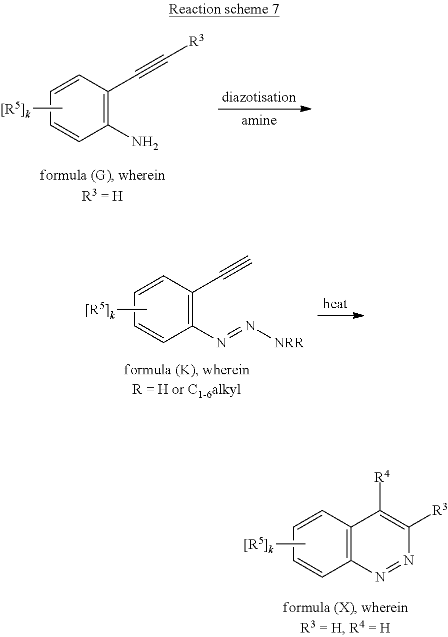

In another approach a compound of formula (X), wherein R⁴ is hydrogen, may be prepared by a sequence starting with the diazotisation of an optionally substituted 2-aminoarylketone of formula (L) with either an inorganic nitrite or alkyl nitrite in the presence of acid in a suitable solvent at a suitable temperature (for example Borsche, W.; Herbert, A. Liebigs Ann. Chem., 1941, 546, 293, and Koelsch, C. F. J. Org. Chem., 1943, 8, 295) to afford a cinnoline of formula (H), as described in reaction scheme 8. A compound of formula (H) may be further derivatised as described previously. Compounds of formula (L) are known in the literature or may be prepared by known methods (for example Jana, S. et al Org. Biomol. Chem., 2015, 13(31), 8411-8415).

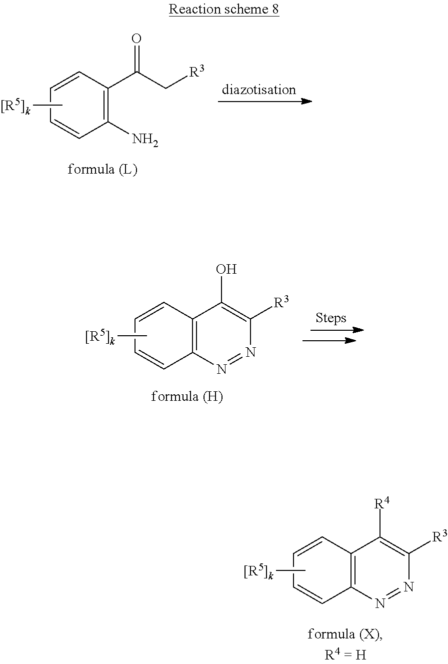

wherein In a further approach a compound of formula (X), wherein R³ is halogen and R⁴ is hydrogen, may be prepared by a sequence, as described in reaction scheme 9, starting with the diazotisation of an optionally substituted 2-aminomandelic acid of formula (M) with either an inorganic nitrite or alkyl nitrite in the presence of acid in a suitable solvent at a suitable temperature (for example Baumgarten, H. E.; Creger, P. L. J. Am. Chem. Soc., 1960, 82 (17), 4634-4638). The derived diazo compound of formula (N) may be reduced to the corresponding 2-hydrazinomandelic acid of formula (O) by treatment with an appropriate reducing agent, such as tin chloride in aqueous hydrochloric acid, in an appropriate solvent at an appropriate temperature (for example Alford, E. J.; Schofield, K. J. Chem. Soc., 1953, 2102-2108). These intermediates may be cyclized to the corresponding 3-hydroxycinnolines of formula (PP) under acidic conditions in an appropriate solvent at an appropriate temperature, such as boiling aqueous hydrochloric acid (for example Alford, E. J.; Schofield, K. J. Chem. Soc., 1952, 2102-2108). A cinnoline of formula (PP) may be further converted to a compound of formula (X), wherein R³ is halogen and R⁴ is hydrogen, by halogenation under conditions analogous to those described in reaction scheme 6. Compounds of formula (M) are known in the literature or may be prepared by known methods (for example Alford, E. J.; Schofield, K. J. Chem. Soc., 1952, 2102-2108).

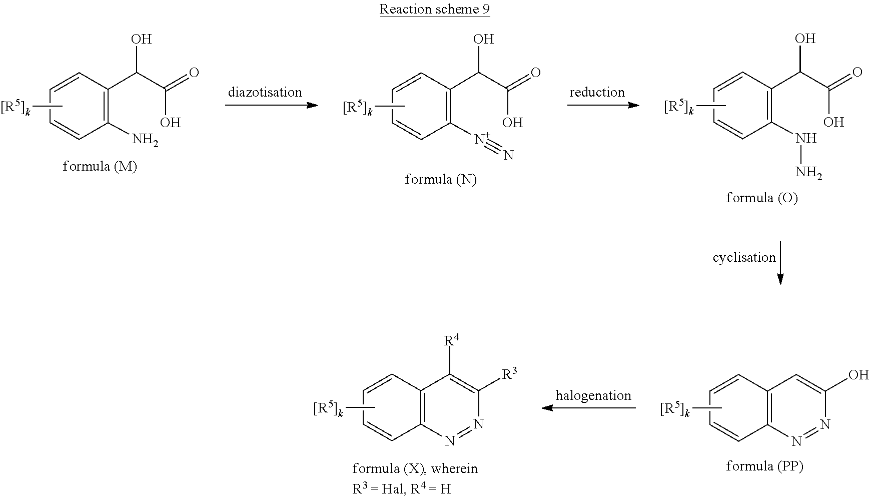

In a further approach a compound of formula (X) may be prepared by the diazotisation of a 2-aminostyrene of formula (Q) with either an inorganic nitrite or alkyl nitrite in the presence of acid in a suitable solvent at a suitable temperature (for example, Widman, O. Chem. Ber., 1884, 17, 722, and Stoermer, R.; Fincke, H. Chem. Ber., 1909, 42, 3115), as described in reaction scheme 10. Compounds of formula (Q) are known in the literature or may be prepared by known methods (for example, Kobayashi, K. et al Heterocycles, 2008, 75(1), 95-105).

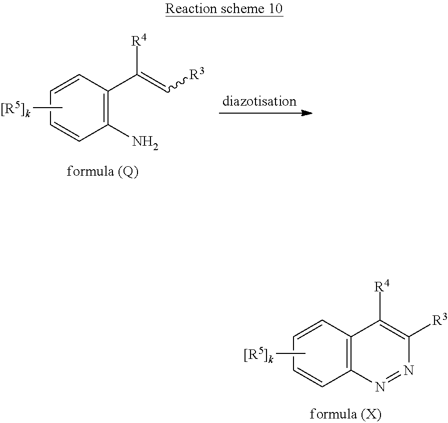

In a further approach a compound of formula (X) may be prepared by a sequence starting with the oxidation of a compound of formula (R), wherein Hal is a halogen or pseudo-halogen such as mesylate, tosylate or triflate, using a suitable oxidizing agent in a suitable solvent at a suitable temperature, for example selenium dioxide in 1,4-dioxane at a temperature between 25° C. to 100° C., as described in reaction scheme 11. Compounds of formula (S) may be condensed with an optionally protected hydrazine, wherein PG is a protecting group, such as tert-butyl carbazate, to afford a hydrazone of formula (T), preferably in the presence of an acid catalyst in a suitable solvent at a suitable temperature. Cyclisation may be achieved by treatment with a suitable base in a suitable solvent at a suitable temperature, for example potassium carbonate in N,N-dimethylformamide at a temperature between 25° C. and 150° C. Compounds of formula (H) may be further derivatised as described previously. Compounds of formula (R) are known in the literature or may be prepared by known methods (for example, Ruan, J. et al J. Am. Chem. Soc., 132(46), 16689-16699; 2010 and Ridge, D. N. et al J. Med. Chem., 1979, 22(11), 1385-1389).

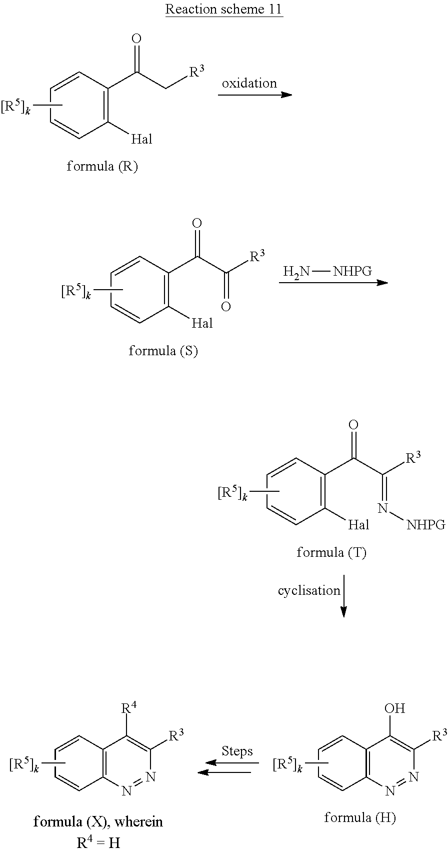

A compound of formula (T) and a compound of formula (J), wherein Hal is a halogen or pseudo-halogen such as mesylate, tosylate or triflate, may both be derivatised by a range of transition-metal catalyzed cross couplings, including but not limited to, Suzuki (for example Heiter, H. J. et al J. Heterocyclic. Chem., 2013, 50(1), 141-144), Negishi (for example see WO2015/086523), Stille (for example Bui, C. T.; Flynn, B. L. Mol. Divers., 2011, 15(1), 83-89) Sonogashira (for example Heiter, H. J. et al J. Heterocyclic. Chem., 2013, 50(1), 141-144) and Heck (for example Ames, D. E.; Bull, D. Tetrahedron, 1982, 38, 383), as described in reaction scheme 12. Transition metal catalysts, ligands, bases, solvents and temperatures may be selected with reference to the desired coupling and are known in the literature.

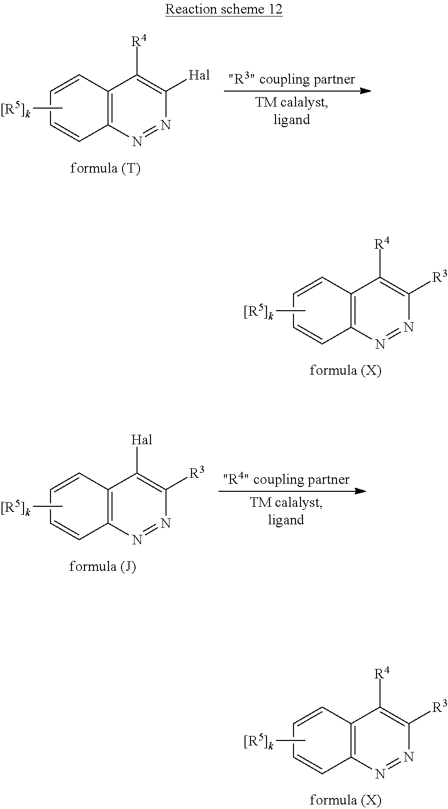

A compound of formula (T) and a compound of formula (J), as previously described, may both be further derivatised by substitution with various nucleophiles to afford a compound of formula (X). Suitable nucleophiles include, but are not limited to, optionally substituted alcohols, amines, thiols and sulfinates. Such a substitution is preferably achieved at the C4 position, and these reactions are known in the literature.

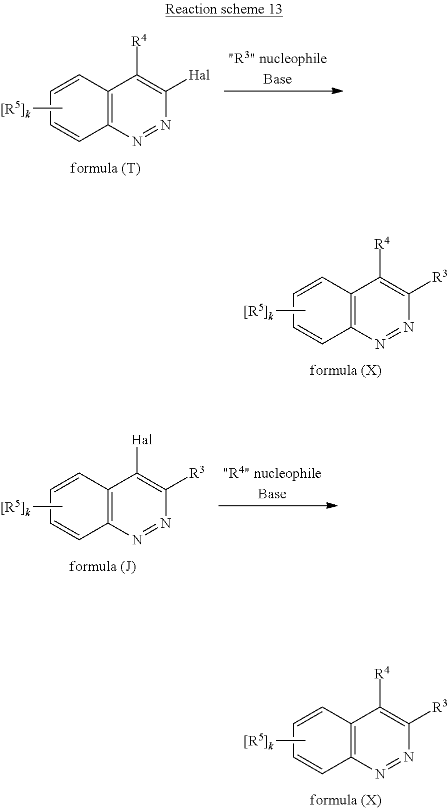

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of Formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The composition of the present may further comprise at least one additional pesticide. For example, the compounds according to the invention can also be used in combination with other herbicides or plant growth regulators. In a preferred embodiment the additional pesticide is a herbicide and/or herbicide safener.

Thus, compounds of formula (I) can be used in combination with one or more other herbicides to provide various herbicidal mixtures. Specific examples of such mixtures include (wherein “I” represents a compound of formula (I)): —I+acetochlor, I+acifluorfen (including acifluorfen-sodium), I+aclonifen, I+ametryn, I+amicarbazone, I+aminopyralid, I+aminotriazole, I+atrazine, I+beflubutamid-M, I+bensulfuron (including bensulfuron-methyl), I+bentazone, I+bicyclopyrone, I+bilanafos, I+bispyribac-sodium, I+bixlozone, I+bromacil, I+bromoxynil, I+butachlor, I+butafenacil, I+carfentrazone (including carfentrazone-ethyl), I+cloransulam (including cloransulam-methyl), I+chlorimuron (including chlorimuron-ethyl), I+chlorotoluron, I+chlorsulfuron, I+cinmethylin, I+clacyfos, I+clethodim, I+clodinafop (including clodinafop-propargyl), I+clomazone, I+clopyralid, I+cyclopyranil, I+cyclopyrimorate, I+cyclosulfamuron, I+cyhalofop (including cyhalofop-butyl), I+2,4-D (including the choline salt and 2-ethylhexyl ester thereof), I+2,4-DB, I+desmedipham, I+dicamba (including the aluminium, aminopropyl, bis-aminopropylmethyl, choline, dichloroprop, diglycolamine, dimethylamine, dimethylammonium, potassium and sodium salts thereof) I+diclosulam, I+diflufenican, I+diflufenzopyr, I+dimethachlor, I+dimethenamid-P, I+diquat dibromide, diuron, I+ethalfluralin, I+ethofumesate, I+fenoxaprop (including fenoxaprop-P-ethyl), I+fenoxasulfone, I+fenquinotrione, I+fentrazamide, I+flazasulfuron, I+florasulam, I+florpyrauxifen (including florpyrauxifen-benzyl), I+fluazifop (including fluazifop-P-butyl), I+flucarbazone (including flucarbazone-sodium), I+flufenacet, I+flumetsulam, I+flumioxazin, I+fluometuron, I+flupyrsulfuron (including flupyrsulfuron-methyl-sodium), I+fluroxypyr (including fluroxypyr-meptyl), I+fomesafen, I+foramsulfuron, I+glufosinate (including the ammonium salt thereof), I+glyphosate (including the diammonium, isopropylammonium and potassium salts thereof), I+halauxifen (including halauxifen-methyl), I+haloxyfop (including haloxyfop-methyl), I+hexazinone, I+hydantocidin, I+imazamox, I+imazapic, I+imazapyr, I+imazethapyr, I+indaziflam, I+iodosulfuron (including iodosulfuron-methyl-sodium), I+iofensulfuron (including iofensulfuron-sodium), I+ioxynil, I+isoproturon, I+isoxaflutole, I+lancotrione, I+MCPA, I+MCPB, I+mecoprop-P, I+mesosulfuron (including mesosulfuron-methyl), I+mesotrione, I+metamitron, I+metazachlor, I+methiozolin, I+metolachlor, I+metosulam, I+metribuzin, I+metsulfuron, I+napropamide, I+nicosulfuron, I+norflurazon, I+oxadiazon, I+oxasulfuron, I+oxyfluorfen, I+paraquat dichloride, I+pendimethalin, I+penoxsulam, I+phenmedipham, I+picloram, I+pinoxaden, I+pretilachlor, I+primisulfuron-methyl, I+prometryne, I+propanil, I+propaquizafop, I+propyrisulfuron, I+propyzamide, I+prosulfocarb, I+prosulfuron, I+pyraclonil, I+pyraflufen (including pyraflufen-ethyl), I+pyrasulfotole, I+pyridate, I+pyriftalid, I+pyrimisulfan, I+pyroxasulfone, I+pyroxsulam, I+quinclorac, I+quinmerac, I+quizalofop (including quizalofop-P-ethyl and quizalofop-P-tefuryl), I+rimsulfuron, I+saflufenacil, I+sethoxydim, I+simazine, I+S-metalochlor, I+sulfentrazone, I+sulfosulfuron, I+tebuthiuron, I+tefuryltrione, I+tembotrione, I+terbuthylazine, I+terbutryn, I+tetflupyrolimet, I+thiencarbazone, I+thifensulfuron, I+tiafenacil, I+tolpyralate, I+topramezone, I+tralkoxydim, I+triafamone, I+triallate, I+triasulfuron, I+tribenuron (including tribenuron-methyl), I+triclopyr, I+trifloxysulfuron (including trifloxysulfuron-sodium), I+trifludimoxazin, I+trifluralin, I+triflusulfuron, I+ethyl 2-[[3-[2-chloro-4-fluoro-5-[3-methyl-2,6-dioxo-4-(trifluoromethyl)pyrimidin-1-yl]phenoxy]-2-pyridyl]oxy]acetate, I+3-(2-chloro-4-fluoro-5-(3-methyl-2,6-dioxo-4-trifluoro methyl-3,6-dihydropyrimidin-1(2H)-yl)phenyl)-5-methyl-4,5-dihydroisoxazole-5-carboxylic acid ethyl ester, I+4-hydroxy-1-methoxy-5-methyl-3-[4-(trifluoromethyl)-2-pyridyl]imidazolidin-2-one, I+4-hydroxy-1,5-dimethyl-3-[4-(trifluoromethyl)-2-pyridyl]imidazolidin-2-one, I+5-ethoxy-4-hydroxy-1-methyl-3-[4-(trifluoromethyl)-2-pyridyl]imidazolidin-2-one, I+4-hydroxy-1-methyl-3-[4-(trifluoromethyl)-2-pyridyl]imidazolidin-2-one, I+4-hydroxy-1,5-dimethyl-3-[1-methyl-5-(trifluoromethyl)pyrazol-3-yl]imidazolidin-2-one, I+(4R)1-(5-tert-butylisoxazol-3-yl)-4-ethoxy-5-hydroxy-3-methyl-imidazolidin-2-one, I+3-[2-(3,4-dimethoxyphenyl)-6-methyl-3-oxo-pyridazine-4-carbonyl]bicyclo[3.2.1]octane-2,4-dione, I+2-[2-(3,4-dimethoxyphenyl)-6-methyl-3-oxo-pyridazine-4-carbonyl]-5-methyl-cyclohexane-1,3-dione, I+2-[2-(3,4-dimethoxyphenyl)-6-methyl-3-oxo-pyridazine-4-carbonyl]cyclohexane-1,3-dione, I+2-[2-(3,4-dimethoxyphenyl)-6-methyl-3-oxo-pyridazine-4-carbonyl]-5,5-dimethyl-cyclohexane-1,3-dione, I+6-[2-(3,4-dimethoxyphenyl)-6-methyl-3-oxo-pyridazine-4-carbonyl]-2,2,4,4-tetramethyl-cyclohexane-1,3,5-trione, I+2-[2-(3,4-dimethoxyphenyl)-6-methyl-3-oxo-pyridazine-4-carbonyl]-5-ethyl-cyclohexane-1,3-dione, I+2-[2-(3,4-dimethoxyphenyl)-6-methyl-3-oxo-pyridazine-4-carbonyl]-4,4,6,6-tetramethyl-cyclohexane-1,3-dione, I+2-[6-cyclopropyl-2-(3,4-dimethoxyphenyl)-3-oxo-pyridazine-4-carbonyl]-5-methyl-cyclohexane-1,3-dione, I+3-[6-cyclopropyl-2-(3,4-dimethoxyphenyl)-3-oxo-pyridazine-4-carbonyl]bicyclo[3.2.1]octane-2,4-dione, I+2-[6-cyclopropyl-2-(3,4-dimethoxyphenyl)-3-oxo-pyridazine-4-carbonyl]-5,5-dimethyl-cyclohexane-1,3-dione, I+6-[6-cyclopropyl-2-(3,4-dimethoxyphenyl)-3-oxo-pyridazine-4-carbonyl]-2,2,4,4-tetramethyl-cyclohexane-1,3,5-trione, I+2-[6-cyclopropyl-2-(3,4-dimethoxyphenyl)-3-oxo-pyridazine-4-carbonyl]cyclohexane-1,3-dione, I+4-[2-(3,4-dimethoxyphenyl)-6-methyl-3-oxo-pyridazine-4-carbonyl]-2,2,6,6-tetramethyl-tetrahydropyran-3,5-dione, I+4-[6-cyclopropyl-2-(3,4-dimethoxyphenyl)-3-oxo-pyridazine-4-carbonyl]-2,2,6,6-tetramethyl-tetrahydropyran-3,5-dione, I+4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate).

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Preferred combinations (wherein “I” represents a compound of formula (I)) include: —I+benoxacor, I+cloquintocet (including cloquintocet-mexyl); I+cyprosulfamide; I+dichlormid; I+fenchlorazole (including fenchlorazole-ethyl); I+fenclorim; I+fluxofenim; I+furilazole I+isoxadifen (including isoxadifen-ethyl); I+mefenpyr (including mefenpyr-diethyl); I+metcamifenand I+oxabetrinil.

Particularly preferred are mixtures of a compound of formula (I) with cyprosulfamide, isoxadifen (including isoxadifen-ethyl), cloquintocet (including cloquintocet-mexyl) and/or metcamifen.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

The compounds of formula (I) are also useful for pre-harvest desiccation in crops, for example, but not limited to, potatoes, soybean, sunflowers and cotton. Pre-harvest desiccation is used to desiccate crop foliage without significant damage to the crop itself to aid harvesting.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

| Suspension concentrate | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a | 1% |
| 75% emulsion in water) | |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### LIST OF ABBREVIATIONS

Boc=tert-butyloxycarbonyl
br=broad
CDCl₃=chloroform-d
CD₃OD=methanol-d
° C.=degrees Celsius
D₂O=water-d
DCM=dichloromethane
d=doublet
dd=double doublet
dt=double triplet
DMSO=dimethylsulfoxide
EtOAc=ethyl acetate
h=hour(s)
HCl=hydrochloric acid
HPLC=high-performance liquid chromatography (description of the apparatus and the methods used for HPLC are given below)
m=multiplet
M=molar
min=minutes
MHz=mega hertz
mL=millilitre
mp=melting point
ppm=parts per million
q=quartet
quin=quintet
rt=room temperature
s=singlet
t=triplet
THE=tetrahydrofuran

### LC/MS=Liquid Chromatography Mass Spectrometry

### Preparative Reverse Phase HPLC Method:

Compounds purified by mass directed preparative HPLC using ES+/ES− on a Waters FractionLynx Autopurification system comprising a 2767 injector/collector with a 2545 gradient pump, two 515 isocratic pumps, SFO, 2998 photodiode array (Wavelength range (nm): 210 to 400), 2424 ELSD and QDa mass spectrometer. A Waters Atlantis T3 5 micron 19×10 mm guard column was used with a Waters Atlantis T3 OBD, 5 micron 30×100 mm prep column.

### Ionisation Method:

Electrospray positive and negative: Cone (V) 20.00, Source Temperature (° C.) 120, Cone Gas Flow (L/Hr.) 50

Mass range (Da): positive 100 to 800, negative 115 to 800.

The preparative HPLC was conducted using an 11.4 minute run time (not using at column dilution, bypassed with the column selector), according to the following gradient table:

| Time (mins) | Solvent A (%) | Solvent B (%) | Flow (ml/min) |
|---|---|---|---|
| 0.00 | 100 | 0 | 35 |
| 2.00 | 100 | 0 | 35 |
| 2.01 | 100 | 0 | 35 |
| 7.0 | 90 | 10 | 35 |
| 7.3 | 0 | 100 | 35 |
| 9.2 | 0 | 100 | 35 |
| 9.8 | 99 | 1 | 35 |
| 11.35 | 99 | 1 | 35 |
| 11.40 | 99 | 1 | 35 |

515 pump 0 ml/min Acetonitrile (ACD)
515 pump 1 ml/min 90% Methanol/10% Water (make up pump)
Solvent A: Water with 0.05% Trifluoroacetic Acid
Solvent B: Acetonitrile with 0.05% Trifluoroacetic Acid

### Preparation Examples

### Example 1: Preparation of methyl 2-cinnolin-2-ium-2-ylacetate bromide A1

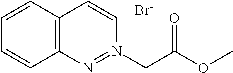

Cinnolin-2-ium chloride (0.2 g) was stirred in diethyl ether (6 mL) and 2M aqueous sodium hydroxide (3 mL) was added drop wise at room temperature. The reaction mixture was stirred for 30 minutes. The organic layer was concentrated and the residue was dissolved in acetone (6 mL). Methyl bromoacetate (0.176 mL) was added to the acetone solution and stirred for 22 hours at room temperature. The resulting precipitate was filtered off, washed with acetone and dried to afford methyl 2-cinnolin-2-ium-2-ylacetate bromide as a pale green solid.

¹H NMR (400 MHz, DMSO-d₆) 9.91 (d, 1H), 9.51 (d, 1H), 8.67 (d, 1H), 8.56 (d, 1H), 8.44 (br. s., 2H), 6.25 (br. s., 2H), 3.81 (s, 3H)

### Example 2: Preparation of 2-cinnolin-2-ium-2-ylacetate A2

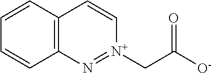 [SMILES: O=C([O-])C[n+]1ccc2ccccc2n1]

A mixture of methyl 2-cinnolin-2-ium-2-ylacetate bromide (0.2 g) and concentrated hydrochloric acid (2.83 mL) was heated at 80° C. for 4 hours. The reaction mixture was concentrated and triturated with acetone to afford 2-cinnolin-2-ium-2-ylacetate as a pale green solid.

¹H NMR (400 MHz, D₂O) 9.45 (d, 1H), 9.06 (d, 1H), 8.53-8.43 (m, 1H), 8.35-8.16 (m, 3H), 5.80 (s, 2H)

### Example 3: Preparation of isopropyl 2-(4,6,8-trimethylcinnolin-2-ium-2-yl)acetate chloride A6

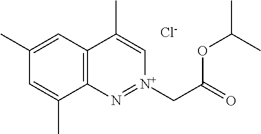 [SMILES: Cc1cc(C)c2n[n+](CC(=O)OC(C)C)cc(C)c2c1 ; [Cl-]]

### Step 1: Preparation of 2-(2-amino-3,5-dimethyl-phenyl)propan-2-ol

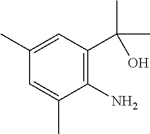

To a solution at −5° C. of methyl 2-amino-3,5-dimethyl-benzoate (1.95 g) in tetrahydrofuran (54.4 mL), under nitrogen atmosphere, was added methylmagnesium chloride (3M in tetrahydrofuran, 9.1 mL) drop wise over 10 minutes. The reaction was slowly warmed to room temperature. After 1.5 hours the reaction was cooled to 0° C. and further methylmagnesium chloride (3M in tetrahydrofuran, 9.1 mL) was added. The reaction was warmed to room temperature and stirred for 22 hours. The reaction mixture was quenched with saturated sodium bicarbonate solution and partially concentrated. The residue was diluted with ethyl acetate and the layers separated. The organic layer was washed with brine, dried over magnesium sulfate and concentrated to afford 2-(2-amino-3,5-dimethyl-phenyl)propan-2-ol as a green solid. The product was used without further purification.

¹H NMR (400 MHz, CDCl₃) 6.84 (d, 2H), 2.22 (s, 3H), 2.17-2.12 (m, 3H), 1.67 (s, 6H).

### Step 2: Preparation of 2-isopropenyl-4,6-dimethyl-aniline

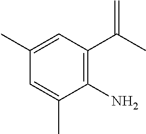

To a solution of 2-(2-amino-3,5-dimethyl-phenyl)propan-2-ol (1.9 g) in toluene (106 mL), under nitrogen atmosphere, was added p-toluenesulfonic acid monohydrate (0.19 g) and the mixture was heated at reflux under Dean-Stark conditions for 1.5 hours. The reaction mixture was quenched with saturated sodium bicarbonate solution and partially concentrated. The residue was diluted with ethyl acetate and the layers separated. The organic layer was washed with brine, dried over magnesium sulfate, concentrated and purified by chromatography on silica eluting with ethyl acetate in iso-hexane to give 2-isopropenyl-4,6-dimethyl-aniline as a dark yellow oil.

¹H NMR (400 MHz, CDCl₃) 6.80 (s, 1H), 6.74 (s, 1H), 5.29 (d, 1H), 5.03 (d, 1H), 3.68 (br. s., 2H), 2.24-2.13 (m, 6H), 1.57 (d, 3H).

### Step 3: Preparation of 4,6,8-trimethylcinnoline

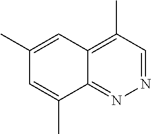

A mixture of 2-isopropenyl-4,6-dimethyl-aniline (0.5 g), water (2.64 mL) and concentrated sulfuric acid (0.535 mL) was cooled to 0° C. A solution of sodium nitrite (0.218 g) in water (3.16 mL) was added to the reaction drop wise over 10 mins, maintaining the temperature below 5° C. The reaction was allowed to warm to room temperature and stirred for 1 hour. The reaction mixture was basified using 2M aqueous sodium hydroxide under cooling and extracted with dichloromethane. The organic layer was dried over magnesium sulfate and concentrated to afford 4,6,8-trimethylcinnoline as a brown solid.

¹H NMR (400 MHz, CDCl₃) 9.10 (s, 1H), 7.56 (s, 1H), 7.47 (s, 1H), 2.99 (s, 3H), 2.64 (s, 3H), 2.55 (s, 3H).

### Step 4: Preparation of isopropyl 2-(4,6,8-trimethylcinnolin-2-ium-2-yl)acetate chloride A6

Isopropyl chloroacetate (0.102 g) was added dropwise to a solution of 4,6,8-trimethylcinnoline (0.1 g) in acetone (0.987 mL) and the reaction heated at 60° C. for 22 hours. The reaction mixture was concentrated to afford a red gum. The gum was dissolved in water and washed with dichloromethane. The aqueous layer was concentrated to afford isopropyl 2-(4,6,8-trimethylcinnolin-2-ium-2-yl)acetate chloride as a brown gum.

¹H NMR (400 MHz, D₂O) 9.21 (s, 1H), 7.94 (s, 1H), 7.86 (s, 1H), 5.82 (s, 2H), 5.06 (td, 1H), 2.81 (s, 3H), 2.71 (s, 3H), 2.62-2.52 (m, 3H), 1.19 (d, 6H)

### Example 4: Preparation of 2-(8-iodocinnolin-2-ium-2-yl)ethanesulfonic acid 2,2,2-trifluoroacetate A42

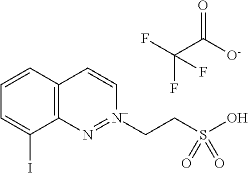 [SMILES: O=S(=O)(O)CC[n+]1ccc2cccc(I)c2n1 ; O=C([O-])C(F)(F)F]

A mixture of 8-iodocinnoline (0.1 g, prepared using the method reported by P. Knochel et. al., Org. Lett. 2014, 16, 1232-1235), sodium 2-bromoethanesulfonic acid (0.091 g) and water (1.25 mL) was heated to 100° C. for 20 hours. The reaction mixture was cooled to room temperature, diluted with water (1 mLL) and extracted with dichloromethane. The dichloromethane layer was discarded. The aqueous phase was concentrated and purified by preparative reverse phase HPLC (trifluoroacetic acid was present in the eluent) to give 2-(8-iodocinnolin-2-ium-2-yl)ethanesulfonic acid 2,2,2-trifluoroacetate as a yellow gum.

¹H NMR (400 MHz, CD₃OD) 9.82-9.77 (m, 1H), 9.13 (d, 1H), 8.92 (dd, 1H), 8.37 (dd, 1H), 8.02-7.94 (m, 1H), 5.53-5.47 (m, 2H), 3.80-3.73 (m, 2H) (SO₃H proton missing)

### Example 5: Preparation of 3-[4-(dichloromethyl)cinnolin-2-ium-2-yl]propane-1-sulfonate A39

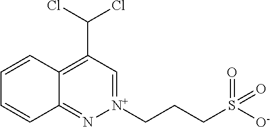 [SMILES: O=S(=O)([O-])CCC[n+]1cc(C(Cl)Cl)c2ccccc2n1]

### Step 1: Preparation of 4-(dichloromethyl)cinnoline

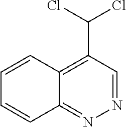 [SMILES: ClC(Cl)c1cnnc2ccccc12]

To a solution of 4-methylcinnoline (0.5 g) in carbon tetrachloride (15 mL) was added N-chlorosuccinimide (0.95 g) and benzoyl peroxide (0.025 g). The reaction was stirred at reflux for 30 mins then filtered, concentrated and purified by chromatography on silica eluting with ethyl acetate in iso-hexane to give 4-(dichloromethyl)cinnoline (0.333 g).

¹H NMR (400 MHz, CD₃OD) 9.60 (s, 1H), 8.62-8.49 (m, 2H), 8.08-7.99 (m, 2H), 7.99-7.85 (m, 1H). 4-(trichloromethyl)cinnoline, used to make compound A40, was also isolated in this reaction

¹H NMR (400 MHz, CD₃OD) 9.91 (s, 1H), 8.84-8.77 (m, 1H), 8.73-8.65 (m, 1H), 8.13-8.05 (m, 2H).

### Step 2: Preparation of 3-[4-(dichloromethyl)cinnolin-2-ium-2-yl]propane-1-sulfonate A39

To a solution of 4-(dichloromethyl)cinnoline (100 mg) in acetone (5 mL) was added oxathiolane 2,2-dioxide (2.21 g) and the mixture was stirred overnight at room temperature. The aqueous phase was concentrated and purified by preparative reverse phase HPLC to give 3-[4-(dichloromethyl)cinnolin-2-ium-2-yl]propane-1-sulfonate (0.035 mg) as a green solid.

¹H NMR (400 MHz, CD₃OD) 10.22 (s, 1H), 8.75 (d, 2H), 8.38-8.52 (m, 2H), 8.23 (s, 1H), 5.38 (t, 2H), 3.00 (t, 2H), 2.71 (m, 2H)

### Example 6: Preparation of methyl 4-cinnolin-2-ium-2-ylbutanoate 2,2,2-trifluoroacetate A16

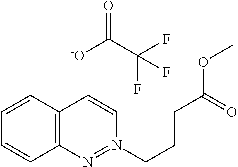

Methyl 4-bromobutanoate (0.426 g) was added to cinnoline (0.25 g) in 1,4-dioxane (3.84 mL) and stirred at 70° C. for 16 hours. The aqueous phase was concentrated and purified by preparative reverse phase HPLC (trifluoroacetic acid was present in the eluent) to give methyl 4-cinnolin-2-ium-2-ylbutanoate 2,2,2-trifluoroacetate (0.205 g) as dark blue gum.

¹H NMR (400 MHz, CD₃OD) 9.72 (d, 1H), 9.22 (d, 1H), 8.65-8.58 (m, 1H), 8.47-8.41 (m, 1H), 8.40-8.32 (m, 2H), 5.19 (t, 2H), 3.60 (s, 3H), 2.64-2.48 (m, 4H)

### Example 7: Preparation of 4-cinnolin-2-ium-2-ylbutane-2-sulfonate A43

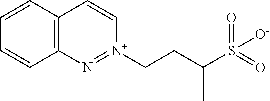 [SMILES: CC(CC[n+]1ccc2ccccc2n1)S(=O)(=O)[O-]]

To a solution of cinnoline (0.3 g) in N,N-dimethylformamide (5 mL) was added 3-methyloxathiolane 2,2-dioxide (0.471 g) and the mixture stirred at room temperature overnight. The resulting precipitate was filtered and triturated with acetone to give 4-cinnolin-2-ium-2-ylbutane-2-sulfonate as a grey solid.

¹H NMR (400 MHz, CD₃OD) 9.74 (d, 1H), 9.19 (d, 1H), 8.59-8.68 (m, 1H), 8.40-8.45 (m, 1H), 8.30-8.38 (m, 2H), 5.27-5.45 (m, 2H), 2.88-2.96 (m, 1H), 2.70 (dtd, 1H), 2.43-2.56 (m, 1H), 1.41 (d, 3H)

### Example 8: Preparation of N-[(1S)-2-cinnolin-2-ium-2-yl-1-methyl-ethyl]sulfamate A45

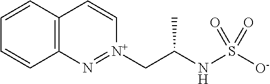 [SMILES: C[C@@H](C[n+]1ccc2ccccc2n1)NS(=O)(=O)[O-]]

### Step 1: Preparation of (4S)-4-methyloxathiazolidine 2,2-dioxide

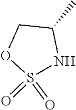

A mixture of trifluoroacetic acid (1.35 mL) and tert-butyl (4S)-4-methyl-2,2-dioxo-1,2,3-oxathiazolidine-3-carboxylate (0.16 g) was stirred for 1 hour at room temperature. The reaction mixture was concentrated to give (4S)-4-methyloxathiazolidine 2,2-dioxide, which was used without further purification.

### Step 2: Preparation of N-[(1S)-2-cinnolin-2-ium-2-yl-1-methyl-ethyl]sulfamate A45

To a solution of (4S)-4-methyloxathiazolidine 2,2-dioxide (0.089 g) in 1,2-dichloroethane (1.6 mL) was added cinnoline (0.07 g) and the mixture was heated at 80° C. for 1 hour. After cooling the precipitate was filtered and washed with acetone to afford N-[(1S)-2-cinnolin-2-ium-2-yl-1-methyl-ethyl]sulfamate as a grey solid.

¹H NMR (400 MHz, D₂O) 9.41 (d, 1H), 8.97 (d, 1H), 8.50-8.43 (m, 1H), 8.28-8.15 (m, 3H), 5.12 (dd, 1H), 4.79 (dd, 1H), 3.93 (ddd, 1H), 1.33 (d, 3H) (NH proton missing)

### Example 9: Preparation of 2-cinnolin-2-ium-2-yl-N-methylsulfonyl-acetamide bromide A15

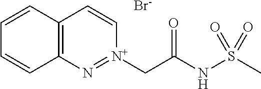 [SMILES: [Br-] ; CS(=O)(=O)NC(=O)C[n+]1ccc2ccccc2n1]

### Step 1: Preparation of 2-bromo-N-methylsulfonyl-acetamide

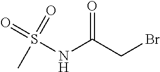

Methanesulfonamide (1 g) was dissolved in toluene (61.8 mL) and bromoacetyl bromide (8.49 g) was added drop wise at room temperature. The reaction was heated at 110° C. for 5 hours. The reaction was cooled and placed in an ice bath. The resulting precipitate was filtered, washed with cold toluene and dried to afford 2-bromo-N-methylsulfonyl-acetamide as a colourless solid.

¹H NMR (400 MHz, CDCl₃) 8.65 (br. s., 1H), 3.95 (s, 2H), 3.35 (s, 3H).

This method may be used to prepare:

3-bromo-N-methylsulfonyl-propanamide ¹H NMR (400 MHz, CDCl₃) 8.28 (br. s., 1H), 3.62 (t, 2H), 3.34 (s, 3H), 2.94 (t, 2H).

4-bromo-N-methylsulfonyl-butanamide ¹H NMR (400 MHz, CDCl₃) 8.64 (br. s., 1H), 3.50 (t, 2H), 3.39-3.25 (m, 3H), 2.56 (t, 2H), 2.23 (quin, 2H).

### Step 2: Preparation of 2-cinnolin-2-ium-2-yl-N-methylsulfonyl-acetamide bromide A15

To a solution of cinnoline (0.1 g) in acetone (1.54 mL) was added 2-bromo-N-methylsulfonyl-acetamide (0.183 g) and the mixture stirred at room temperature for 24 hours. The resulting precipitate was filtered and washed with acetone to afford 2-cinnolin-2-ium-2-yl-N-methylsulfonyl-acetamide bromide as a beige solid.

¹H NMR (400 MHz, D₂O) 9.45 (d, 1H), 9.09 (d, 1H), 8.53-8.44 (m, 1H), 8.35-8.22 (m, 3H), 5.92 (s, 2H), 3.17 (s, 3H) (NH proton missing)

### Example 10: Preparation of 2-(8-phenylcinnolin-2-ium-2-yl)ethanesulfonate A52

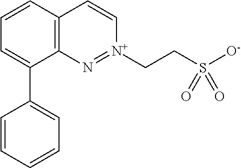 [SMILES: O=S(=O)([O-])CC[n+]1ccc2cccc(-c3ccccc3)c2n1]

### Step 1: Preparation of 8-Phenylcinnoline

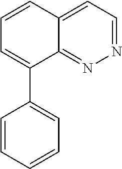 [SMILES: c1ccc(-c2cccc3ccnnc23)cc1]

A mixture of 8-iodocinnoline (0.52 g, prepared using the method reported by P. Knochel et. al., Org. Lett. 2014, 16, 1232-1235), phenylboronic acid (0.371 g), tripotassium phosphate (1.72 g) [1,1′-bis(diphenylphosphino)ferrocene]palladium(II) dichloride dichloromethane adduct (0.166 g), 1,2-dimethoxyethane (8.12 mL) and water (1.73 mL) was purged with nitrogen and heated at 120° C. under microwave irradiation for 30 minutes. The reaction mixture was partitioned between water and dichloromethane. The organic layer was dried over magnesium sulfate, concentrated and purified by chromatography on silica eluting with ethyl acetate in iso-hexane to give 8-Phenylcinnoline as a beige solid.

¹H NMR (400 MHz, CDCl₃) 9.36 (d, 1H), 7.92-7.77 (m, 6H), 7.58-7.42 (m, 3H).

### Step 2: Preparation of 2-(8-phenylcinnolin-2-ium-2-yl)ethanesulfonate A52

A mixture of 8-phenylcinnoline (0.3 g), sodium 2-bromoethanesulfonic acid (0.338 g) and water (4.66 mL) was heated at 100° C. overnight. Further sodium 2-bromoethanesulfonic acid (0.338 g) was added to the mixture and heating continued at 100° C. overnight. The reaction mixture was cooled, diluted with water (1 mL) and extracted with dichloromethane. The aqueous phase was concentrated and purified by preparative reverse phase HPLC to give 2-(8-phenylcinnolin-2-ium-2-yl)ethanesulfonate as a yellow solid.

¹H NMR (400 MHz, CD₃OD) 9.75-9.71 (d 1H), 9.20-9.16 (d, 1H), 8.41-8.28 (m, 3H), 7.81-7.74 (m, 2H), 7.63-7.49 (m, 3H), 5.42-5.35 (m, 2H), 3.66-3.59 (m, 2H).

### Example 11: Preparation of 2-(4-prop-1-ynylcinnolin-2-ium-2-yl)ethyl sulfate A54

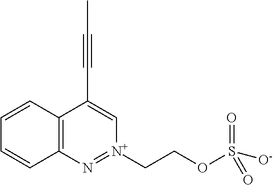 [SMILES: CC#Cc1c[n+](CCOS(=O)(=O)[O-])nc2ccccc12]

### Step 1: Preparation of 4-prop-1-ynylcinnoline

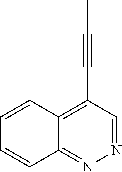

To a solution of 4-chlorocinnoline (0.5 g) in 1,4-dioxane (15.2 mL), under nitrogen atmosphere, was added tributyl(prop-1-ynyl)stannane (1.2 g) and palladium tetrakis triphenylphosphine (0.14 g). The reaction mixture was heated at 100° C. for 4 hours. The reaction mixture was cooled to room temperature, concentrated and purified by chromatography on silica eluting with ethyl acetate in iso-hexane to give 4-prop-1-ynylcinnoline as a yellow solid.

¹H NMR (400 MHz, CDCl₃) 9.26 (s, 1H), 8.51 (d, 1H), 8.21 (dd, 1H), 7.90-7.82 (m, 1H), 7.82-7.75 (m, 1H), 2.28 (s, 3H)

### Step 2: Preparation of 2-(4-prop-1-ynylcinnolin-2-ium-2-yl)ethyl sulfate A54

A solution of 4-prop-1-ynylcinnoline (0.2 g) and 1,3,2-dioxathiolane 2,2-dioxide (0.155 g) in 1,2-dichloroethane (2.38 mL) was stirred at room temperature overnight. The precipitate was collected by filtration, washed with acetone and dried to give 2-(4-prop-1-ynylcinnolin-2-ium-2-yl)ethyl sulfate as a green solid.

¹H NMR (400 MHz, CD₃OD) 9.77 (s, 1H), 8.62-8.55 (m, 2H), 8.38-8.29 (m, 2H), 5.34-5.29 (m, 2H), 4.69-4.63 (m, 2H), 2.43 (s, 3H)

### Example 12: Preparation of 2-cinnolin-2-ium-2-ylethyl(trifluoromethylsulfonyl)azanide A19

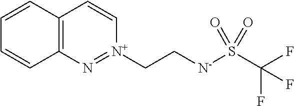 [SMILES: O=S(=O)([N-]CC[n+]1ccc2ccccc2n1)C(F)(F)F]

### Step 1: Preparation of N-(2-bromoethyl)-1,1,1-trifluoro-methanesulfonamide

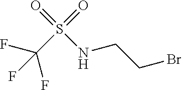 [SMILES: O=S(=O)(NCCBr)C(F)(F)F]

A mixture of 2-bromoethanamine bromide (1 g) and N-ethyldiisopropylamine (1.42 g) was stirred in dichloromethane (24.5 mL) at 0° C. until the reaction became homogeneous. Trifluoromethanesulfonic anhydride (1.55 g) was added drop wise and stirred at 0° C. for 3 hours. The reaction mixture was concentrated and partitioned between 1M aqueous hydrochloric acid and diethyl ether. The organic layer was washed with water, 1M aqueous hydrochloric acid, brine, dried over magnesium sulfate and concentrated to afford N-(2-bromoethyl)-1,1,1-trifluoro-methanesulfonamide as a pale yellow oil.

¹H NMR (400 MHz, CDCl₃) 5.44 (br. s., 1H), 3.71 (q, 2H), 3.53 (t, 2H).

### Step 2: Preparation of 2-cinnolin-2-ium-2-ylethyl(trifluoromethylsulfonyl)azanide A19

To a solution of cinnoline (0.1 g) in acetone (1.54 mL) was added N-(2-bromoethyl)-1,1,1-trifluoro-methanesulfonamide (0.236 g) and stirred at 60° C. for 18 hours. The reaction mixture was concentrated and partitioned between water and dichloromethane. The aqueous phase was concentrated and purified by preparative reverse phase HPLC to give N-(2-cinnolin-2-ium-2-ylethyl)-1,1,1-trifluoro-methanesulfonamide as a brown gum.

¹H NMR (400 MHz, CD₃OD) 9.76 (d, 1H), 9.30 (d, 1H), 8.70-8.64 (m, 1H), 8.51-8.45 (m, 1H), 8.43-8.37 (m, 2H), 5.32-5.22 (m, 2H), 4.08 (t, 2H)

### Example 13: Preparation of (2-cinnolin-2-ium-2-ylacetyl)-(dimethylsulfamoyl)azanide A21

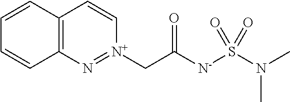 [SMILES: CN(C)S(=O)(=O)[N-]C(=O)C[n+]1ccc2ccccc2n1]

### Step 1: Preparation of 2-bromo-N-(dimethylsulfamoyl)acetamide

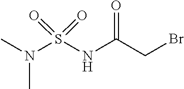

To a solution of dimethylsulfamide (0.5 g) and 4-(dimethylamino)pyridine (0.541 g) in dichloromethane (19.9 mL) at 0° C. was added bromoacetyl bromide (0.903 g) drop wise. The reaction was slowly warmed to room temperature and stirred for 24 hours. The reaction was partitioned with 0.5M aqueous hydrochloric acid. The organic layer was dried over magnesium sulfate and concentrated to afford 2-bromo-N-(dimethylsulfamoyl)acetamide as a pale yellow oil. The product was used without further purification.

### Step 2: Preparation of (2-cinnolin-2-ium-2-ylacetyl)-(dimethylsulfamoyl)azanide A21

To a solution of cinnoline (0.1 g) in acetone (1.54 mL) was added 2-bromo-N-(dimethylsulfamoyl)acetamide (0.226 g) and the mixture was stirred at room temperature for 23 hours. The reaction was concentrated and purified by preparative reverse phase HPLC to give (2-cinnolin-2-ium-2-ylacetyl)-(dimethylsulfamoyl)azanide as a dark green gum.

¹H NMR (400 MHz, CD₃OD) 9.72 (d, 1H), 9.32-9.29 (m, 1H), 8.67-8.63 (m, 1H), 8.51-8.46 (m, 1H), 8.44-8.38 (m, 2H), 6.11-6.05 (m, 2H), 2.94-2.91 (m, 6H)

### Example 14: Preparation of cinnolin-2-ium-2-ylmethyl hydrogen sulfate A31

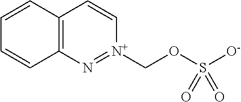 [SMILES: O=S(=O)([O-])OC[n+]1ccc2ccccc2n1]

To a stirred solution of cinnoline (0.1 g) in dichloroethane (3.07 mL) at room temperature was added N,N-dimethylformamide sulfur trioxide (0.127 g) giving a precipitate. The reaction was stirred for 30 minutes and paraformaldehyde (0.104 g) was added. The reaction was then heated at 90° C. for 3 hours. The precipitate was filtered off and washed with acetone to afford cinnolin-2-ium-2-ylmethyl hydrogen sulfate as a beige solid.

¹H NMR (400 MHz, D₂O) 9.68 (d, 1H), 9.11 (d, 1H), 8.60-8.50 (m, 1H), 8.35-8.19 (m, 3H), 6.63 (s, 2H)

### Example 15: Preparation of cinnolin-2-ium-2-ylmethyl(hydroxy)phosphinate chloride A73

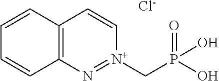 [SMILES: [Cl-] ; O=P(O)(O)C[n+]1ccc2ccccc2n1]

### Step 1: Preparation of 2-(diethoxyphosphorylmethyl)cinnolin-2-ium; 2,2,2-trifluoroacetate A122

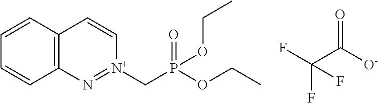 [SMILES: CCOP(=O)(C[n+]1ccc2ccccc2n1)OCC ; O=C([O-])C(F)(F)F]

To a stirred solution of diethyl hydroxymethylphosphonate (0.2 g) in dichloromethane (3.57 mL) at −78° C. under nitrogen was added N,N-diisopropylethylamine (0.188 g) followed by triflic anhydride (0.411 g). The reaction was warmed slowly to 0° C. over 2 hours. To this mixture at 0° C. was added a solution of cinnoline (0.12 g) in dichloromethane and the reaction was stirred at room temperature for 2 hours. The aqueous phase was concentrated and purified by preparative reverse phase HPLC to give 2-(diethoxyphosphorylmethyl)cinnolin-2-ium; 2,2,2-trifluoroacetate.

¹H NMR (400 MHz, CD₃OD) 9.70-9.67 (m, 1H), 9.27 (d, 1H), 8.76-8.29 (m, 4H), 5.88-5.80 (m, 2H), 4.21-4.03 (m, 4H), 1.31-1.19 (m, 6H).

### Step 2: Preparation of cinnolin-2-ium-2-ylmethylphosphonic Acid chloride A73

A mixture of 2-(diethoxyphosphorylmethyl)cinnolin-2-ium 2,2,2-trifluoroacetate (0.05 g) and 2M aqueous hydrochloric acid (0.51 mL) was heated at 80° C. for 23 hours. The reaction mixture was diluted with water and washed twice with dichloromethane. The aqueous layer was concentrated to afford cinnolin-2-ium-2-ylmethylphosphonic acid chloride as a brown gum.

¹H NMR (400 MHz, D₂O) 9.26-9.33 (m, 2H), 8.43-8.46 (m, 2H), 8.18-8.21 (m, 2H), 5.22 (d, 2H) (POH protons missing)

### Example 16: Preparation of 2-(3-methylcinnolin-2-ium-2-yl)ethanesulfonate A77

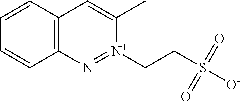 [SMILES: Cc1cc2ccccc2n[n+]1CCS(=O)(=O)[O-]]

### Step 1: Preparation of 1-(2-aminophenyl)propan-1-one

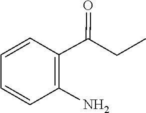

To a stirred solution of 2-aminobenzonitrile (15 g) in tetrahydrofuran (150 mL) at 0° C. was added ethylmagnesium chloride (2M in diethyl ether, 190.45 mL) drop wise. The reaction was allowed to warm to room temperature and stirred overnight. The reaction mixture was quenched with 2M aqueous hydrochloric acid and extracted with ethyl acetate (3×200 mL). The organic layers were dried over sodium sulfate and concentrated to give 1-(2-aminophenyl)propan-1-one, which was used without further purification.

¹H NMR (400 MHz, CDCl₃) 7.76 (d, 1H), 7.24-7.29 (m, 1H), 6.64-6.67 (m, 2H), 6.27 (br. s., 2H), 2.99 (q, 2H), 1.22 (t, 3H)

### Step 2: Preparation of 3-methylcinnolin-4-ol

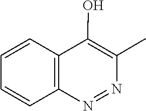

To a stirred solution of 1-(2-aminophenyl)propan-1-one (1 g) in acetic acid (5 mL) at 0° C. was added 2M aqueous hydrochloric acid (7 mL) drop wise. After one hour sodium nitrite (5.09 g) in water (5 mL) was added at 0° C. and stirred for a further hour. Urea (0.04 g) was added and stirred for another hour. A solution of sodium acetate (5.566 g) in water (10 mL) and dichloromethane (5 mL) was added at 0° C. and the mixture stirred for 12 hours at room temperature. The reaction mass was filtered and the solid washed with water (10 mL), dichloromethane (5 mL) and hexane (5 mL) to give 3-methylcinnolin-4-ol as a light brown solid.

¹H NMR (400 MHz, CDCl₃) 8.48-8.55 (m, 1H), 8.11-8.21 (m, 1H), 7.77-7.87 (m, 2H), 3.06 (s, 3H).

### Step 3: Preparation of 4-chloro-3-methyl-cinnoline

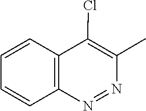

To a solution of 3-methylcinnolin-4-ol (5 g) in chlorobenzene (50 mL), under nitrogen atmosphere, was added 2-methylpyridine (0.581 g) followed by drop wise addition of phosphoryl chloride (4.41 mL) at room temperature. The mixture was then heated at reflux for 1 hour. The reaction was quenched in ice cold water and made alkaline with saturated aqueous sodium carbonate solution. The mixture was extracted with dichloromethane (3×50 mL) and the organic layers concentrated and purified by chromatography on silica eluting with ethyl acetate in iso-hexane to give 4-chloro-3-methyl-cinnoline.

¹H NMR (400 MHz, CDCl₃) 8.48 (s, 1H), 8.12 (s, 1H), 7.74-7.84 (m, 2H), 3.03 (s, 3H)

### Step 4: Preparation of 4-methyl-N′-(3-methylcinnolin-4-yl)benzenesulfonohydrazide

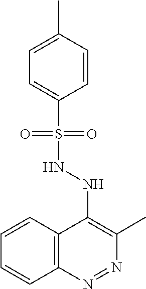 [SMILES: Cc1ccc(S(=O)(=O)NNc2c(C)nnc3ccccc23)cc1]

To a stirred solution of 4-chloro-3-methyl-cinnoline (8 g) in 1,2-dichloroethane (160 mL), under nitrogen atmosphere, was added 4-methylbenzenesulfonohydrazide (8.34 g) drop wise at room temperature and the mixture heated at 70° C. for 14 hour. The reaction mixture was cooled and the precipitate was filtered, washed with dichloromethane and dried to give 4-methyl-N′-(3-methylcinnolin-4-yl)benzenesulfonohydrazide.

¹H NMR (400 MHz, CD₃OD) 8.88-9.29 (m, 1H), 8.01-8.14 (m, 1H), 7.95 (s, 1H), 7.74 (d, 3H), 7.37 (d, 2H), 2.70 (s, 3H), 2.42 (s, 3H).

### Step 5: Preparation of 3-methylcinnoline

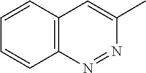

To a stirred solution of 4-methyl-N′-(3-methylcinnolin-4-yl)benzenesulfonohydrazide (14 g) in water (210 mL) was added a solution of sodium carbonate (12.23 g) in water (17 mL) drop wise at room temperature and this mixture was heated at 100° C. for 16 hours under nitrogen atmosphere. The reaction mixture was cooled and extracted with tert-butylmethylether (3×200 mL). The organic layers were concentrated and purified by chromatography on silica eluting with ethyl acetate in iso-hexane to give 3-methylcinnoline.

¹H NMR (400 MHz, CDCl₃) 8.49 (dd, 1H), 7.54-7.81 (m, 4H), 2.95 (s, 3H).

### Step 6: Preparation of 2-(3-methylcinnolin-2-ium-2-yl)ethanesulfonate A77

To a solution of sodium 2-bromoethanesulfonic acid (1.097 g) in water (10 mL) was added 3-methylcinnoline (500 mg) and the mixture heated at 100° C. under nitrogen atmosphere. Two further portions of sodium 2-bromoethanesulfonic acid (1.097 g) were added and heating continued for a total of 48 hours. The resulting precipitate was filtered, washed with acetone (5 mL), dichloromethane (5 mL) and tert-butylmethylether (5 mL). The solid was purified by preparative reverse phase HPLC to give 2-(3-methylcinnolin-2-ium-2-yl)ethanesulfonate.

¹H NMR (400 MHz, deuterium oxide) 8.93 (s, 1H), 8.48-8.50 (m, 1H), 8.21-8.24 (m, 3H), 5.42 (t, 2H), 3.86 (t, 2H), 3.20 (s, 3H).

### Example 17: Preparation of cinnolin-2-ium-2-ylmethanesulfonate A48

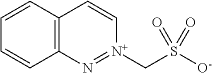 [SMILES: O=S(=O)([O-])C[n+]1ccc2ccccc2n1]

A stirred solution of 2-cinnolin-2-ium-2-ylacetate (0.15 g) in trimethylsilyl chlorosulfonate (2.39 mL) was heated at 80° C. for 18 hours. The reaction mixture was carefully quenched with water and purified by preparative reverse phase HPLC to give cinnolin-2-ium-2-ylmethanesulfonate as a pale blue solid.

¹H NMR (400 MHz, D₂O) 9.57 (d, 1H), 9.10 (d, 1H), 8.59-8.47 (m, 1H), 8.38-8.17 (m, 3H), 6.10 (s, 2H)

### Example 18: Preparation of 3-cinnolin-2-ium-2-ylpropyl(methoxy)phosphinate A72

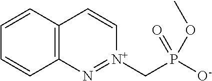 [SMILES: COP(=O)([O-])C[n+]1ccc2ccccc2n1]

### Step 1: Preparation of 1-bromo-3-dimethoxyphosphoryl-propane

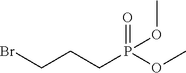

To a solution of dimethyl phosphite (5 g) in tetrahydrofuran (50 mL) was added 1,3-dibromopropane (4.8 mL) and potassium tert-butoxide (5.1 g) at room temperature and the mixture was stirred for 16 hours. The reaction mixture was poured onto ice and partitioned with ethyl acetate (2×300 mL). The combined organic layers were dried over sodium sulfate, concentrated and purified by chromatography on silica eluting with ethyl acetate in iso-hexane to give 1-bromo-3-dimethoxyphosphoryl-propane as a pale yellow oil.

¹H NMR (300 MHz, CDCl₃) 3.77 (s, 3H), 3.74 (s, 3H), 3.47 (t, 2H), 2.22-2.09 (m, 2H), 1.98-1.87 (m, 2H).

### Step 2: Preparation of 2-(3-dimethoxyphosphorylpropyl)cinnolin-2-ium bromide A123

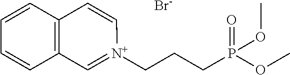 [SMILES: COP(=O)(CCC[n+]1ccc2ccccc2c1)OC ; [Br-]]

A solution of cinnoline (0.5 g) and 1-bromo-3-dimethoxyphosphoryl-propane (0.89 g) in N,N-dimethylformamide (50 mL) was stirred at room temperature for 48 hours. The reaction mixture was diluted with water (20 mL) and washed with dichloromethane (20 mL). The aqueous phase was concentrated and purified by preparative reverse phase HPLC to give 2-(3-dimethoxyphosphorylpropyl)cinnolin-2-ium bromide as a brown liquid.

¹H NMR (400 MHz, D₂O) 9.62-9.60 (d, 1H), 9.14-9.12 (d, 1H), 8.55-8.53 (m, 1H), 8.36-8.33 (m, 1H), 8.28-8.25 (m, 2H), 5.12-5.08 (t, 2H), 3.67-3.64 (d, 6H), 2.48-2.37 (m, 2H), 2.00-1.91 (m, 2H)

### Step 3: Preparation of 3-cinnolin-2-ium-2-ylpropyl(methoxy)phosphinate A72

A solution of 2-(3-dimethoxyphosphorylpropyl)cinnolin-2-ium bromide (0.1 g) in concentrated hydrochloric acid (10 mL) was stirred at room temperature for 18 hours. The reaction mixture was concentrated and purified by preparative reverse phase HPLC to give 3-cinnolin-2-ium-2-ylpropyl(methoxy)phosphinate as a brown liquid.

¹H NMR (400 MHz, D₂O) 9.51 (d, 1H), 9.05 (d, 1H), 8.55 (m, 1H), 8.33-8.20 (m, 3H), 5.12 (t, 2H), 3.50 (d, 3H), 2.42-2.30 (m, 2H), 1.75-1.66 (m, 2H)

### Example 19: Preparation of 7-fluoro-1H-cinnolin-4-one

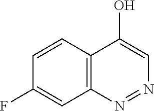 [SMILES: Oc1cnnc2cc(F)ccc12]

### Step 1: Preparation of 2-(2,4-difluorophenyl)-2-oxo-acetaldehyde

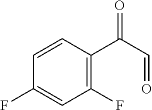 [SMILES: O=CC(=O)c1ccc(F)cc1F]

To a solution of 1-(2,4-difluorophenyl)ethanone (10 g) in 1,4-dioxane (150 mL) was added selenium dioxide (7.75 g) at room temperature followed by water (5 mL). The reaction mixture was heated at reflux for 8 hours. The reaction was filtered through celite and thoroughly washed with ethyl acetate (150 mL). The combined filtrate was dried over anhydrous sodium sulfate and concentrated to give 2-(2,4-difluorophenyl)-2-oxo-acetaldehyde as pale yellow liquid, which was used in the next step without further purification.

### Step 2: Preparation of tert-butyl N-[[2-(2,4-difluorophenyl)-2-oxo-ethylidene]amino]carbamate

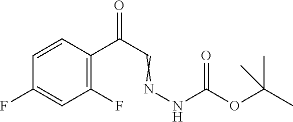 [SMILES: CC(C)(C)OC(=O)NN=CC(=O)c1ccc(F)cc1F]

To a suspension of 2-(2,4-difluorophenyl)-2-oxo-acetaldehyde (12 g) in methanol (120 mL) was added water (120 mL) followed by acetic acid (15 mL) at room temperature. To this a solution of tert-butyl carbazate (6.52 g) in methanol (30 mL) was slowly added at room temperature over 15 minutes. The resulting reaction mixture was stirred at room temperature for 12 hours. The resulting precipitate was isolated by filtration and air dried to give tert-butyl N-[[2-(2,4-difluorophenyl)-2-oxo-ethylidene]amino]carbamate as an orange solid, which was used in the next step without further purification.

### Step 3: Preparation of 7-fluorocinnolin-4-ol

To a solution of tert-butyl N-[[2-(2,4-difluorophenyl)-2-oxo-ethylidene]amino]carbamate (16 g) in N,N-dimethylformamide (100 mL) was added potassium carbonate (15.54 g) at room temperature. The mixture was heated at 110° C. for 8 hours, cooled to room temperature and poured into ice water (300 mL). The aqeuous mixture was neutralized to pH 5 to 6 by addition of 1M aqueous hydrochloric acid and extracted with dichloromethane (3×300 mL). The combined organic layers were washed with brine (300 mL), dried over anhydrous sodium sulfate and concentrated. The concentrate was co-evaporated with toluene to give 7-fluorocinnolin-4-ol as an off-white solid.

¹H NMR (400 MHz, CDCl₃) 9.35-9.34 (d, 1H), 8.17-8.14 (dd, 1H), 7.91-7.86 (m, 2H), 7.60-7.55 (m, 1H)

This compound can be taken through to compound, A83, following an equivalent or related methods as used in Example 16.

### Example 20: Preparation of 2-cinnolin-2-ium-2-ylethanesulfonate A24

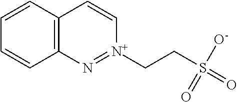 [SMILES: O=S(=O)([O-])CC[n+]1ccc2ccccc2n1]

A mixture of cinnoline (0.2 g), sodium 2-bromoethanesulfonate (0.364 g) and water (4.61 mL) was heated at 100° C. overnight. The reaction mixture was concentrated and triturated with acetone. The resulting solid was filtered off and purified by preparative reverse phase HPLC to give 2-cinnolin-2-ium-2-ylethanesulfonate as a green solid.

¹H NMR (400 MHz, CD₃OD) 9.74 (d, 1H), 9.16 (d, 1H), 8.65-8.59 (m, 1H), 8.43-8.30 (m, 3H), 5.50-5.44 (m, 2H), 3.74-3.67 (m, 2H)

### Example 21: Preparation of 2-(8-methoxycarbonylcinnolin-2-ium-2-yl)ethanesulfonate A109

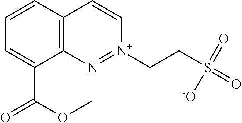

### Step 1: Preparation of methyl-2-amino-3-bromo-benzoate

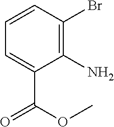

To a solution of 2-amino-3-bromo-benzoic acid (50 g) in methanol (500 mL) was added conc. sulfuric acid drop wise at room temperature. The mixture was heated at 100° C. for 16 hours. The reaction mixture was concentrated, diluted with water (500 mL), cooled to ˜0° C. and slowly neutralized with solid sodium hydrogen carbonate. The aqueous layer was extracted with ethyl acetate (3×500 mL). The combined organic layers were washed with brine (100 mL), dried over anhydrous sodium sulfate and concentrated to afford methyl-2-amino-3-bromo-benzoate as an off-white solid, which was used without further purification.

### Step 2: Preparation of methyl-2-amino-3-iodo-benzoate

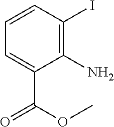

A mixture of methyl-2-amino-3-bromo-benzoate (10 g), 1,4-dioxane (100 mL), sodium iodide (12.9 g) and copper (I) iodide (413 mg) in a sealed tube was purged with argon for 15 minutes. To this was added 1,2-Dimethylethylenediamine (7.5 mL) and the reaction mixture was then heated at 110° C. for 16 hours. The reaction mixture was poured into water (150 mL) and extracted with ethyl acetate (2×250 mL). The combined organic layers were washed with brine (100 mL), dried over anhydrous sodium sulfate and concentrated to afford methyl-2-amino-3-iodo-benzoate as a pale yellow liquid, which was used without further purification.

### Step 3: Preparation of methyl 2-amino-3-(2-trimethylsilylethynyl)benzoate

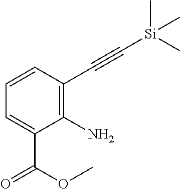 [SMILES: COC(=O)c1cccc(C#C[Si](C)(C)C)c1N]

A mixture of methyl-2-amino-3-iodo-benzoate (6.8 g), trimethylsilyl-acetylene (10.4 mL), copper (I) iodide (0.233 g) and triethylamine (21 mL) in acetonitrile (70 mL) was purged with argon for 10 minutes. To this was added bis(triphenylphosphine)palladium chloride (0.86 g) and the reaction mixture was stirred at room temperature for 3 hours. The reaction mixture was poured in to water (100 mL) and extracted with ethyl acetate (2×100 mL). The combined organic layers were dried over anhydrous sodium sulfate and concentrated to afford methyl 2-amino-3-(2-trimethylsilylethynyl)benzoate as a brown liquid, which was used without further purification.

### Step 4: Preparation of methyl 2-(diethylaminoazo)-3-(2-trimethylsilylethynyl)benzoate

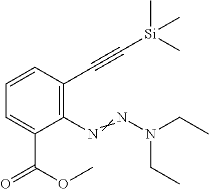 [SMILES: CCN(CC)N=Nc1c(C#C[Si](C)(C)C)cccc1C(=O)OC]

To a mixture of methyl 2-amino-3-(2-trimethylsilylethynyl)benzoate (5.4 g), tetrahydrofuran (27 mL), acetonitrile (27 mL) and water (34 mL), cooled to −5° C., was added conc. hydrochloric acid (11 mL). To this was added slowly a solution of sodium nitrite (3 g) in water (11 mL) over 15 minutes, maintaining the temperature at −5° C. The resulting reaction mixture was added to a cooled (˜0° C.) solution of diethylamine (23 mL) and potassium carbonate (18.1 g) in water (135 mL). The reaction mixture was stirred at ˜0° C. for 30 minutes and then at room temperature for 1 hour. The reaction mixture was extracted with ethyl acetate (2×100 mL). The combined organic layers were dried over anhydrous sodium sulfate, concentrated and purified by chromatography on silica eluting with ethyl acetate in iso-hexane to give methyl 2-(diethylaminoazo)-3-(2-trimethylsilylethynyl)benzoate as a brown liquid.

### Step 5: Preparation of methyl 2-(diethylaminoazo)-3-ethynyl-benzoate

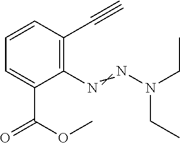 [SMILES: C#Cc1cccc(C(=O)OC)c1N=NN(CC)CC]

To a solution of methyl 2-(diethylaminoazo)-3-(2-trimethylsilylethynyl)benzoate (7 g) in tetrahydrofuran (70 mL) at ˜0° C. was added tetra n-butyl ammonium fluoride (1M in tetrahydrofuran, 13.5 mL) drop wise. The reaction mixture was warmed to room temperature and stirred for 3 hours. The reaction mixture was diluted with water (200 mL) and extracted with ethyl acetate (2×100 mL). The combined organic layers were dried over anhydrous sodium sulfate, concentrated and purified by chromatography on silica eluting with ethyl acetate in iso-hexane to give methyl 2-(diethylaminoazo)-3-ethynyl-benzoate as a brown solid.

### Step 6: Preparation of methyl cinnoline-8-carboxylate

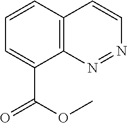

A solution of methyl 2-(diethylaminoazo)-3-ethynyl-benzoate (1 g) in 1,2-dichlorobenzene (5 mL) in a sealed tube was heated at 200° C. for 30 minutes. The reaction mixture was concentrated and purified by chromatography on silica eluting with ethyl acetate in iso-hexane to give methyl cinnoline-8-carboxylate as a dark brown solid.

¹H NMR (400 MHz, D₂O) 9.50 (d, 1H), 8.33 (d, 1H), 8.25 (d, 1H), 8.15 (d, 1H), 7.95 (t, 1H), 4.00 (s, 3H)

### Step 7: Preparation of 2-(8-methoxycarbonylcinnolin-2-ium-2-yl)ethanesulfonate A109

A solution of methyl cinnoline-8-carboxylate (0.1 g) and sodium 2-bromoethanesulfonic acid (0.1 g) in water (5 mL) was heated at 100° C. for 16 hours. The reaction mixture was concentrated and purified by preparative reverse phase HPLC to give 2-(8-methoxycarbonylcinnolin-2-ium-2-yl)ethanesulfonate as a dark brown solid.

¹H NMR (400 MHz, D₂O) 9.69 (d, 1H), 9.16 (d, 1H), 8.69 (d, 1H), 8.50 (d, 1H), 8.30 (t, 1H), 5.4 (t, 2H), 4.06 (s, 3H), 3.87 (t, 2H)

### Example 22: Preparation of 2-[8-(dimethylcarbamoyl)cinnolin-2-ium-2-yl]ethanesulfonate A102

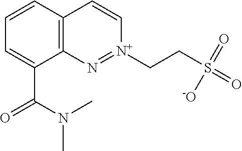

### Step 1: Preparation of cinnoline-8-carboxylic Acid

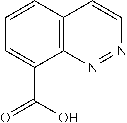 [SMILES: O=C(O)c1cccc2ccnnc12]

To a solution of methyl cinnoline-8-carboxylate (1 g) in tetrahydrofuran (15 mL) was added a solution of lithium hydroxide monohydrate (0.45 g) in water (4 mL) at room temperature. The reaction mixture was stirred at room temperature for 3 hours. The reaction mixture was concentrated, dissolved in water and the pH adjusted with dilute hydrochloric acid to pH 5. The resulting solid was filtered off and dried to afford cinnoline-8-carboxylic acid as a light brown solid.

¹H NMR (400 MHz, DMSO-d₆) 14.60 (s, 1H), 9.56 (d, 1H), 8.48 (d, 1H), 8.40 (d, 1H), 8.34 (d, 1H), 8.02 (t, 1H)

### Step 2: Preparation of N,N-dimethylcinnoline-8-carboxamide

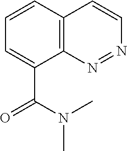

To a solution of cinnoline-8-carboxylic acid (0.2 g) in N,N-dimethylformamide (15 mL), cooled to ˜0° C., was added 1-[bis(dimethylamino)methylene]-1H-1,2,3-triazolo[4,5-b]pyridinium 3-oxide hexafluorophosphate (1.6 g) and N,N-diisopropylethylamine (1.6 mL). The reaction mixture was stirred at ˜0° C. for 10 minutes and then dimethylamine hydrochloride (1.14 g) was added. The reaction mixture was warmed to room temperature and stirred for 16 hours. The reaction mixture was concentrated and purified by chromatography on silica eluting with a mixture of methanol and dichloromethane to give N,N-dimethylcinnoline-8-carboxamide as a brown solid.

¹H NMR (400 MHz, D₂O) 9.45 (d, 1H), 8.29 (d, 1H), 8.13 (d, 1H), 7.92 (t, 1H), 7.84 (d, 1H), 3.15 (s, 3H), 2.70 (s, 3H)

### Step 3: Preparation of 2-[8-(dimethylcarbamoyl)cinnolin-2-ium-2-yl]ethanesulfonate A102

A solution of N,N-dimethylcinnoline-8-carboxamide (0.3 g) and sodium 2-bromoethanesulfonic acid (0.283 g) in water (8 mL) was heated at 100° C. for 16 hours. The reaction mixture was concentrated and purified by preparative reverse phase HPLC to give 2-[8-(dimethylcarbamoyl)cinnolin-2-ium-2-yl]ethanesulfonate as a light yellow solid.

¹H NMR (400 MHz, D₂O) 9.61 (d, 1H), 9.10 (d, 1H), 8.35 (d, 1H), 8.27 (d, 1H), 8.21 (t, 1H), 5.36 (t, 2H), 3.67 (t, 2H), 3.16 (s, 3H), 2.76 (s, 3H)

### Example 23: Preparation of 2-(8-cyanocinnolin-2-ium-2-yl)ethanesulfonate A110

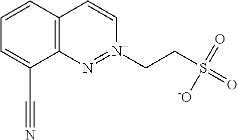

### Step 1: Preparation of cinnoline-8-carboxamide

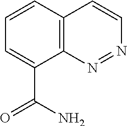 [SMILES: NC(=O)c1cccc2ccnnc12]

In a sealed tube methyl cinnoline-8-carboxylate (0.5 g) was dissolved in methanolic ammonia (7M solution in methanol, 40 mL) at room temperature. The reaction mixture was heated at 70° C. for 3 hours. The reaction mixture was cooled to room temperature and the resulting precipitate was filtered off to afford cinnoline-8-carboxamide as a brown solid.

¹H NMR (400 MHz, D₂O) 9.52 (d, 1H), 8.52 (dd, 1H), 8.40 (d, 1H), 8.26 (dd, 1H), 7.99 (t, 1H) (NH protons missing)

### Step 2: Preparation of cinnoline-8-carbonitrile

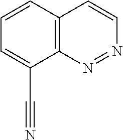 [SMILES: N#Cc1cccc2ccnnc12]

To a mixture of cinnoline-8-carboxamide (0.3 g) and pyridine (0.2 mL) in dichloromethane (30 mL) was added dichlorophosphorylbenzene (0.26 mL) drop wise at room temperature over 10 minutes. The reaction mixture was stirred at room temperature for 16 hours. The reaction mixture was poured onto ice and neutralised with aqueous sodium bicarbonate (100 mL) and extracted with dichloromethane (200 mL). The organic layer was dried over anhydrous sodium sulfate, concentrated and purified by chromatography on silica eluting with a mixture of ethyl acetate and iso-hexane to give cinnoline-8-carbonitrile as an off-white solid.

¹H NMR (400 MHz, D₂O) 9.61 (d, 1H), 8.61 (d, 1H), 8.47-8.43 (m, 2H), 8.03 (t, 1H)

### Step 3: Preparation of 2-(8-cyanocinnolin-2-ium-2-yl)ethanesulfonate A110

A solution of cinnoline-8-carbonitrile (0.18 g) and sodium 2-bromoethanesulfonic acid (0.22 g) in water (8 mL) was heated at 100° C. for 16 hours. The reaction mixture was concentrated and purified by preparative reverse phase HPLC to give 2-(8-cyanocinnolin-2-ium-2-yl)ethanesulfonate as an off-white solid.

¹H NMR (400 MHz, D₂O) 9.77 (d, 1H), 9.23 (d, 1H), 8.77 (d, 1H), 8.57 (d, 1H), 8.30 (t, 1H), 5.5 (t, 2H), 3.81 (t, 2H)

### Example 24: Preparation of 2-(8-acetamidocinnolin-2-ium-2-yl)ethanesulfonate A107

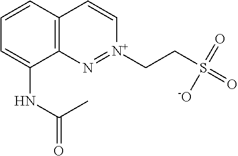

### Step 1: Preparation of cinnolin-8-amine

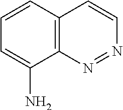 [SMILES: Nc1cccc2ccnnc12]

To a solution of cinnoline-8-carboxylic acid (0.4 g) in 1,4-dioxane (8 mL) at room temperature was added triethylamine (0.48 mL) and diphenylphosphoryl azide (0.54 mL) drop wise over 10 minutes. The reaction mixture was stirred at room temperature for 1 hour. Water (8 mL) was added and the reaction mixture was heated at 100° C. for 48 hours. The reaction mixture was poured into water (100 mL) and extracted with ethyl acetate (2×200 mL). The combined organic layers were dried over anhydrous sodium sulfate, concentrated and purified by chromatography on silica eluting with ethyl acetate in iso-hexane to give cinnolin-8-amine as a dark yellow liquid.

¹H NMR (400 MHz, DMSO-d₆) 9.20 (d, 1H), 7.73 (d, 1H), 7.53 (t, 1H), 7.08 (d, 1H), 6.94 (d, 1H), 5.43 (brs, 2H)

### Step 2: Preparation of N-cinnolin-8-ylacetamide

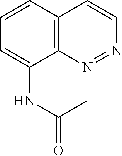

To a solution of cinnolin-8-amine (0.08 g) and pyridine (4 mL) in dichloromethane (4 mL) cooled to ˜0° C. was added acetyl chloride (0.04 mL). The reaction mixture was then stirred at room temperature for 3 hours. The reaction mixture was poured into cold water (25 mL) and extracted with ethyl acetate (50 mL). The combined organic layers were dried over anhydrous sodium sulfate, concentrated and purified by chromatography on silica eluting with ethyl acetate in iso-hexane to give N-cinnolin-8-ylacetamide as a light-yellow semi solid.

¹H NMR (400 MHz, DMSO-d₆) 10.10 (s, 1H), 9.34 (d, 1H), 8.87 (d, 1H), 7.88 (d, 1H), 7.76 (d, 1H), 7.50 (d, 1H), 2.39 (s, 3H)

### Step 3: Preparation of 2-(8-acetamidocinnolin-2-ium-2-yl)ethanesulfonate A107

A solution of N-cinnolin-8-ylacetamide (0.05 g) and sodium 2-bromoethanesulfonic acid (0.05 g) in water (8 mL) was heated at 100° C. for 16 hours. The reaction mixture was concentrated and purified by preparative reverse phase HPLC to give 2-(8-acetamidocinnolin-2-ium-2-yl)ethanesulfonate as an orange brown solid.

¹H NMR (400 MHz, D₂O) 9.32 (d, 1H), 8.99 (d, 1H), 8.73 (d, 1H), 8.21 (t, 1H), 8.0 (d, 1H), 5.41 (t, 2H), 3.77 (t, 2H), 2.76 (s, 3H) (NH proton missing)

### Example 25: Preparation of (3S)-4-cinnolin-2-ium-2-yl-3-hydroxy-butanoate A74

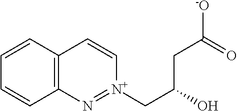

### Step 1: Preparation of (3S)-4-cinnolin-2-ium-2-yl-3-hydroxy-butanenitrile 2,2,2-trifluoroacetate

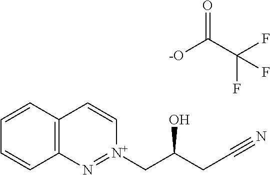

To a mixture of cinnoline (0.15 g) and (S)-4-chloro-3-hydroxybutyronitrile (0.16 g) in acetone (3 mL) was added sodium iodide (0.19 g) and the mixture was stirred at room temperature for an hour. The reaction was then stirred at 60° C. for 60 hours. The reaction mixture was concentrated and purified by preparative reverse phase HPLC (trifluoroacetic acid was present in the eluent) to give (3S)-4-cinnolin-2-ium-2-yl-3-hydroxy-butanenitrile 2,2,2-trifluoroacetate as a gum.

¹H NMR (400 MHz, CD₃OD) 9.69 (d, 1H), 9.24 (d, 1H), 8.68-8.61 (m, 1H), 8.49-8.44 (m, 1H), 8.42-8.33 (m, 2H), 5.32 (dd, 1H), 5.12 (dd, 1H), 4.62 (dddd, 1H), 4.13-4.01 (m, 2H) (OH proton missing)

### Step 2: Preparation of (3S)-4-cinnolin-2-ium-2-yl-3-hydroxy-butanoate A74

A mixture of (3S)-4-cinnolin-2-ium-2-yl-3-hydroxy-butanenitrile 2,2,2-trifluoroacetate (0.105 g) and 2M aqueous hydrochloric acid (1.28 mL) was heated at 80° C. for an hour. The mixture was concentrated and the resulting residue was crystallised from methanol and dichloromethane to afford (3S)-4-cinnolin-2-ium-2-yl-3-hydroxy-butanoate as a colourless solid.

¹H NMR (400 MHz, CD₃OD) 9.71-9.61 (m, 1H), 9.23 (d, 1H), 8.71-8.61 (m, 1H), 8.51-8.43 (m, 1H), 8.42-8.32 (m, 2H), 5.33 (dd, 1H), 5.11 (dd, 1H), 4.75-4.63 (m, 1H), 4.25-4.12 (m, 2H) (OH proton missing)

### Example 26: Preparation of 3-(cinnolin-2-ium-2-ylmethyl)benzenesulfonic Acid A93

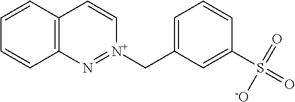

A mixture of cinnoline (0.08 g) and 3-(bromomethyl)benzenesulfonic acid (0.17 g) in acetone (2 mL) was stirred at room temperature for 24 hours. The reaction mixture was concentrated and purified by preparative reverse phase HPLC to give 3-(cinnolin-2-ium-2-ylmethyl)benzenesulfonic acid as an orange gum/foam.

¹H NMR (400 MHz, CD₃OD) 9.84 (d, 1H), 9.21 (d, 1H), 8.55-8.61 (m, 1H), 8.38-8.44 (m, 1H), 8.28-8.36 (m, 2H), 8.02 (t, 1H), 7.86 (dt, 1H), 7.72 (dt, 1H), 7.50 (t, 1H), 6.35 (s, 2H)

### Example 27: Preparation of 3-cinnolin-2-ium-2-yl-2,2-dimethyl-propanoic Acid 2,2,2-trifluoroacetate A113

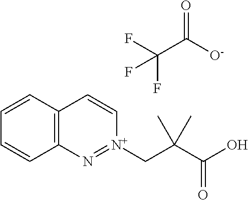 [SMILES: O=C([O-])C(F)(F)F ; CC(C)(C[n+]1ccc2ccccc2n1)C(=O)O]

A mixture of cinnolin-2-ium tetrafluoroborate (0.2 g), 3-hydroxy-2,2-dimethylpropanoic acid (0.553 g) and triphenylphosphine (0.486 g) were dissolved in acetonitrile (9.18 mL). To this mixture was added diisopropyl azodicarboxylate (0.369 mL) drop wise. The reaction was stirred at room temperature overnight and then heated at reflux for 7 hours. The mixture was cooled overnight then partitioned between water and ether. The aqueous layer was concentrated and purified by preparative reverse phase HPLC (trifluoroacetic acid was present in the eluent) to give 3-cinnolin-2-ium-2-yl-2,2-dimethyl-propanoic acid 2,2,2-trifluoroacetate as a green oil.

¹H NMR (400 MHz, CD₃OD) 9.74 (d, 1H), 9.23 (d, 1H), 8.63-8.53 (m, 1H), 8.47-8.34 (m, 3H), 5.31 (s, 2H), 1.39 (s, 6H) (C02H proton missing)

### Example 28: Preparation of 3-cinnolin-2-ium-2-ylbutanoic Acid 2,2,2-trifluoroacetate A115

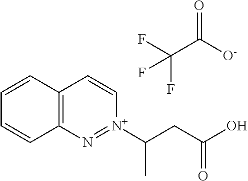 [SMILES: CC(CC(=O)O)[n+]1ccc2ccccc2n1 ; O=C([O-])C(F)(F)F]

A mixture of cinnoline (0.2 g), (E)-but-2-enoic acid (0.397 g), water (1 mL) and glacial acetic acid (1 mL) was then heated at 180° C. under microwave irradiation for 60 minutes. The reaction mixture was concentrated and purified by preparative reverse phase HPLC (trifluoroacetic acid was present in the eluent) to give 3-cinnolin-2-ium-2-ylbutanoic acid 2,2,2-trifluoroacetate as an orange oil.

¹H NMR (400 MHz, CD₃OD) 9.88-9.72 (m, 1H), 9.21 (dd, 1H), 8.68-8.59 (m, 1H), 8.46-8.32 (m, 3H), 5.85-5.48 (m, 1H), 3.58-3.18 (m, 2H), 1.84 (d, 3H) (C02H proton missing)

### Example 29: Preparation of (2S)-2-amino-3-cinnolin-2-ium-2-yl-propanoic Acid 2,2,2-trifluoroacetate A23

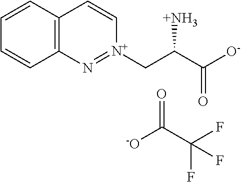

A mixture of N-(tert-butoxycarbonyl)-l-serine beta-lactone (0.158 g) and cinnoline (0.1 g) in acetone (3.84 mL) was stirred at room temperature for 24 hours. The reaction mixture was concentrated and stirred in trifluoroacetic acid (0.768 mL) for 1 hour. After concentration the residue was purified by preparative reverse phase HPLC (trifluoroacetic acid was present in the eluent) to give (2S)-2-amino-3-cinnolin-2-ium-2-yl-propanoic acid 2,2,2-trifluoroacetate as a brown gum.

¹H NMR (400 MHz, D₂O) 9.52 (d, 1H), 9.09 (d, 1H), 8.55-8.49 (m, 1H), 8.33-8.23 (m, 3H), 5.57 (d, 2H), 3.98-3.89 (m, 1H) (NH protons missing)

### Example 30: Preparation of [(1R)-1-carboxy-3-cinnolin-2-ium-2-yl-propyl]ammonium 2,2,2-trifluoroacetate A12

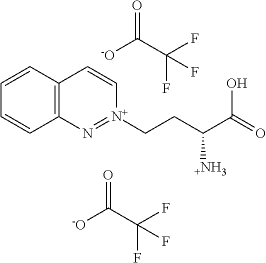

### Step 1: Preparation of benzyl (2S)-2-(benzyloxycarbonylamino)-4-hydroxy-butanoate

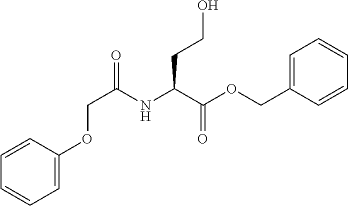 [SMILES: O=C(COc1ccccc1)N[C@@H](CCO)C(=O)OCc1ccccc1]

A solution of (3S)-4-benzyloxy-3-(benzyloxycarbonylamino)-4-oxo-butanoic acid (5 g) in tetrahydrofuran (75 mL) was cooled to −10° C. To this solution was added 4-methylmorpholine (1.73 mL) followed by ethyl carbonochloridate (1.471 mL) and the reaction was stirred at −10° C. for 10 minutes. A solution of sodium borohydride (1.62 g) in water (10 mL) was added cautiously and the reaction stirred at ˜0° C. for a further 30 minutes. The reaction mixture was partitioned between water and ether. The aqueous was extracted with further ether (2×). The combined organic layers were dried over magnesium sulfate, concentrated and purified by chromatography on silica eluting with ethyl acetate in iso-hexane to give benzyl (2S)-2-(benzyloxycarbonylamino)-4-hydroxy-butanoate.

¹H NMR (400 MHz, CD₃OD) 7.39-7.28 (m, 10H), 5.22-5.03 (m, 4H), 4.47-4.36 (m, 1H), 3.69-3.57 (m, 2H), 2.12-1.99 (m, 1H), 1.86 (dt, 1H) (OH proton missing)

### Step 2: Preparation of benzyl (2S)-2-(benzyloxycarbonylamino)-4-iodo-butanoate

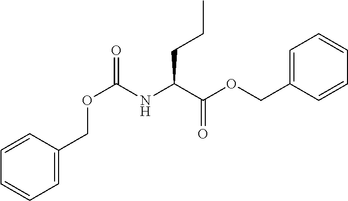 [SMILES: CCC[C@H](NC(=O)OCc1ccccc1)C(=O)OCc1ccccc1]

A mixture of benzyl (2S)-2-(benzyloxycarbonylamino)-4-hydroxy-butanoate (3.894 g), triphenylphosphine (4.53 g) and imidazole (1.235 g) in tetrahydrofuran (70 mL) was cooled to ˜0° C. To this solution was added iodine (4.317 g) in portions and the reaction was stirred at ˜0° C. for 2 hours. The reaction mixture was quenched with aqueous sodium thiosulfate and extracted with ether. The organic layer was washed with water, dried over magnesium sulfate, concentrated and purified by chromatography on silica eluting with ethyl acetate in iso-hexane to give benzyl (2S)-2-(benzyloxycarbonylamino)-4-iodo-butanoate as a white solid.

¹H NMR (400 MHz, CDCl₃) 7.40-7.25 (m, 10H), 5.47-5.11 (m, 4H), 4.51-4.36 (m, 1H), 3.17-3.06 (m, 2H), 2.50-2.34 (m, 1H), 2.34-2.13 (m, 1H)

### Step 3: Preparation of benzyl (2R)-2-(benzyloxycarbonylamino)-4-cinnolin-2-ium-2-yl-butanoate iodide

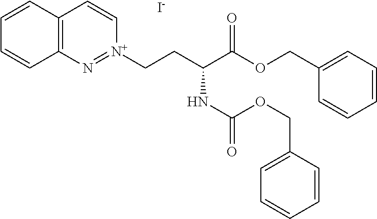 [SMILES: O=C(N[C@H](CC[n+]1ccc2ccccc2n1)C(=O)OCc1ccccc1)OCc1ccccc1 ; [I-]]

Benzyl (2S)-2-(benzyloxycarbonylamino)-4-iodo-butanoate (0.383 g) was added to a solution of cinnoline (0.1 g) in 1,4-dioxane (1.54 mL) and the mixture heated at 70° C. for 28 hours. The reaction mixture was concentrated and partitioned between water and dichloromethane. The organic layer was concentrated to give benzyl (2R)-2-(benzyloxycarbonylamino)-4-cinnolin-2-ium-2-yl-butanoate iodide which was used in the next step without further purification.

### Step 4: Preparation of [(1R)-1-carboxy-3-cinnolin-2-ium-2-yl-propyl]ammonium 2,2,2-trifluoroacetate A12

A mixture of benzyl (2R)-2-(benzyloxycarbonylamino)-4-cinnolin-2-ium-2-yl-butanoate iodide (0.448 g) and 2M aqueous hydrochloric acid (3.07 mL) was heated at 80° C. for 1 hour. The reaction mixture was cooled and washed with dichloromethane. The aqueous layer was concentrated and the residue was purified by preparative reverse phase HPLC (trifluoroacetic acid was present in the eluent) to give [(1R)-1-carboxy-3-cinnolin-2-ium-2-yl-propyl]ammonium 2,2,2-trifluoroacetate as a brown gum.

¹H NMR (400 MHz, D₂O) 9.50 (d, 1H), 9.03 (d, 1H), 8.56-8.44 (m, 1H), 8.34-8.12 (m, 3H), 5.32-5.21 (m, 2H), 4.01 (t, 1H), 2.77 (dq, 2H) (NH and CO₂H protons missing)

Additional compounds in Table A (below) were prepared by analogues procedures, from appropriate starting materials. The skilled person would understand that the compounds of formula (I) may exist as an agronomically acceptable salt, a zwitterion or an agronomically acceptable salt of a zwitterion as described hereinbefore. Where mentioned the specific counterion is not considered to be limiting, and the compound of formula (I) may be formed with any suitable counter ion.

NMR spectra contained herein were recorded on either a 400 MHz Bruker AVANCE III HD equipped with a Bruker SMART probe unless otherwise stated. Chemical shifts are expressed as ppm downfield from TMS, with an internal reference of either TMS or the residual solvent signals. The following multiplicities are used to describe the peaks: s=singlet, d=doublet, t=triplet, dd=double doublet, dt=double triplet, q=quartet, quin=quintet, m=multiplet. Additionally br. is used to describe a broad signal and app. is used to describe and apparent multiplicity.

TABLE A
Physical Data for Compounds of the Invention

| Compound | | |
|---|---|---|
| Number | Structure | ¹H NMR (400 MHz, unless stated) |
| A1 | 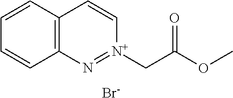 | (DMSO-d₆) 9.91 (d, 1H), 9.51 (d, 1H), 8.67 (d, 1H), 8.56 (d, 1H), 8.44 (br. s., 2H), 6.25 (br. s., 2H), 3.81 (s, 3H) |
| A2 | 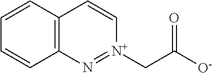 [SMILES: O=C([O-])C[n+]1ccc2ccccc2n1] | (D₂O) 9.45 (d, 1H), 9.06 (d, 1H), 8.53- 8.43 (m, 1H), 8.35-8.16 (m, 3H), 5.80 (s, 2H) |
| A3 | 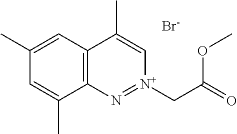 | (CD₃OD) 9.54 (s, 1H), 8.16 (s, 1H), 8.05 (s, 1H), 4.87 (s, 3H), 3.89 (s, 2H), 2.99 (s, 3H), 2.88 (s, 3H), 2.76 (s, 3H) |
| A4 | 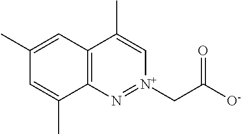 [SMILES: Cc1cc(C)c2n[n+](CC(=O)[O-])cc(C)c2c1] | (D₂O) 9.15 (s, 1H), 7.93 (s, 1H), 7.85 (s, 1H), 5.59 (s, 2H), 2.80 (s, 3H), 2.72 (s, 3H), 2.57 (s, 3H) |
| A5 | 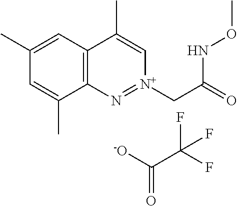 | (D₂O) 9.22 (s, 1H), 7.90 (s, 1H), 7.82 (s, 1H), 5.64 (s, 2H), 3.71 (s, 3H), 2.80 (s, 3H), 2.68 (s, 3H), 2.56 (s, 3H), (NH proton missing) |
| A6 | 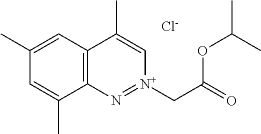 [SMILES: Cc1cc(C)c2n[n+](CC(=O)OC(C)C)cc(C)c2c1 ; [Cl-]] | (D₂O) 9.21 (s, 1H), 7.94 (s, 1H), 7.86 (s, 1H), 5.82 (s, 2H), 5.06 (td, 1H), 2.81 (s, 3H), 2.71 (s, 3H), 2.62-2.52 (m, 3H), 1.19 (d, 6H) |
| A7 | 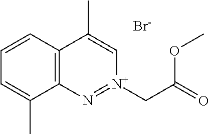 [SMILES: COC(=O)C[n+]1cc(C)c2cccc(C)c2n1 ; [Br-]] | (D₂O) 9.34 (s, 1H), 8.21-8.15 (m, 1H), 8.14-8.08 (m, 1H), 8.01 (d, 1H), 5.94 (s, 2H), 3.77 (s, 3H), 2.89 (s, 3H), 2.77 (s, 3H) |
| A8 | 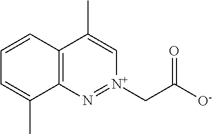 [SMILES: Cc1c[n+](CC(=O)[O-])nc2c(C)cccc12] | (D₂O) 9.29 (s, 1H), 8.18-8.12 (m, 1H), 8.07 (t, 1H), 7.97 (d, 1H), 5.72 (s, 2H), 2.86 (s, 3H), 2.76 (s, 3H) |
| A9 | 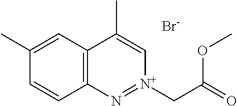 | (D₂O) 9.27 (s, 1H), 8.31 (d, 1H), 8.16 (s, 1H), 8.07 (dd, 1H), 5.86 (s, 2H), 3.76 (s, 3H), 2.86 (s, 3H), 2.66 (s, 3H) |
| A10 | 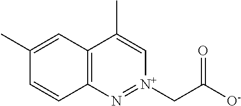 [SMILES: Cc1ccc2n[n+](CC(=O)[O-])cc(C)c2c1] | (D₂O) 9.23 (s, 1H), 8.28 (d, 1H), 8.12 (s, 1H), 8.03 (dd, 1H), 5.65 (s, 2H), 2.84 (s, 3H), 2.64 (s, 3H) |
| A11 | 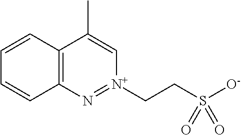 [SMILES: Cc1c[n+](CCS(=O)(=O)[O-])nc2ccccc12] | (D₂O) 9.43 (s, 1H), 8.41-8.49 (m, 1H), 8.28-8.39 (m, 1H), 8.10-8.25 (m, 2H), 5.24-5.40 (m, 2H), 3.61-3.80 (m, 2H), 2.91 (s, 3H) |
| A12 | 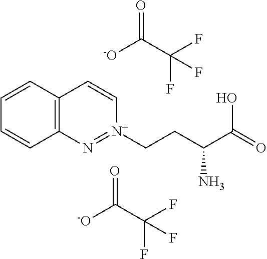 | (D₂O) 9.50 (d, 1H), 9.03 (d, 1H), 8.56- 8.44 (m, 1H), 8.34-8.12 (m, 3H), 5.32- 5.21 (m, 2H), 4.01 (t, 1H), 2.77 (dq, 2H) (NH and CO2H protons missing) |
| A13 | 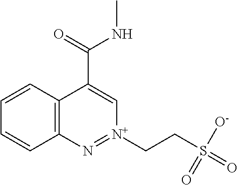 [SMILES: CNC(=O)c1c[n+](CCS(=O)(=O)[O-])nc2ccccc12] | (D₂O) 9.78 (s, 1H), 8.63 (d, 1H), 8.45 (d, 1H), 8.29-8.40 (m, 2H), 5.41- 5.51 (m, 2H), 3.76-3.86 (m, 2H), 3.04 (s, 3H) (NH proton missing) |
| A14 | 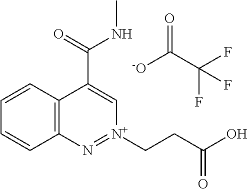 | (D₂O) 9.81 (s, 1H), 8.66-8.56 (m, 1H), 8.48-8.41 (m, 1H), 8.40-8.30 (m, 2H), 5.40 (t, 2H), 3.43 (t, 2H), 3.07 (s, 3H) (NH and CO2H protons missing) |
| A15 | 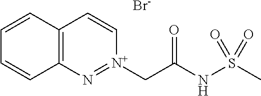 [SMILES: [Br-] ; CS(=O)(=O)NC(=O)C[n+]1ccc2ccccc2n1] | (D₂O) 9.45 (d, 1H), 9.09 (d, 1H), 8.53- 8.44 (m, 1H), 8.35-8.22 (m, 3H), 5.92 (s, 2H), 3.17 (s, 3H) (NH proton missing) |
| A16 | 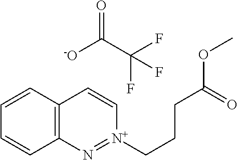 | (CD₃OD) 9.72 (d, 1H), 9.22 (d, 1H), 8.65-8.58 (m, 1H), 8.47-8.41 (m, 1H), 8.40-8.32 (m, 2H), 5.19 (t, 2H), 3.60 (s, 3H), 2.64-2.48 (m, 4H) |
| A17 | 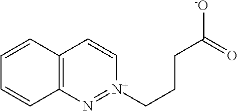 | (D₂O) 9.48 (d, 1H), 9.01 (d, 1H), 8.53- 8.44 (m, 1H), 8.31-8.18 (m, 3H), 5.08 (t, 2H), 2.57-2.37 (m, 4H) |
| A18 | 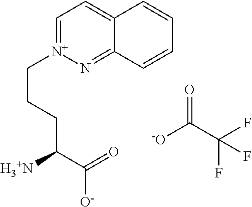 | (D₂O) 9.49-9.44 (m, 1H), 9.05-9.00 (m, 1H), 8.53-8.47 (m, 1H), 8.30-8.19 (m, 3H), 5.13-5.01 (m, 2H), 3.97-3.86 (m, 1H), 2.41-1.91 (m, 4H), (NH protons missing) |
| A19 | 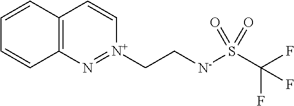 [SMILES: O=S(=O)([N-]CC[n+]1ccc2ccccc2n1)C(F)(F)F] | (CD₃OD) 9.76 (d, 1H), 9.30 (d, 1H), 8.70-8.64 (m, 1H), 8.51-8.45 (m, 1H), 8.43-8.37 (m, 2H), 5.32-5.22 (m, 2H), 4.08 (t, 2H) |
| A20 | 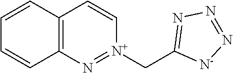 [SMILES: c1ccc2n[n+](Cc3nnn[n-]3)ccc2c1] | (CD₃OD) 9.91 (d, 1H), 9.30 (d, 1H), 8.69-8.55 (m, 1H), 8.41-8.35 (m, 3H), 6.72 (s, 2H) |
| A21 | 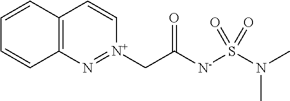 [SMILES: CN(C)S(=O)(=O)[N-]C(=O)C[n+]1ccc2ccccc2n1] | (CD₃OD) 9.72 (d, 1H), 9.32-9.29 (m, 1H), 8.67-8.63 (m, 1H), 8.51-8.46 (m, 1H), 8.44-8.38 (m, 2H), 6.11-6.05 (m, 2H), 2.94-2.91 (m, 6H) |
| A22 | 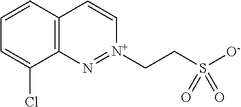 [SMILES: O=S(=O)([O-])CC[n+]1ccc2cccc(Cl)c2n1] | (300 MHz, D₂O) 9.68-9.58 (m, 1H), 9.14-9.04 (m, 1H), 8.40-8.31 (m, 1H), 8.27-8.10 (m, 2H), 5.53-5.41 (m, 2H), 3.87-3.75 (m, 2H) |
| A23 | 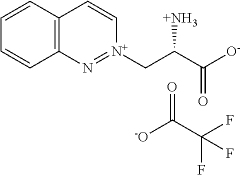 | (D₂O) 9.52 (d, 1H), 9.09 (d, 1H), 8.55- 8.49 (m, 1H), 8.33-8.23 (m, 3H), 5.57 (d, 2H), 3.98-3.89 (m, 1H), (NH protons missing) |
| A24 | 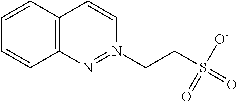 [SMILES: O=S(=O)([O-])CC[n+]1ccc2ccccc2n1] | (CD₃OD) 9.74 (d, 1H), 9.16 (d, 1H), 8.65-8.59 (m, 1H), 8.43-8.30 (m, 3H), 5.50-5.44 (m, 2H), 3.74-3.67 (m, 2H) |
| A25 | 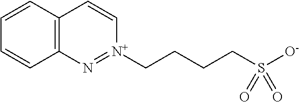 [SMILES: O=S(=O)([O-])CCCC[n+]1ccc2ccccc2n1] | (D₂O) 9.47 (d, 1H), 9.01 (d, 1H), 8.53- 8.46 (m, 1H), 8.30-8.17 (m, 3H), 5.07 (t, 2H), 2.96-2.87 (m, 2H), 2.35- 2.24 (m, 2H), 1.85-1.69 (m, 2H) |
| A26 | 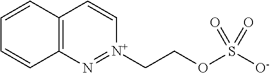 [SMILES: O=S(=O)([O-])OCC[n+]1ccc2ccccc2n1] | (D₂O) 9.49 (d, 1H), 9.04 (d, 1H), 8.54- 8.47 (m, 1H), 8.32-8.20 (m, 3H), 5.35- 5.30 (m, 2H), 4.69-4.65 (m, 2H) |
| A27 | 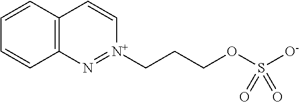 [SMILES: O=S(=O)([O-])OCCC[n+]1ccc2ccccc2n1] | (D₂O) 9.48 (d, 1H), 9.00 (d, 1H), 8.52- 8.44 (m, 1H), 8.30-8.19 (m, 3H), 5.17 (t, 2H), 4.12-4.05 (m, 2H), 2.61-2.50 (m, 2H) |
| A28 | 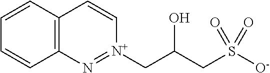 [SMILES: O=S(=O)([O-])CC(O)C[n+]1ccc2ccccc2n1] | (D₂O) 9.45 (d, 1H), 9.03 (d, 1H), 8.57- 8.44 (m, 1H), 8.33-8.14 (m, 3H), 5.34 (dd, 1H), 5.06 (dd, 1H), 4.73 (dt, 1H), 3.36-3.27 (m, 1H), 3.24-3.10 (m, 1H) (OH protons missing) |
| A29 | 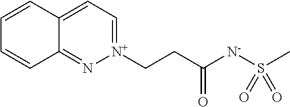 [SMILES: CS(=O)(=O)[N-]C(=O)CC[n+]1ccc2ccccc2n1] | (CD₃OD) 9.78 (d, 1H), 9.21 (d, 1H), 8.65-8.57 (m, 1H), 8.47-8.41 (m, 1H), 8.39-8.33 (m, 2H), 5.42 (t, 2H), 3.43- 3.37 (m, 2H), 3.20 (s, 3H) |
| A30 | 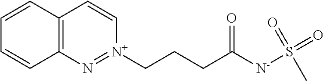 [SMILES: CS(=O)(=O)[N-]C(=O)CCC[n+]1ccc2ccccc2n1] | (CD₃OD) 9.72 (d, 1H), 9.21 (d, 1H), 8.66-8.57 (m, 1H), 8.47-8.41 (m, 1H), 8.40-8.31 (m, 2H), 5.19 (t, 2H), 3.11 (s, 3H), 2.66-2.51 (m, 4H) |
| A31 | 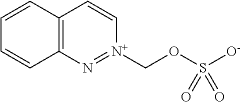 [SMILES: O=S(=O)([O-])OC[n+]1ccc2ccccc2n1] | (D₂O) 9.68 (d, 1H), 9.11 (d, 1H), 8.60- 8.50 (m, 1H), 8.35-8.19 (m, 3H), 6.63 (s, 2H) |
| A32 | 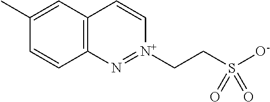 [SMILES: Cc1ccc2n[n+](CCS(=O)(=O)[O-])ccc2c1] | (D₂O) 9.33-9.43 (m, 1H), 8.81 (d, 1H), 8.35 (d, 1H), 8.08 (dd, 1H), 7.96-8.03 (m, 1H), 5.27-5.35 (m, 2H), 3.66-3.76 (m, 2H), 2.57-2.67 (m, 3H) |
| A33 | 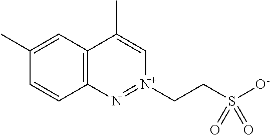 [SMILES: Cc1ccc2n[n+](CCS(=O)(=O)[O-])cc(C)c2c1] | (D₂O) 9.08-9.25 (m, 1H), 8.34-8.48 (m, 1H), 8.20-8.27 (m, 1H), 8.08-8.18 (m, 1H), 5.49-5.60 (m, 2H), 3.60-3.73 (m, 2H), 2.89-3.01 (m, 3H), 2.55-2.65 (m, 3H) |
| A34 | 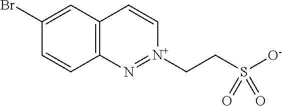 [SMILES: O=S(=O)([O-])CC[n+]1ccc2cc(Br)ccc2n1] | (D₂O) 9.52 (d, 1H), 8.91 (d, 1H), 8.52 (d, 1H), 8.25-8.39 (m, 2H), 5.28-5.40 (m, 2H), 3.65-3.77 (m, 2H) |
| A35 | 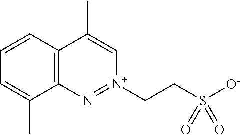 [SMILES: Cc1c[n+](CCS(=O)(=O)[O-])nc2c(C)cccc12] | (D₂O) 9.37 (s, 1H), 7.89-8.26 (m, 3H), 5.16-5.39 (m, 2H), 3.69-3.81 (m, 2H), 2.84-2.90 (m, 3H), 2.73-2.82 (m, 3H) |
| A36 | 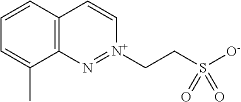 [SMILES: Cc1cccc2cc[n+](CCS(=O)(=O)[O-])nc12] | (D₂O) 9.46 (d, 1H), 8.92 (d, 1H), 7.99- 8.13 (m, 3H), 5.27-5.44 (m, 2H), 3.68- 3.86 (m, 2H), 2.85 (d, 3H) |
| A37 | 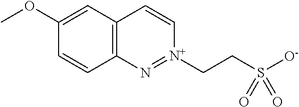 [SMILES: COc1ccc2n[n+](CCS(=O)(=O)[O-])ccc2c1] | (D₂O) 9.20 (d, 1H), 8.63 (d, 1H), 8.32 (d, 1H), 7.75-7.83 (m, 1H), 7.50 (d, 1H), 5.16-5.28 (m, 2H), 3.99-4.07 (m, 3H), 3.59-3.74 (m, 2H) |
| A38 | 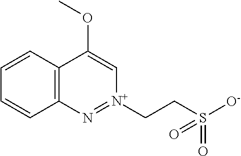 [SMILES: COc1c[n+](CCS(=O)(=O)[O-])nc2ccccc12] | (D₂O) 9.37 (s, 1H), 8.38 (ddt, 2H), 8.05-8.19 (m, 2H), 5.31 (t, 2H), 4.22- 4.34 (m, 3H), 3.67-3.79 (m, 2H) |
| A39 | 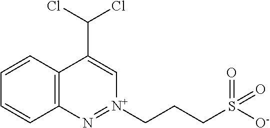 [SMILES: O=S(=O)([O-])CCC[n+]1cc(C(Cl)Cl)c2ccccc2n1] | (CD₃OD) 10.22 (s, 1H), 8.75 (d, 2H), 8.38-8.52 (m, 2H), 8.23 (s, 1H), 5.38 (t, 2H), 3.00 (t, 2H), 2.71 (m, 2H) |
| A40 | 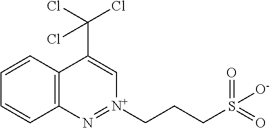 [SMILES: O=S(=O)([O-])CCC[n+]1cc(C(Cl)(Cl)Cl)c2ccccc2n1] | (CD₃OD) 10.40 (s, 1H), 9.10 (d, 1H), 8.84 (d, 1H), 8.53-8.60 (m, 1H), 8.41- 8.48 (m, 1H), 5.44 (t, 2H), 3.02 (t, 2H), 2.73 (quin, 2H) |
| A41 | 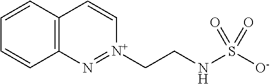 [SMILES: O=S(=O)([O-])NCC[n+]1ccc2ccccc2n1] | (D₂O) 9.43 (d, 1H), 8.99 (d, 1H), 8.51- 8.45 (m, 1H), 8.30-8.18 (m, 3H), 5.15- 5.08 (m, 2H), 3.71-3.64 (m, 2H) (NH proton missing) |
| A42 | 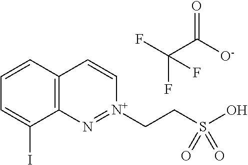 [SMILES: O=S(=O)(O)CC[n+]1ccc2cccc(I)c2n1 ; O=C([O-])C(F)(F)F] | (CD₃OD) 9.82-9.77 (m, 1H), 9.13 (d, 1H), 8.92 (dd, 1H), 8.37 (dd, 1H), 8.02- 7.94 (m, 1H), 5.53-5.47 (m, 2H), 3.80- 3.73 (m, 2H) (SO3H proton missing) |
| A43 | 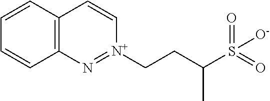 [SMILES: CC(CC[n+]1ccc2ccccc2n1)S(=O)(=O)[O-]] | (CD₃OD) 9.74 (d, 1H), 9.19 (d, 1H), 8.59-8.68 (m, 1H), 8.40-8.45 (m, 1H), 8.30-8.38 (m, 2H), 5.27-5.45 (m, 2H), 2.88-2.96 (m, 1H), 2.70 (dtd, 1H), 2.43-2.56 (m, 1H), 1.41 (d, 3H) |
| A44 | 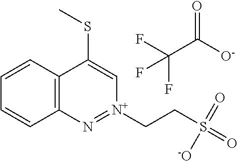 | (D₂O) 9.21 (s, 1H), 8.34 (d, 1H), 8.18- 8.26 (m, 1H), 8.03-8.16 (m, 2H), 5.29 (t, 2H), 3.72 (t, 2H), 2.79 (s, 3H) (SO3H proton missing) |
| A45 | 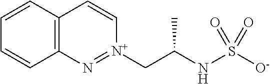 [SMILES: C[C@@H](C[n+]1ccc2ccccc2n1)NS(=O)(=O)[O-]] | (D₂O) 9.41 (d, 1H), 8.97 (d, 1H), 8.50- 8.43 (m, 1H), 8.28-8.15 (m, 3H), 5.12 (dd, 1H), 4.79 (dd, 1H), 3.93 (ddd, 1H), 1.33 (d, 3H) (NH proton missing) |
| A46 | 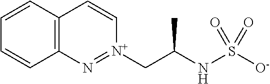 [SMILES: C[C@H](C[n+]1ccc2ccccc2n1)NS(=O)(=O)[O-]] | (D₂O) 9.40 (d, 1H), 8.96 (d, 1H), 8.50- 8.43 (m, 1H), 8.28-8.13 (m, 3H), 5.11 (dd, 1H), 4.83-4.75 (m, 1H), 3.98-3.84 (m, 1H), 1.32 (d, 3H) (NH proton missing) |
| A47 | 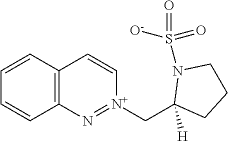 [SMILES: O=S(=O)([O-])N1CCC[C@H]1C[n+]1ccc2ccccc2n1] | (D₂O) 9.37 (d, 1H), 8.95 (d, 1H), 8.49- 8.43 (m, 1H), 8.29-8.15 (m, 3H), 5.10 (dd, 1H), 4.81 (dd, 1H), 4.22-4.09 (m, 1H), 3.28-3.12 (m, 2H), 1.96-1.67 (m, 4H) |
| A48 | 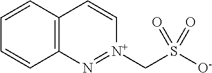 [SMILES: O=S(=O)([O-])C[n+]1ccc2ccccc2n1] | (D₂O) 9.57 (d, 1H), 9.10 (d, 1H), 8.59- 8.47 (m, 1H), 8.38-8.17 (m, 3H), 6.10 (s, 2H) |
| A49 | 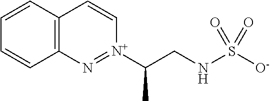 [SMILES: C[C@H](CNS(=O)(=O)[O-])[n+]1ccc2ccccc2n1] | (D₂O) 9.41 (d, 1H), 8.95 (d, 1H), 8.54- 8.42 (m, 1H), 8.31-8.09 (m, 3H), 5.41- 5.27 (m, 1H), 3.54 (d, 2H), 1.71 (d, 3H) (NH proton missing) |
| A50 | 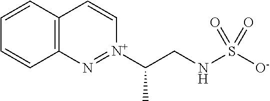 [SMILES: C[C@@H](CNS(=O)(=O)[O-])[n+]1ccc2ccccc2n1] | (D₂O) 9.40 (d, 1H), 8.94 (d, 1H), 8.39- 8.53 (m, 1H), 8.04-8.28 (m, 3H), 5.26- 5.43 (m, 1H), 3.53 (d, 2H), 1.71 (d, 3H) (NH proton missing) |
| A51 | 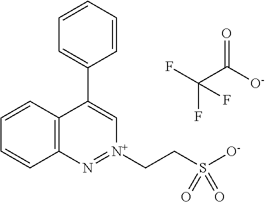 [SMILES: O=S(=O)([O-])CC[n+]1cc(-c2ccccc2)c2ccccc2n1 ; O=C([O-])C(F)(F)F] | (D₂O) 9.61 (s, 1H), 8.56-8.49 (m, 1H), 8.31-8.25 (m, 1H), 8.24-8.13 (m, 2H), 7.68-7.59 (m, 5H), 5.39 (t, 2H), 3.79- 3.74 (t, 2H) (SO3H proton missing) |
| A52 | 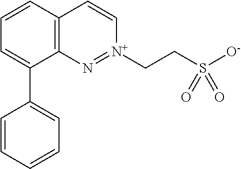 [SMILES: O=S(=O)([O-])CC[n+]1ccc2cccc(-c3ccccc3)c2n1] | (CD₃OD) 9.75-9.71 (d 1H), 9.20-9.16 (d, 1H), 8.41-8.28 (m, 3H), 7.81-7.74 (m, 2H), 7.63-7.49 (m, 3H), 5.42-5.35 (m, 2H), 3.66-3.59 (m, 2H) |
| A53 | 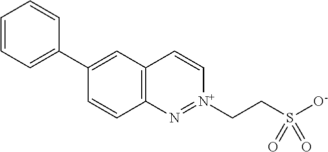 [SMILES: O=S(=O)([O-])CC[n+]1ccc2cc(-c3ccccc3)ccc2n1] | (CD₃OD) 9.67 (d, 1H), 9.11 (d, 1H), 8.73-8.63 (m, 3H), 8.02-7.98 (m, 2H), 7.67-7.57 (m, 3H), 5.44 (t, 2H), 3.70 (t, 2H) |
| A54 | 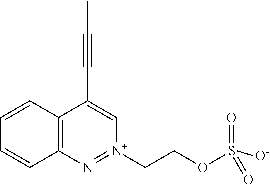 [SMILES: CC#Cc1c[n+](CCOS(=O)(=O)[O-])nc2ccccc12] | (CD₃OD) 9.77 (s, 1H), 8.62-8.55 (m, 2H), 8.38-8.29 (m, 2H), 5.34-5.29 (m, 2H), 4.69-4.63 (m, 2H), 2.43 (s, 3H) |
| A55 | 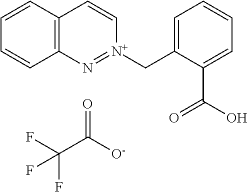 [SMILES: O=C(O)c1ccccc1C[n+]1ccc2ccccc2n1 ; O=C([O-])C(F)(F)F] | (CD₃OD) 9.76 (d, 1H), 9.18 (d, 1H), 8.45-8.51 (m, 1H), 8.39-8.43 (m, 1H), 8.27-8.36 (m, 2H), 8.20 (d, 1H), 7.74- 7.77 (m, 2H), 7.63-7.70 (m, 1H), 6.63 (s, 2H) (CO2H proton missing) |
| A56 | 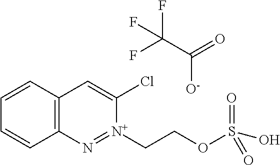 [SMILES: O=S(=O)(O)OCC[n+]1nc2ccccc2cc1Cl ; O=C([O-])C(F)(F)F] | (D₂O) 9.15 (s, 1H), 8.50-8.46 (m, 1H), 8.25-8.18 (m, 3H), 5.52-5.47 (m, 2H), (2 protons hidden under water signal, SO4H proton missing) Isolated as a 2:1 mixture with N1 alkylated compound |
| A57 | 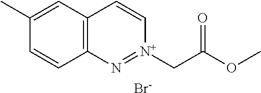 | (CD₃OD) 9.54 (d, 1H), 9.03 (d, 1H), 8.40 (dd, 1H), 8.18-8.15 (m, 2H), 5.96 (s, 2H), 3.78 (s, 3H), 2.70 (s, 3H) |
| A58 | 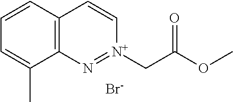 [SMILES: COC(=O)C[n+]1ccc2cccc(C)c2n1 ; [Br-]] | (DMSO-d₆) 9.89 (d, 1H), 9.45 (d, 1H), 8.38-8.34 (m, 2H), 8.25 (d, 1H), 6.24 (s, 2H), 3.82 (s, 3H), 2.68 (s, 3H) |
| A59 | 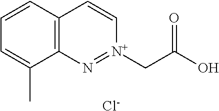 | (D₂O) 9.43 (d, 1H), 9.01 (d, 1H), 8.17- 8.12 (m, 2H), 8.07 (m, 1H), 5.86 (s, 2H), 2.84 (s, 3H) (CO2H proton missing) |
| A60 | 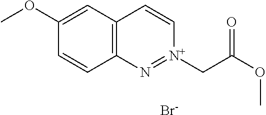 | (300 MHz, D₂O) 9.17 (d, 1H), 8.71 (d, 1H), 8.32 (d, 1H), 7.83 (dd, 1H), 7.55 (d, 1H), 5.82 (s, 2H), 4.07 (s, 3H), 3.77 (s, 3H) |
| A61 | 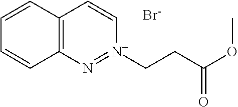 [SMILES: COC(=O)CC[n+]1ccc2ccccc2n1 ; [Br-]] | (CD₃OD) 9.79 (d, 1H), 9.22 (d, 1H), 8.60 (m, 1H), 8.44 (m, 1H), 8.37-8.35 (m, 2H), 5.40 (t, 2H), 3.68 (s, 3H), 3.40 (t, 2H) |
| A62 | 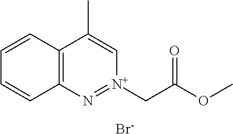 [SMILES: COC(=O)C[n+]1cc(C)c2ccccc2n1 ; [Br-]] | (DMSO-d₆) 9.93 (s, 1H), 8.64-8.61 (m, 2H), 8.46-8.38 (m, 2H), 6.20 (s, 2H), 3.81 (s, 3H), 3.03 (s, 3H) |
| A63 | 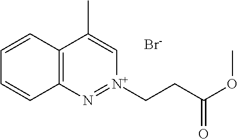 [SMILES: COC(=O)CC[n+]1cc(C)c2ccccc2n1 ; [Br-]] | (DMSO-d₆) 10.03 (s, 1H), 8.58-8.56 (m, 2H), 8.40-8.30 (m, 2H), 5.30 (t, 2H), 3.63 (s, 3H), 3.37 (t, 2H), 3.01 (s, 3H) |
| A64 | 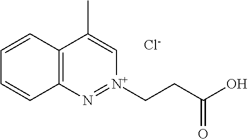 [SMILES: Cc1c[n+](CCC(=O)O)nc2ccccc12 ; [Cl-]] | (DMSO-d₆) 12.76 (bs, 1H), 9.98 (s, 1H), 8.59-8.54 (m, 2H), 8.40-8.33 (m, 2H), 5.25 (t, 2H), 3.28 (t, 2H), 2.99 (s, 3H) |
| A65 | 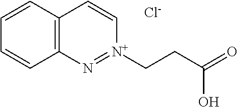 [SMILES: O=C(O)CC[n+]1ccc2ccccc2n1 ; [Cl-]] | (CD₃OD) 9.72 (d, 1H), 9.14 (m, 1H), 8.52 (m, 1H), 8.37 (m, 1H), 8.29-8.21 (m, 2H), 5.34-5.28 (m, 2H), 3.32-3.28 (m, 2H) (CO2H proton missing) |
| A66 | 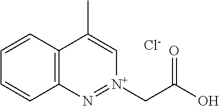 | (300 MHz, CD₃OD) 9.79 (s, 1H), 8.63- 8.58 (m, 2H), 8.45-8.35 (m, 2H), 6.05 (s, 2H), 3.10 (s, 3H) (CO2H proton missing) |
| A67 | 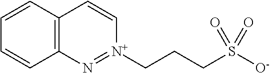 [SMILES: O=S(=O)([O-])CCC[n+]1ccc2ccccc2n1] | (300 MHz, CD₃OD) 9.63 (d, 1H), 9.09 (d, 1H), 8.54 (m, 1H), 8.34 (m, 1H), 8.31-8.19 (m, 2H), 5.22 (t, 2H), 2.85 (t, 2H), 2.59 (pent, 2H) |
| A68 | 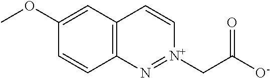 [SMILES: COc1ccc2n[n+](CC(=O)[O-])ccc2c1] | (300 MHz, D₂O) 9.15 (d, 1H), 8.70 (d, 1H) 8.33 (d, 1H), 7.82 (dd, 1H), 7.54 (d, 1H), 5.63 (s, 2H), 4.07 (s, 3H) |
| A69 | 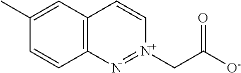 | (300 MHz, CD₃OD) 9.54 (d, 1H), 9.01 (d, 1H), 8.42 (d, 1H), 8.17-8.14 (m, 2H), 5.89 (s, 2H), 2.70 (s, 3H) |
| A70 | 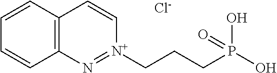 [SMILES: O=P(O)(O)CCC[n+]1ccc2ccccc2n1 ; [Cl-]] | (300 MHz, D₂O) 9.42 (d, 1H), 8.96 (d, 1H), 8.45-8.42 (m, 1H), 8.20-8.11 (m, 3H), 5.04 (t, 2H), 2.42-2.33 (m, 2H), 1.76-1.65 (m, 2H) (POH protons missing) |
| A71 | 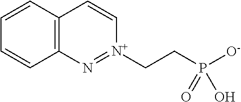 [SMILES: O=P([O-])(O)CC[n+]1ccc2ccccc2n1] | (D₂O) 9.53 (d, 1H), 9.04 (d, 1H), 8.53 (m, 1H), 8.32-8.20 (m, 3H), 5.27-5.21 (m, 2H), 2.59-2.50 (m, 2H) (POH proton missing) |
| A72 | 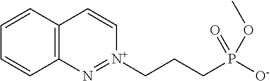 [SMILES: COP(=O)([O-])CCC[n+]1ccc2ccccc2n1] | (D₂O) 9.51 (d, 1H), 9.05 (d, 1H), 8.55 (m, 1H), 8.33-8.20 (m, 3H), 5.12 (t, 2H), 3.50 (d, 3H), 2.42-2.30 (m, 2H), 1.75-1.66 (m, 2H) |
| A73 | 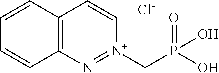 [SMILES: O=P(O)(O)C[n+]1ccc2ccccc2n1 ; [Cl-]] | (D₂O) 9.26-9.33 (m, 2H), 8.43-8.48 (m, 2H), 8.18-8.21 (m, 2H), 5.22 (d, 2H) (POH protons missing) |
| A74 | 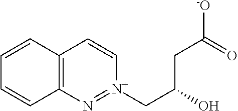 | (CD₃OD) 9.71-9.61 (m, 1H), 9.23 (d, 1H), 8.71-8.61 (m, 1H), 8.51-8.43 (m, 1H), 8.42-8.32 (m, 2H), 5.33 (d, 1H), 5.11 (dd, 1H), 4.75-4.63 (m, 1H), 4.25- 4.12 (m, 2H) (OH proton missing) |
| A75 | 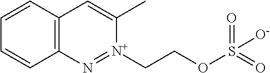 [SMILES: Cc1cc2ccccc2n[n+]1CCOS(=O)(=O)[O-]] | (D₂O) 8.90 (m, 1H), 8.44-8.46 (m, 1H), 8.18-8.20 (m, 3H), 5.35-5.37 (m, 2H), 4.77-4.79 (m, 2H), 3.12 (s, 3H) |
| A76 | 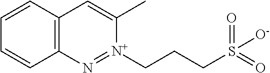 [SMILES: Cc1cc2ccccc2n[n+]1CCCS(=O)(=O)[O-]] | (D₂O) 8.84 (m, 1H), 8.41-8.43 (m, 1H), 8.12-8.14 (m, 3H), 5.11-5.14 (m, 2H), 3.05-3.11 (m, 2H), 2.92 (s, 3H), 2.57- 2.60 (m, 2H) |
| A77 | 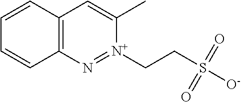 [SMILES: Cc1cc2ccccc2n[n+]1CCS(=O)(=O)[O-]] | (D₂O) 8.93 (s, 1H), 8.48-8.50 (m, 1H), 8.21-8.24 (m, 3H), 5.42 (t, 2H), 3.86 (t, 2H), 3.20 (s, 3H) |
| A78 | 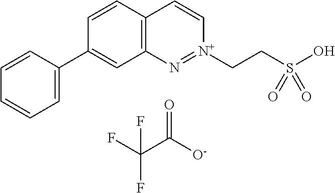 [SMILES: O=S(=O)(O)CC[n+]1ccc2ccc(-c3ccccc3)cc2n1 ; O=C([O-])C(F)(F)F] | (CD₃OD) 9.71 (d, 1H), 9.15 (d, 1H), 8.83 (s, 1H), 8.68 (dd, 1H), 8.48 (d, 1H), 8.00-7.94 (m, 2H), 7.67-7.54 (m, 3H), 5.47 (t, 2H), 3.73 (t, 2H), (SO3H proton missing) |
| A79 | 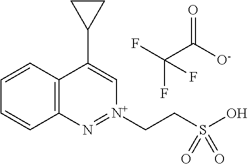 [SMILES: O=S(=O)(O)CC[n+]1cc(C2CC2)c2ccccc2n1 ; O=C([O-])C(F)(F)F] | (CD₃OD) 9.35 (s, 1H), 8.77-8.71 (m, 1H), 8.58-8.51 (m, 1H), 8.34-8.25 (m, 2H), 5.37 (t, 2H), 3.65 (t, 2H), 2.98- 2.89 (m, 1H), 1.62-1.54 (m, 2H), 1.43- 1.35 (m, 2H) (SO3H proton missing) |
| A80 | 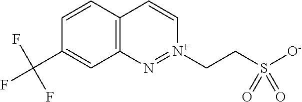 [SMILES: O=S(=O)([O-])CC[n+]1ccc2ccc(C(F)(F)F)cc2n1] | (300 MHz, D₂O) 9.73 (d, 1H), 9.20 (d, 1H), 8.97 (s, 1H), 8.51 (d, 1H), 8.44 (d, 1H), 5.50 (t, 2H), 3.81 (t, 2H) |
| A81 | 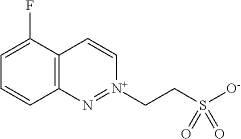 [SMILES: O=S(=O)([O-])CC[n+]1ccc2c(F)cccc2n1] | (300 MHz, D₂O) 9.69-9.61 (m, 1H), 9.26-9.17 (m, 1H), 8.43-8.33 (m, 1H), 8.30-8.18 (m, 1H), 8.01-7.91 (m, 1H), 5.50-5.38 (m, 2H), 3.83-3.71 (m, 2H) |
| A82 | 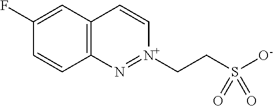 [SMILES: O=S(=O)([O-])CC[n+]1ccc2cc(F)ccc2n1] | (D₂O) 9.53-9.45 (m, 1H), 9.03-8.97 (m, 1H), 8.66-8.59 (m, 1H), 8.13-8.05 (m, 1H), 8.00-7.96 (m, 1H), 5.43-5.36 (m, 2H), 3.80-3.73 (m, 2H) |
| A83 | 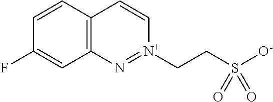 [SMILES: O=S(=O)([O-])CC[n+]1ccc2ccc(F)cc2n1] | (D₂O) 9.58-9.52 (m, 1H), 9.09-9.02 (m, 1H), 8.41-8.32 (m, 1H), 8.19-8.00 (m, 2H), 5.43-5.34 (m, 2H), 3.78-3.70 (m, 2H) |
| A84 | 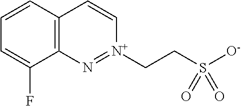 [SMILES: O=S(=O)([O-])CC[n+]1ccc2cccc(F)c2n1] | (300 MHz, D₂O) 9.68-9.59 (m, 1H), 9.18-9.16 (m, 1H), 8.33-8.20 (m, 1H), 8.18-8.10 (m, 1H), 8.04-7.92 (m, 1H), 5.51-5.40 (m, 2H), 3.86-3.73 (m, 2H) |
| A85 | 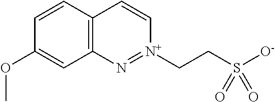 [SMILES: COc1ccc2cc[n+](CCS(=O)(=O)[O-])nc2c1] | (300 MHz, D₂O) 9.42-9.34 (m, 1H), 8.81-8.89 (m, 1H), 8.21-8.12 (m, 1H), 7.88-7.69 (m, 2H), 5.37-5.25 (m, 2H), 5.51-5.40 (m, 2H), 3.86-3.73 (m, 2H) |
| A86 | 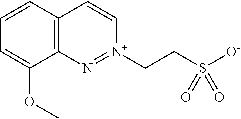 [SMILES: COc1cccc2cc[n+](CCS(=O)(=O)[O-])nc12] | (300 MHz, D₂O) 9.49-9.42 (m, 1H), 8.97-8.86 (m, 1H), 8.24-8.13 (m, 1H), 7.83-7.74 (m, 1H), 7.62-7.54 (m, 1H), 5.43-5.32 (m, 2H), 4.14 (s, 3H), 3.81- 3.70 (m, 2H) |
| A87 | 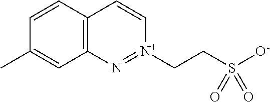 [SMILES: Cc1ccc2cc[n+](CCS(=O)(=O)[O-])nc2c1] | (300 MHz, D₂O) 9.47-9.39 (m, 1H), 8.97-8.86 (m, 1H), 8.28-8.02 (m, 3H), 5.40-5.29 (m, 2H), 3.79-3.68 (m, 2H), 3.62 (s, 3H) |
| A88 | 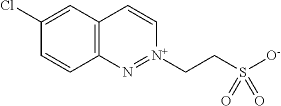 [SMILES: O=S(=O)([O-])CC[n+]1ccc2cc(Cl)ccc2n1] | (300 MHz, D₂O) 9.57-9.50 (m, 1H), 8.99-8.91 (m, 1H), 8.54-8.44 (m, 1H), 8.40-8.32 (m, 1H), 8.25-8.18 (m, 1H), 5.46-5.34 (m, 2H), 3.83-3.72 (m, 2H) |
| A89 | 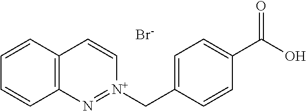 [SMILES: O=C(O)c1ccc(C[n+]2ccc3ccccc3n2)cc1 ; [Br-]] | (CD₃OD) 9.85 (dd, 1H), 9.25 (d, 1H), 8.58-8.66 (m, 1H), 8.42-8.49 (m, 1H), 8.33-8.40 (m, 2H), 8.10 (d, 2H), 7.73 (d, 2H), 6.40 (s, 2H) (CO2H proton missing) |
| A90 | 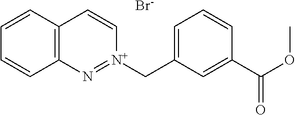 [SMILES: [Br-] ; COC(=O)c1cccc(C[n+]2ccc3ccccc3n2)c1] | (CD₃OD) 9.88 (d, 1H), 9.25 (d, 1H), 8.61 (s, 1H), 8.41-8.48 (m, 1H), 8.36 (d, 2H), 8.30-8.33 (m, 1H), 8.07-8.12 (m, 1H), 7.90-7.96 (m, 1H), 7.55-7.65 (m, 1H), 6.41 (s, 2H), 3.91 (s, 3H) |
| A91 | 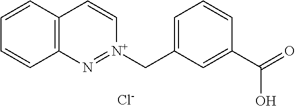 [SMILES: O=C(O)c1cccc(C[n+]2ccc3ccccc3n2)c1 ; [Cl-]] | (CD₃OD) 9.87 (dd, 1H), 9.25 (d, 1H), 8.57-8.69 (m, 1H), 8.41-8.47 (m, 1H), 8.33-8.40 (m, 2H), 8.28-8.32 (m, 1H), 8.11 (d, 1H), 7.90 (d, 1H), 7.60 (t, 1H), 6.40 (s, 2H) (CO2H proton missing) |
| A92 | 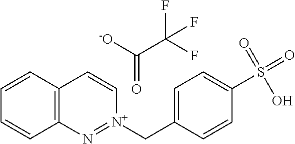 | (CD₃OD) 9.80 (d, 1H), 9.21 (d, 1H), 8.56-8.64 (m, 1H), 8.39-8.45 (m, 1H), 8.29-8.38 (m, 2H), 7.86 (d, 2H), 7.66 (d, 2H), 6.33 (s, 2H) (SO3H proton missing) |
| A93 | 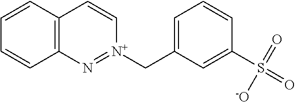 | (CD₃OD) 9.84 (d, 1H), 9.21 (d, 1H), 8.55-8.61 (m, 1H), 8.38-8.44 (m, 1H), 8.28-8.36 (m, 2H), 8.02 (t, 1H), 7.86 (dt, 1H), 7.72 (dt, 1H), 7.50 (t, 1H), 6.35 (s, 2H) |
| A94 | 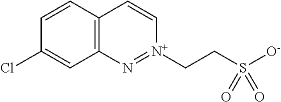 [SMILES: O=S(=O)([O-])CC[n+]1ccc2ccc(Cl)cc2n1] | (D₂O) 9.61-9.58 (d, 1H), 9.09-9.06 (d, 1H), 8.61-8.59 (m, 1H), 8.34-8.30 (m, 1H), 8.23-8.19 (m, 1H), 5.47-5.40 (m, 2H), 3.82-3.76 (m, 2H) |
| A95 | 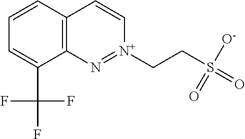 [SMILES: O=S(=O)([O-])CC[n+]1ccc2cccc(C(F)(F)F)c2n1] | (300 MHz, D₂O) 9.78-9.70 (d, 1H), 9.22-9.17 (d, 1H), 8.69-8.60 (m, 1H), 8.54-8.48 (m, 1H), 8.34-8.26 (m, 1H), 5.53-5.44 (m, 2H), 3.84-3.76 (m, 2H) |
| A96 | 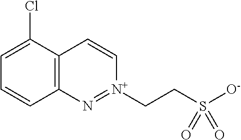 [SMILES: O=S(=O)([O-])CC[n+]1ccc2c(Cl)cccc2n1] | (300 MHz, D₂O) 9.72-9.66 (d, 1H), 9.32-9.26 (d, 1H), 8.52-8.46 (m, 1H), 8.38-8.30 (m, 1H), 8.23-8.14 (m, 1H), 5.49-5.41 (m, 2H), 3.82-3.73 (m, 2H) |
| A97 | 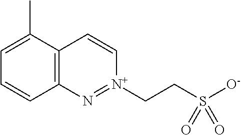 [SMILES: Cc1cccc2n[n+](CCS(=O)(=O)[O-])ccc12] | (D₂O) 9.56-9.50 (d, 1H), 9.10-9.06 (d, 1H), 8.35-8.29 (m, 1H), 8.14-8.08 (m, 1H), 8.05-8.01 (m, 1H), 5.41-5.34 (m, 2H), 3.79-3.72 (m, 2H), 2.74 (s, 3H) |
| A98 | 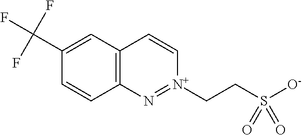 [SMILES: O=S(=O)([O-])CC[n+]1ccc2cc(C(F)(F)F)ccc2n1] | (D₂O) 9.76-9.72 (d, 1H), 9.28-9.24 (d, 1H), 8.76-8.68 (m, 2H), 8.46-8.40 (m, 1H), 5.52-5.46 (m, 2H), 3.82-3.77 (m, 2H) |
| A99 | 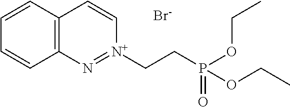 [SMILES: CCOP(=O)(CC[n+]1ccc2ccccc2n1)OCC ; [Br-]] | (CDCl₃) 11.05-10.93 (m, 1H), 9.52 (d, 1H), 8.55-8.45 (m, 2H), 8.27-8.20 (m, 2H), 5.76-5.64 (m, 2H), 4.21-4.05 (m, 4H), 2.92 (td, 2H), 1.25 (s, 6H) |
| A100 | 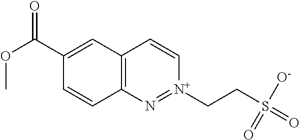 [SMILES: COC(=O)c1ccc2n[n+](CCS(=O)(=O)[O-])ccc2c1] | (D₂O) 9.64-9.62 (d, 1H), 9.18-9.17 (d, 1H), 8.89-8.88 (d, 1H), 8.63- 8.54 (m, 2H), 5.43-5.40 (t, 2H), 4.70 (s, 3H), 3.76-3.73 (t, 2H) |
| A101 | 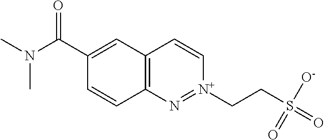 [SMILES: CN(C)C(=O)c1ccc2n[n+](CCS(=O)(=O)[O-])ccc2c1] | (D₂O) 9.58-9.56 (d, 1H), 9.06-9.04 (d, 1H), 8.57-8.54 (d, 1H), 8.29- 8.29 (d, 1H), 8.16-8.14 (t, 1H), 5.40- 5.37 (t, 2H), 3.74-3.71 (t, 2H), 3.05 (s, 3H), 2.89 (s, 3H) |
| A102 | 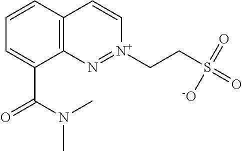 | (D₂O) 9.61 (d, 1H), 9.10 (d, 1H), 8.35 (d, 1H), 8.27 (d, 1H), 8.21 (t, 1H), 5.36 (t, 2H), 3.67 (t, 2H), 3.16 (s, 3H), 2.76 (s, 3H) |
| A103 | 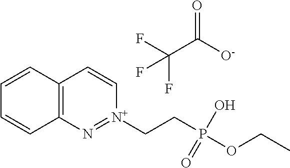 [SMILES: CCOP(=O)(O)CC[n+]1ccc2ccccc2n1 ; O=C([O-])C(F)(F)F] | (CD₃OD) 9.75 (d, 1H), 9.21 (d, 1H), 8.69-8.56 (m, 1H), 8.46-8.33 (m, 3H), 5.33 (td, 2H), 4.02-3.92 (m, 2H), 2.68 (td, 2H), 1.17 (t, 3H) (POH proton missing) |
| A104 | 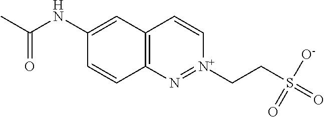 [SMILES: CC(=O)Nc1ccc2n[n+](CCS(=O)(=O)[O-])ccc2c1] | (300 MHz, D₂O) 9.26-9.23 (d, 1H), 8.70-8.68 (d, 1H), 8.55 (s, 1H), 8.36- 8.33 (d, 1H), 8.02-7.99 (d, 1H), 5.27-5.23 (t, 2H), 3.71-3.67 (t, 2H), 2.19 (s, 3H) (NH proton missing) |
| A105 | 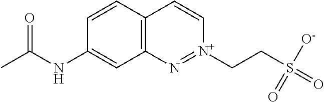 [SMILES: CC(=O)Nc1ccc2cc[n+](CCS(=O)(=O)[O-])nc2c1] | (D₂O) 9.38 (d, 1H), 8.83 (d, 1H), 8.67 (s, 1H), 8.13 (d, 1H), 7.98 (dd, 1H), 5.30 (t, 2H), 3.70 (t, 2H), 2.15 (s, 3H) (NH proton missing) |
| A106 | 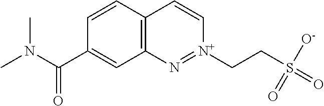 [SMILES: CN(C)C(=O)c1ccc2cc[n+](CCS(=O)(=O)[O-])nc2c1] | (D₂O) 9.60 (d, 1H), 9.08 (d, 1H), 8.55 (s, 1H), 8.37 (d, 1H), 8.17 (d, 1H), 5.41 (t, 2H), 3.75 (t, 2H), 3.09 (s, 3H), 2.96 (s, 3H) |
| A107 | 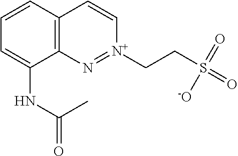 | (D₂O) 9.32 (d, 1H), 8.99 (d, 1H), 8.73 (d, 1H), 8.21 (t, 1H), 8.0 (d, 1H), 5.41 (t, 2H), 3.77 (t, 2H), 2.67 (s, 3H) (NH proton missing) |
| A108 | 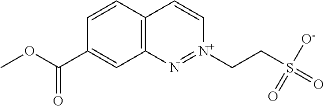 [SMILES: COC(=O)c1ccc2cc[n+](CCS(=O)(=O)[O-])nc2c1] | (D₂O) 9.62 (d, 1H), 9.13 (s, 1H), 9.09 (d, 1H), 8.36 (d, 1H), 8.36 (d, 1H), 5.43 (t, 2H), 3.76 (t, 2H), 4.0 (s, 3H) |
| A109 | 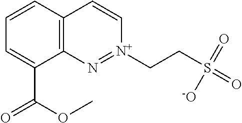 | (D₂O) 9.69 (d, 1H), 9.16 (d, 1H), 8.69 (d, 1H), 8.50 (d, 1H), 8.30 (t, 1H), 5.4 (t, 2H), 4.06 (s, 3H), 3.87 (t, 2H) |
| A110 | 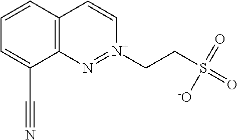 | (D₂O) 9.77 (d, 1H), 9.23 (d, 1H), 8.77 (d, 1H), 8.57 (d, 1H), 8.30 (t, 1H), 5.5 (t, 2H), 3.81 (t, 2H) |
| A111 | 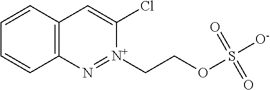 [SMILES: O=S(=O)([O-])OCC[n+]1nc2ccccc2cc1Cl] | (D₂O) 9.15 (s, 1H), 8.50-8.46 (m, 1H), 8.25-8.18 (m, 3H), 5.52-5.47 (m, 2H) (2 protons under water signal) |
| A112 | 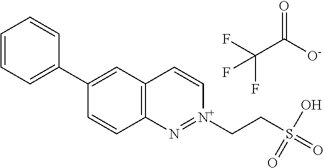 [SMILES: O=S(=O)(O)CC[n+]1ccc2cc(-c3ccccc3)ccc2n1 ; O=C([O-])C(F)(F)F] | (CD₃OD) 9.67 (d, 1H), 9.11 (d, 1H), 8.73-8.63 (m, 3H), 8.02-7.98 (m, 2H), 7.67-7.57 (m, 3H), 5.44 (t, 2H), 3.70 (t, 2H) (SO3H proton missing) |
| A113 | 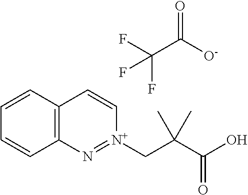 [SMILES: O=C([O-])C(F)(F)F ; CC(C)(C[n+]1ccc2ccccc2n1)C(=O)O] | (CD₃OD) 9.74 (d, 1H), 9.23 (d, 1H), 8.63-8.53 (m, 1H), 8.47-8.34 (m, 3H), 5.31 (s, 2H), 1.39 (s, 6H) (CO2H proton missing) |
| A114 | 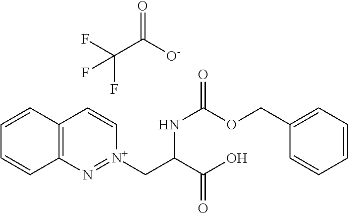 [SMILES: O=C(NC(C[n+]1ccc2ccccc2n1)C(=O)O)OCc1ccccc1 ; O=C([O-])C(F)(F)F] | (CD₃OD) 9.64 (d, 1H), 9.06 (d, 1H), 8.52-8.31 (m, 4H), 7.29 (dd, 3H), 7.24- 7.12 (m, 2H), 5.69-5.57 (m, 1H), 5.41-5.28 (m, 1H), 5.11-5.05 (m, 1H), 4.96-4.84 (m, 2H) (NH and CO2H proton missing) |
| A115 | 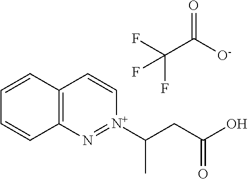 [SMILES: CC(CC(=O)O)[n+]1ccc2ccccc2n1 ; O=C([O-])C(F)(F)F] | (CD₃OD) 9.88-9.72 (m, 1H), 9.21 (dd, 1H), 8.68-8.59 (m, 1H), 8.46-8.32 (m, 3H), 5.85-5.48 (m, 1H), 3.58- 3.18 (m, 2H), 1.84 (d, 3H) (CO2H proton missing) |
| A116 | 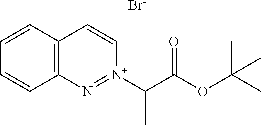 [SMILES: [Br-] ; CC(C(=O)OC(C)(C)C)[n+]1ccc2ccccc2n1] | (CD₃OD) 9.82 (d, 1H), 9.31 (d, 1H), 8.72-8.61 (m, 1H), 8.53-8.45 (m, 1H), 8.45-8.37 (m, 2H), 6.25 (q, 1H), 2.13 (d, 3H), 1.46 (s, 9H) |
| A117 | 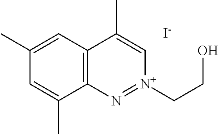 [SMILES: Cc1cc(C)c2n[n+](CCO)cc(C)c2c1 ; [I-]] | (D₂O) 9.18 (s, 1H), 7.90 (s, 1H), 7.83 (s, 1H), 5.06-4.93 (m, 2H), 4.25-4.01 (m, 2H), 2.84-2.70 (m, 6H), 2.56 (s, 3H) (OH proton missing) |
| A118 | 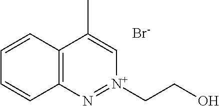 | (DMSO-d₆) 9.88 (s, 1H), 8.63-8.56 (m, 2H), 8.38-8.36 (t, 2H), 5.10- 5.07 (t, 2H), 4.11-4.09 (t, 2H), 3.01 (s, 3H) (OH proton missing) |
| A119 | 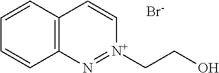 [SMILES: OCC[n+]1ccc2ccccc2n1 ; [Br-]] | (CD₃OD) 9.69 (d, 1H), 9.23 (d, 1H), 8.68-8.61 (m, 1H), 8.49-8.43 (m, 1H), 8.41-8.33 (m, 2H), 5.25-5.18 (m, 2H), 4.28-4.20 (m, 2H) (OH proton missing) |
| A120 | 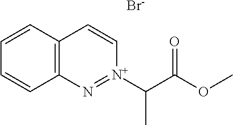 [SMILES: [Br-] ; COC(=O)C(C)[n+]1ccc2ccccc2n1] | (CD₃OD) 9.84 (d, 1H), 9.32 (d, 1H), 8.70-8.61 (m, 1H), 8.54-8.47 (m, 1H), 8.45-8.37 (m, 2H), 6.38 (q, 1H), 3.83 (s, 3H), 2.16 (d, 3H) |
| A121 | 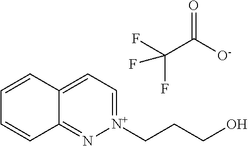 [SMILES: OCCC[n+]1ccc2ccccc2n1 ; O=C([O-])C(F)(F)F] | (CD₃OD) 9.73 (d, 1H), 9.20 (d, 1H), 8.70-8.55 (m, 1H), 8.46-8.41 (m, 1H), 8.38-8.33 (m, 2H), 5.25 (t, 2H), 3.77-3.68 (m, 2H), 2.52-2.39 (m, 2H) (OH proton missing) |
| A122 | 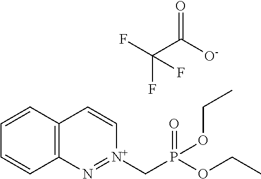 [SMILES: O=C([O-])C(F)(F)F ; CCOP(=O)(C[n+]1ccc2ccccc2n1)OCC] | (CD₃OD) 9.70-9.67 (m, 1H), 9.27 (d, 1H), 8.76-8.29 (m, 4H), 5.88-5.80 (m, 2H), 4.21-4.03 (m, 4H), 1.31-1.19 (m, 6H) |
| A123 | 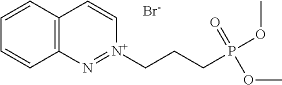 [SMILES: COP(=O)(CCC[n+]1ccc2ccccc2n1)OC ; [Br-]] | (D₂O) 9.62-9.60 (d, 1H), 9.14-9.12 (d, 1H), 8.55-8.53 (m, 1H), 8.36- 8.33 (m, 1H), 8.28-8.25 (m, 2H), 5.12- 5.08 (t, 2H), 3.67-3.64 (d, 6H), 2.48- 2.37 (m, 2H), 2.00-1.91 (m, 2H) |

### Biological Examples

### Post-Emergence Efficacy

Seeds of a variety of test species were sown in standard soil in pots. After cultivation for 14 days (post-emergence) under controlled conditions in a glasshouse (at 24/16° C., day/night; 14 hours light; 65% humidity), the plants were sprayed with an aqueous spray solution derived from the dissolution of the technical active ingredient formula (I) in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted to required concentration using 0.25% or 1% Empicol ESC70 (Sodium lauryl ether sulphate)+1% ammonium sulphate as diluent. The test plants were then grown in a glasshouse under controlled conditions (at 24/16° C., day/night; 14 hours light; 65% humidity) and watered twice daily. After 13 days the test was evaluated (100=total damage to plant; 0=no damage to plant).

The results are shown in Table B (below). A value of n/a indicates that this combination of weed and test compound was not tested/assessed.

### Test Plants:

Ipomoea hederacea (IPOHE), Euphorbia heterophylla (EPHHL), Chenopodium album (CHEAL), Amaranthus palmeri (AMAPA), Lolium perenne (LOLPE), Digitaria sanguinalis (DIGSA), Eleusine indica (ELEIN), Echinochloa crus-galli (ECHCG), Setaria faberi (SETFA)

TABLE B
Control of weed species by compounds of formula (I) after post-emerdence application

| Compound | Application | | | | | | | | | |
|---|---|---|---|---|---|---|---|---|---|---|
| Number | Rate g/Ha | AMAPA | CHEAL | EPHHL | IPOHE | ELEIN | LOLPE | DIGSA | SETFA | ECHCG |
| A1 | 1000 | 100 | 60 | 90 | 50 | 100 | 40 | 70 | 60 | 70 |
| A2 | 1000 | 100 | 80 | 90 | 40 | 100 | 20 | 100 | 80 | 100 |
| A3 | 1000 | 20 | 10 | 20 | 10 | 0 | 20 | 0 | 10 | 0 |
| A4 | 1000 | 40 | 10 | 40 | 30 | 20 | 10 | 10 | 0 | 10 |
| A5 | 1000 | 30 | 20 | 30 | 20 | 10 | 0 | 10 | 10 | 0 |
| A6 | 1000 | 40 | 20 | 30 | 10 | 30 | 10 | 10 | 10 | 10 |
| A7 | 1000 | 30 | 30 | 20 | 10 | 20 | 20 | 0 | 10 | 10 |
| A8 | 1000 | 50 | 60 | 40 | 30 | 50 | 10 | 30 | 10 | 20 |
| A9 | 1000 | 10 | 60 | 50 | 20 | 70 | 10 | 40 | 10 | 10 |
| A10 | 1000 | 30 | 30 | 40 | 40 | 70 | 10 | 50 | 40 | 40 |
| A11 | 1000 | 100 | 100 | 80 | 80 | 80 | 0 | 40 | 70 | 60 |
| A12 | 1000 | 80 | 70 | 50 | 60 | 90 | 40 | 80 | 50 | 40 |
| A13 | 500 | 40 | 50 | 20 | 20 | 30 | 20 | 50 | 60 | 50 |
| A14 | 500 | 100 | 90 | 60 | 50 | 100 | 30 | 80 | 80 | 70 |
| A15 | 1000 | 90 | 80 | 90 | 80 | 80 | 60 | 70 | 60 | 40 |
| A16 | 1000 | 50 | 70 | 40 | 10 | 60 | 20 | 40 | 50 | 50 |
| A17 | 1000 | 70 | 60 | 40 | 30 | 60 | 40 | 40 | 40 | 30 |
| A18 | 1000 | 60 | 100 | 20 | 30 | 90 | 30 | 40 | 20 | 30 |
| A19 | 1000 | 10 | 10 | 20 | 30 | 0 | 0 | 10 | 10 | 10 |
| A20 | 1000 | 30 | 10 | 70 | 0 | 20 | 10 | 20 | 10 | 20 |
| A21 | 1000 | 10 | 50 | 70 | 20 | 0 | 0 | 10 | 0 | 30 |
| A22 | 1000 | 70 | 70 | 70 | 20 | 70 | 20 | 50 | 30 | 40 |
| A23 | 1000 | 60 | 30 | 30 | 20 | 20 | 10 | 40 | 40 | 30 |
| A24 | 1000 | 60 | 60 | 70 | 80 | 80 | 40 | 80 | 60 | 40 |
| A25 | 1000 | 50 | 60 | 30 | 50 | 60 | 50 | 20 | 30 | 0 |
| A26 | 1000 | 100 | 70 | 90 | 70 | 70 | 60 | 70 | 60 | 30 |
| A27 | 1000 | 100 | 90 | 60 | 50 | 80 | 40 | 70 | 70 | 50 |
| A28 | 1000 | 90 | 70 | 80 | 60 | 80 | 60 | 60 | 50 | 40 |
| A30 | 1000 | 70 | 60 | 50 | 10 | 20 | 10 | 20 | 20 | 10 |
| A31 | 1000 | 30 | 20 | 30 | 30 | 0 | 0 | 0 | 0 | 0 |
| A32 | 1000 | 90 | 90 | 90 | 70 | 90 | 30 | 70 | 60 | 80 |
| A33 | 1000 | n/a | 10 | 60 | 30 | 30 | 0 | 40 | 40 | 50 |
| A34 | 1000 | n/a | 20 | 40 | 20 | 30 | 10 | 40 | 20 | 40 |
| A35 | 1000 | n/a | 0 | 30 | 20 | 20 | 10 | 30 | 0 | 20 |
| A36 | 1000 | n/a | n/a | 30 | 30 | 90 | 20 | 80 | 40 | 70 |
| A37 | 1000 | 0 | 0 | 20 | 10 | 60 | 20 | 60 | 40 | 50 |
| A38 | 1000 | 20 | 40 | 30 | 30 | 10 | 10 | 20 | 10 | 30 |
| A39 | 1000 | 10 | 20 | 40 | 30 | 10 | 0 | 10 | 0 | 10 |
| A40 | 500 | 50 | 50 | 40 | 50 | 20 | 0 | 10 | 0 | 30 |
| A41 | 1000 | 100 | 100 | 100 | 80 | 100 | 70 | 80 | 90 | 30 |
| A42 | 1000 | 90 | 50 | 50 | 70 | 80 | 10 | 40 | 50 | 20 |
| A43 | 1000 | 100 | 100 | 100 | 80 | 100 | 40 | 80 | 70 | 70 |
| A44 | 1000 | 30 | 30 | 90 | 20 | 70 | 0 | 30 | 30 | 10 |
| A45 | 1000 | 100 | 80 | 100 | 60 | 100 | 60 | 80 | 80 | 40 |
| A46 | 1000 | 100 | 70 | 100 | 70 | 90 | 40 | 70 | 60 | 30 |
| A47 | 1000 | 100 | 60 | 60 | 20 | 90 | 10 | 90 | 40 | 30 |
| A48 | 1000 | 100 | 80 | 100 | 100 | 90 | 70 | 60 | 70 | 40 |
| A49 | 1000 | 100 | 90 | 90 | 80 | 90 | 50 | 70 | 60 | 60 |
| A50 | 500 | 80 | 70 | 60 | 40 | 20 | 20 | 30 | 20 | 20 |
| A51 | 1000 | 90 | 30 | 60 | 30 | 30 | 10 | 40 | 30 | 20 |
| A52 | 1000 | 100 | 40 | 100 | 30 | 70 | 20 | 40 | 30 | 20 |
| A53 | 500 | 20 | 0 | 20 | 10 | 40 | 0 | 10 | 10 | 0 |
| A54 | 1000 | 30 | 40 | 30 | 10 | 30 | 10 | 30 | 20 | 10 |
| A55 | 1000 | 10 | 30 | 30 | 30 | 10 | 0 | 10 | 0 | 20 |
| A56 | 1000 | 0 | 0 | 50 | 0 | 30 | 0 | 20 | 20 | 10 |
| A57 | 1000 | 70 | 40 | 60 | 20 | 60 | 10 | 40 | 30 | 20 |
| A58 | 1000 | 0 | 30 | 20 | 10 | 10 | 0 | 0 | 0 | 10 |
| A59 | 1000 | 30 | 60 | 20 | 10 | 10 | 10 | 20 | 20 | 20 |
| A60 | 1000 | 0 | 0 | 10 | 30 | 40 | 10 | 30 | 10 | 20 |
| A61 | 1000 | 70 | 60 | 70 | 60 | 90 | 10 | 80 | 50 | 60 |
| A62 | 1000 | 70 | 60 | 40 | 50 | 70 | 40 | 50 | 30 | 30 |
| A63 | 1000 | 40 | 50 | 20 | 10 | 40 | 30 | 20 | 10 | 10 |
| A64 | 1000 | 70 | 70 | 70 | 20 | 50 | 10 | 50 | 30 | 40 |
| A65 | 1000 | 80 | 80 | 90 | 60 | 90 | 40 | 90 | 60 | 70 |
| A66 | 1000 | 20 | 60 | 20 | 30 | 20 | 0 | 0 | 0 | 0 |
| A67 | 1000 | 100 | 90 | 70 | 80 | 80 | 80 | 80 | 80 | 60 |
| A68 | 1000 | 60 | 50 | 20 | 20 | 20 | 10 | 30 | 30 | 50 |
| A69 | 1000 | 30 | 0 | 40 | 0 | 30 | 0 | 30 | 30 | 40 |
| A70 | 1000 | 80 | 90 | 80 | 100 | 90 | 40 | 80 | 60 | 40 |
| A71 | 1000 | 70 | 100 | 90 | 40 | 70 | 30 | 80 | 70 | 40 |
| A72 | 1000 | 70 | 70 | 50 | 100 | 70 | 50 | 50 | 60 | 50 |
| A73 | 1000 | 10 | 0 | 10 | 30 | 10 | 10 | 10 | 0 | 10 |
| A74 | 1000 | 0 | 0 | 10 | 10 | 0 | 0 | 0 | 0 | 0 |
| A75 | 1000 | 100 | 90 | 40 | 30 | 80 | 20 | 70 | 30 | 30 |
| A76 | 1000 | 60 | 60 | 50 | 40 | 60 | 30 | 60 | 60 | 60 |
| A77 | 1000 | 100 | 100 | 90 | 80 | 90 | 40 | 90 | 30 | 30 |
| A78 | 1000 | 20 | 0 | 40 | 10 | 70 | 0 | 50 | 30 | 20 |
| A79 | 1000 | 60 | 30 | 40 | 40 | 10 | 10 | 20 | 30 | 20 |
| A80 | 1000 | 0 | 20 | 20 | 0 | 30 | 10 | 20 | 30 | 20 |
| A81 | 1000 | 40 | 70 | 90 | 10 | 30 | 20 | 20 | 20 | 20 |
| A82 | 1000 | 90 | 70 | 60 | 40 | 60 | 30 | 40 | 30 | 20 |
| A83 | 1000 | 100 | 100 | 100 | 60 | 80 | 50 | 50 | 50 | 40 |
| A84 | 1000 | 100 | 70 | 100 | 50 | 70 | 50 | 60 | 50 | 20 |
| A85 | 1000 | 100 | 100 | 80 | 70 | 40 | 20 | 20 | 30 | 20 |
| A86 | 1000 | 100 | 100 | 90 | 90 | 80 | 60 | 40 | 50 | 50 |
| A87 | 1000 | 100 | 100 | 100 | 70 | 80 | 40 | 40 | 50 | 40 |
| A88 | 1000 | 70 | 40 | 60 | 20 | 50 | 30 | 50 | 40 | 20 |
| A89 | 1000 | 20 | 0 | 40 | 20 | 0 | 0 | 0 | 0 | 0 |
| A90 | 1000 | 20 | n/a | 30 | 10 | 20 | 0 | 10 | 0 | 10 |
| A91 | 1000 | n/a | 20 | 40 | 10 | 20 | 10 | 20 | 20 | 40 |
| A92 | 1000 | n/a | 0 | 50 | 40 | 30 | 10 | 20 | 10 | 30 |
| A93 | 1000 | 10 | 10 | 50 | 40 | 10 | 10 | 20 | 20 | 20 |
| A94 | 1000 | 100 | 90 | 100 | 40 | 90 | 40 | 50 | 50 | 40 |
| A95 | 1000 | 40 | 50 | 50 | 10 | 10 | 0 | 10 | 10 | 10 |
| A96 | 1000 | 70 | 100 | 100 | 20 | 60 | 40 | 60 | 30 | 40 |
| A97 | 500 | 100 | 90 | 80 | 10 | 70 | 10 | 50 | 60 | 30 |
| A98 | 500 | 0 | 30 | 40 | 0 | 20 | 10 | 10 | 0 | 10 |
| A99 | 500 | 10 | 20 | 40 | 10 | 30 | 20 | 50 | 50 | 40 |
| A100 | 500 | 30 | 30 | 40 | 30 | 10 | 0 | 10 | 20 | 10 |
| A101 | 500 | n/a | 60 | 70 | 40 | 80 | 20 | 70 | 70 | 60 |
| A102 | 500 | n/a | 100 | 60 | 70 | 100 | 30 | 80 | 90 | 70 |
| A103 | 500 | 20 | 0 | 40 | 20 | 30 | 20 | 60 | 60 | 30 |
| A104 | 125 | n/a | 30 | 50 | 10 | 80 | 20 | 60 | 70 | 60 |
| A105 | 500 | n/a | 70 | 30 | 20 | 80 | 20 | 60 | 70 | 50 |
| A106 | 500 | n/a | 80 | 30 | 20 | 100 | 30 | 70 | 100 | 80 |
| A107 | 130 | 60 | 40 | 50 | 40 | 40 | 40 | 70 | 70 | 80 |
| A108 | 500 | 20 | 40 | 50 | 10 | 30 | 10 | 70 | 10 | 20 |
| A109 | 500 | 40 | 20 | 30 | 10 | 10 | 10 | 10 | 10 | 30 |
| A110 | 500 | 70 | 30 | 60 | 40 | 20 | 0 | 20 | 20 | 20 |
| A111 | 1000 | 0 | 0 | 30 | 0 | 20 | 0 | 10 | 0 | 0 |
| A112 | 1000 | 10 | 30 | 20 | 10 | 30 | 10 | 30 | 20 | 10 |
| A113 | 500 | 10 | 10 | 10 | 10 | 30 | 20 | 30 | 20 | 40 |
| A114 | 500 | 50 | 40 | 50 | 30 | 30 | 10 | 20 | 20 | 20 |
| A115 | 500 | 100 | 40 | 20 | 20 | 60 | 20 | 60 | 30 | 20 |
| A116 | 500 | 60 | 50 | 30 | 10 | 30 | 20 | 40 | 30 | 30 |
| A117 | 1000 | 30 | 20 | 20 | 20 | 60 | 10 | 20 | 30 | 20 |
| A118 | 1000 | 10 | 10 | 20 | 10 | 20 | 10 | 20 | 10 | 10 |
| A119 | 1000 | 90 | 90 | 80 | 50 | 40 | 20 | 50 | 50 | 20 |
| A120 | 500 | 100 | 90 | 60 | 20 | 60 | 0 | 50 | 50 | 30 |
| A121 | 1000 | 30 | 60 | 30 | 20 | 0 | 10 | 0 | 30 | 10 |

1. Use of a compound of formula (I) or an agronomically acceptable salt or zwitterionic species thereof, as a herbicide: 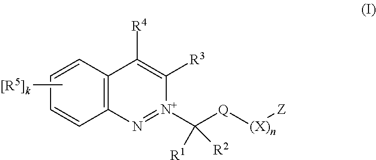
wherein:
R¹ is selected from the group consisting of hydrogen, halogen, C₁-C₆alkyl, C₂-C₆alkenyl, C₁-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O;
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³ is selected from the group consisting of hydrogen, halogen, C₁-C₆alkyl, C₁-C₆haloalkyl and C₁-C₆alkoxy;
R⁴ is selected from the group consisting of hydrogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, which may be the same or different, and wherein the aforementioned CR¹R², Q and Z moieties may be attached at any position of said cycloalkyl, phenyl, heteroaryl or heterocyclyl moieties;
n is 0 or 1;
k is 0, 1, 2, 3 or 4;
when k is 1 or 2, each R⁵ is independently selected from the group consisting of halogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
or,
when k is 3 or 4, each R⁵ is independently selected from the group consisting of halogen, C₁-C₆alkyl, C₁-C₆haloalkyl, C₁-C₆alkoxy and C₁-C₆haloalkoxy;
each R⁶ is independently selected from hydrogen and C₁-C₆alkyl;
R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
each R⁸ is independently selected from the group consisting of hydrogen and C₁-C₄alkyl;
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl, phenyl and benzyl, and wherein said phenyl or benzyl are optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2.

2. A compound of formula (I) or an agronomically acceptable salt or zwitterionic species thereof, 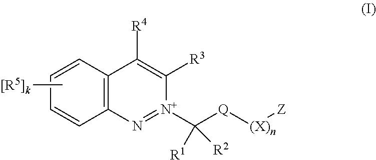
wherein:
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, μ-OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O;
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³ is selected from the group consisting of hydrogen, halogen, C₁-C₆alkyl, C₁-C₆haloalkyl and C₁-C₆alkoxy;
R⁴ is selected from the group consisting of hydrogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, which may be the same or different, and wherein the aforementioned CR¹R², Q and Z moieties may be attached at any position of said cycloalkyl, phenyl, heteroaryl or heterocyclyl moieties;
n is 0 or 1;
k is 0, 1, 2, 3 or 4;
when k is 1 or 2, each R⁵ is independently selected from the group consisting of halogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
or,
when k is 3 or 4, each R⁵ is independently selected from the group consisting of halogen, C₁-C₆alkyl, C₁-C₆haloalkyl, C₁-C₆alkoxy and C₁-C₆haloalkoxy;
each R⁶ is independently selected from hydrogen and C₁-C₆alkyl;
R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
each R⁸ is independently selected from the group consisting of hydrogen and C₁-C₄alkyl;
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl, phenyl and benzyl, and wherein said phenyl or benzyl are optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2
with the proviso that the compound of formula (I) is not:
i) selected from the group consisting of 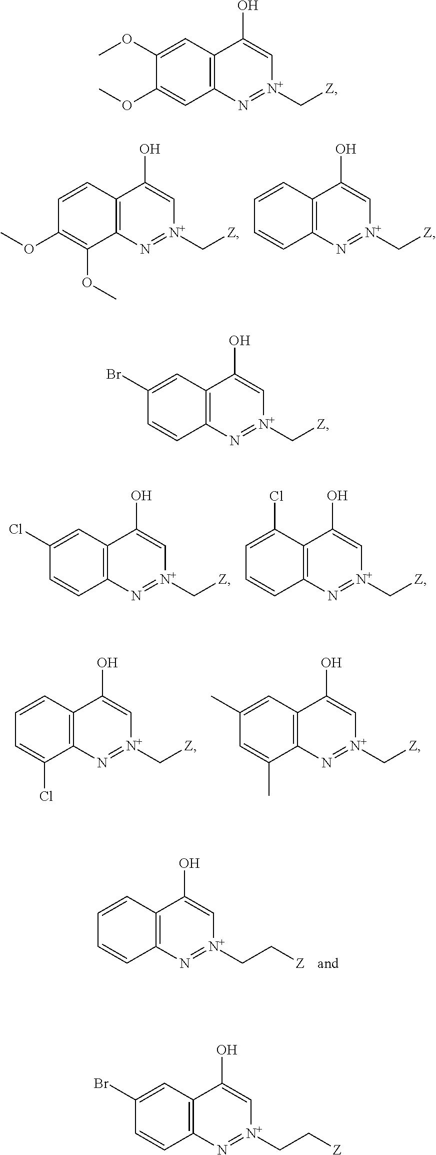
wherein Z is —CH₂OH, —C(O)OH or —C(O)OCH₂CH₃;
or
ii) the compound:

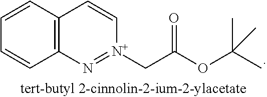 [SMILES: CC(C)(C)OC(=O)C[n+]1ccc2ccccc2n1]

3. The compound of formula (I) according to claim 2, wherein R¹ and R² are independently selected from the group consisting of hydrogen and C₁-C₃alkyl.

4. The compound of formula (I) according to claim 2, wherein each R^{1a} and R^{2b} are independently selected from the group consisting of hydrogen, C₁-C₆alkyl, —OH and —NH₂.

5. The compound of formula (I) according to claim 2, wherein m is 1 or 2.

6. The compound of formula (I) according to claim 2, wherein R³ is selected from the group consisting of hydrogen, halogen and C₁-C₃alkyl.

7. The compound of formula (I) according to claim 2, wherein R⁴ is selected from the group consisting of hydrogen, —NH₂, —NR⁶R⁷, —OR⁷, —S(O)ᵣR¹², C₁-C₃alkyl, C₁-C₃haloalkyl, C₃-C₆cycloalkyl, C₂-C₄alkenyl, C₂-C₄alkynyl, C₁-C₃haloalkoxy, C₁-C₃alkylaminocarbonyl and phenyl, wherein said phenyl is optionally substituted by 1, 2 or 3 R⁹ substituents, which may be the same or different.

8. The compound of formula (I) according to claim 2, wherein when k is 1 or 2, each R⁵ is independently selected from the group consisting of halogen, cyano, —NH₂, —NR⁶R⁷, —OH, —OR⁷, C₁-C₃alkyl, C₁-C₃haloalkyl, C₃-C₆cycloalkyl, C₁-C₃haloalkoxy, C₂-C₄alkenyl, C₂-C₄alkynyl, C₁-C₃alkoxycarbonyl, C₁-C₃alkylaminocarbonyl, di-C₁-C₃alkylaminocarbonyl and phenyl, wherein said phenyl is optionally substituted by 1, 2 or 3 R⁹ substituents, which may be the same or different.

9. The compound of formula (I) according to claim 2, wherein k is 0 or 1.

10. The compound of formula (I) according to claim 2, wherein Z is selected from the group consisting of —C(O)OR¹⁰, —CH₂OH, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰, —P(O)(R¹³)(OR¹⁰) and tetrazole.

11. The compound of formula (I) according to claim 2, wherein Z is selected from the group consisting of —C(O)OH, —C(O)NHS(O)₂CH₃, —S(O)₂OH, —OS(O)₂OH and —NHS(O)₂OH.

12. The compound of formula (I) according to claim 2, wherein n is 0.

13. The use of a compound of formula (I) as defined in claim 2, or an agronomically acceptable salt or zwitterionic species thereof, as a herbicide.

14. An agrochemical composition comprising a herbicidally effective amount of a compound of formula (I) as defined in any claim 2 and an agrochemically-acceptable diluent or carrier.

15. The composition according to claim 14, further comprising at least one additional active ingredient.

16. A method of controlling unwanted plant growth, comprising applying a compound of formula (I) or an agronomically acceptable salt or zwitterionic species thereof: 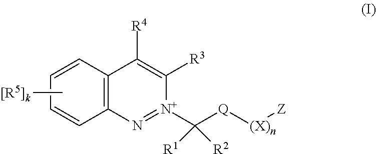
wherein:
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O;
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³ is selected from the group consisting of hydrogen, halogen, C₁-C₆alkyl, C₁-C₆haloalkyl and C₁-C₆alkoxy;
R⁴ is selected from the group consisting of hydrogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, which may be the same or different, and wherein the aforementioned CR¹R², Q and Z moieties may be attached at any position of said cycloalkyl, phenyl, heteroaryl or heterocyclyl moieties;
n is 0 or 1;
k is 0, 1, 2, 3 or 4;
when k is 1 or 2, each R⁵ is independently selected from the group consisting of halogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different;
or,
when k is 3 or 4, each R⁵ is independently selected from the group consisting of halogen, C₁-C₆alkyl, C₁-C₆haloalkyl, C₁-C₆alkoxy and C₁-C₆haloalkoxy;
each R⁶ is independently selected from hydrogen and C₁-C₆alkyl;
R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
each R⁸ is independently selected from the group consisting of hydrogen and C₁-C₄alkyl;
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl, phenyl and benzyl, and wherein said phenyl or benzyl are optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2,
to the unwanted plants or to the locus thereof.
